SEQUENCE LISTING (2 sequences)

SEQ ID NO: 1 - DNA, 16 bp, Artificial Sequence
  Synthetic Oligonucleotide
aataatctcatgtcag

SEQ ID NO: 2 - DNA, 3731 bp, Homo sapiens
gtccgatggggctctggtggcgtgatctgcgcgccccaggcgtcaagcacccacaccctagaaggtttccgcagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaacaaaaaaaaaaaaaaaaaaaaa

(19) United States
(12) Patent Application Publication
(10) Pub. No.: US 2023/0355653 A1
(43) Pub. Date: Nov. 9, 2023

(54) PCSK9 INHIBITORS AND METHODS OF TREATMENT USING SAME

(71) Applicant: AstraZeneca AB, Södertälje (SE)

(72) Inventors: Tina RYDÉN-BERGSTEN, Gaithersburg, MD (US); Dinko REKIC, Södertälje (SE); Catarina NILSSON, Södertälje (SE); Jane KNÖCHEL, Södertälje (SE)

(21) Appl. No.: 18/245,603

(22) PCT Filed: Sep. 16, 2021
(86) PCT No.: PCT/EP2021/075546

§ 371 Date: Mar. 16, 2023

Related U.S. Application Data
(60) Provisional application No. 63/079,947, filed on Sep. 17, 2020.
(60) Provisional application No. 63/104,107, filed on Oct. 22, 2020.
(60) Provisional application No. 63/122,199, filed on Dec. 7, 2020.
(60) Provisional application No. 63/140,373, filed on Jan. 22, 2021.

(51) Int. Cl. A61K 31/7125; A61P 3/06; A61K 9/00

(52) U.S. Cl. CPC A61K 31/7125; A61P 3/06; A61K 9/0019

(57) ABSTRACT

The present disclosure provides dosages and methods for treating a disease associated with proprotein convertase subtilisin/kexin type 9 (PCSK9). The present disclosure also provides unit dosages, dosing regimens and methods for treating a disease associated with PCSK9.

### SUMMARY

The present disclosure provides dosages and methods for treating a disease associated with proprotein convertase subtilisin/kexin type 9 (PCSK9).

PCSK9 is an enzyme with an important role in lipoprotein metabolism. PCSK9 increases the levels of circulating LDL cholesterol (LDL-C) by increasing degradation of the LDL receptors. Rare gain-of-function mutations in PCSK9 lead to a high LDL-C level and premature coronary heart disease, whereas loss-of-function variants lead to a low LDL-C level and a reduced incidence of coronary heart disease (Zhao et al., Am. J. Hum. Genet. 2006, 79: 514-523; Horton et al., J. Lipid Res. 2009, 50: Suppl: S172-S177). Therefore, PCSK9 is a well-validated target for LDL-cholesterol-lowering therapy (Hooper et al., Expert Opin. Biol. Ther. 2013, 13: 429-435).

Antibodies for blocking PCSK9, Alirocumab and Evolocumab, have been demonstrated to reduce circulating PCSK9 levels and lower LDL-cholesterol levels but have a short duration of action, necessitating frequent subcutaneous injections (Zhang et al., BMC Med. 2015, 13: 123; Navarese et al., Ann. Intern. Med. 2015, 163: 40-51).

Because cardiovascular disease often requires long-term management, ease of dosing and administration are important for patient compliance. There remains a need for PCSK9 inhibitors with increased efficacy and greater ease of administration, for the treatment of diseases such as cardiovascular diseases, dyslipidemias, mixed dyslipidemias, and hypercholesterolemia.

The present disclosure provides dosages and methods for treating a disease associated with PCSK9.

In one aspect, a unit dosage is provided that includes from about 10 mg to about 120 mg of a PCSK9 antisense oligonucleotide of Formula I (SEQ ID NO: 1), as shown in FIG. 1-3, or a pharmaceutically acceptable salt thereof.

In one aspect, a unit dosage is provided that includes from about 10 mg to about 120 mg of a PCSK9 antisense oligonucleotide or pharmaceutically acceptable salt thereof, wherein the PCSK9 antisense oligonucleotide includes a modified oligonucleotide and a conjugate group. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

In one aspect, the unit dosage includes at least about 10 mg, about 20 mg, about 30 mg, about 40 mg or about 50 mg and up to about 60 mg, about 70 mg, about 80 mg, about 90 mg, about 100 mg, about 110 mg or about 120 mg of the PCSK9 antisense oligonucleotide or a pharmaceutically acceptable salt thereof. In one aspect, the unit dosage includes about 10 mg, about 20 mg, about 30 mg, about 40 mg, about 50 mg, about 60 mg, about 70 mg, about 80 mg, about 90 mg, about 100 mg, about 110 mg or about 120 mg of the PCSK9 antisense oligonucleotide or a pharmaceutically acceptable salt thereof. In one aspect, the unit dosage includes from about 50 mg to about 100 mg of the PCSK9 antisense oligonucleotide or a pharmaceutically acceptable salt thereof. In one aspect, the unit dosage includes about 15 mg, about 50 mg, about 70 mg or about 90 mg of the PCSK9 antisense oligonucleotide or a pharmaceutically acceptable salt thereof.

In one aspect, the unit dosage includes about 50 mg of the PCSK9 antisense oligonucleotide or a pharmaceutically acceptable salt thereof

In one aspect, the unit dosage is formulated for parenteral administration. In one aspect, parenteral administration includes subcutaneous, intramuscular or intravenous administration. In one aspect, the unit dosage is formulated for subcutaneous administration.

In one aspect, the pharmaceutically acceptable salt is a sodium salt. In one aspect, the pharmaceutically acceptable salt is a potassium salt.

In one aspect, the unit dosage includes a pharmaceutically acceptable carrier, adjuvant or excipient.

In one aspect, the unit dosage is contained in a pre-filled syringe. In one aspect, the unit dosage is contained in a single-dose pre-filled syringe.

In one aspect, a method of reducing a low-density lipoprotein cholesterol (LDL-C) level in a subject is provided. In one aspect, the method includes administering to the subject a unit dosage that includes from about 10 mg to about 120 mg of a PCSK9 antisense oligonucleotide of Formula I, or a pharmaceutically acceptable salt thereof.

In one aspect, a method of reducing a level of PCSK9 in a subject is provided. In one aspect, the method includes administering to the subject a unit dosage that includes from about 10 mg to about 120 mg of a PCSK9 antisense oligonucleotide of Formula I, or a pharmaceutically acceptable salt thereof.

In one aspect, a method of reducing a risk of cardiovascular disease in a subject is provided.

In one aspect, the method includes administering to the subject a unit dosage that includes from about 10 mg to about 120 mg of a PCSK9 antisense oligonucleotide of Formula I, or a pharmaceutically acceptable salt thereof.

In one aspect, a unit dosage that includes from about 10 mg to about 120 mg of a PCSK9 antisense oligonucleotide of Formula I, or a pharmaceutically acceptable salt thereof is used in the manufacture of a medicament for the reduction of a low-density lipoprotein cholesterol (LDL-C) level in a subject. In one aspect, the unit dosage is used in the manufacture of a medicament for the treatment of disease associated with PCSK9. In one aspect, the disease associated with PCSK9 is selected from: elevated low-density lipoprotein cholesterol (LDL-C) levels, hyperlipidemia, dyslipidemia, atherosclerosis, cardiovascular disease, hypercholesterolemia, and combinations thereof. In one aspect, the disease associated with PCSK9 is dyslipidemia or hypercholesterolemia.

In one aspect, a method of reducing a low-density lipoprotein cholesterol (LDL-C) level in a subject is provided. In one aspect, the method includes administering to the subject a unit dosage that includes from about 10 to about 120 mg of a PCSK9 antisense oligonucleotide of Formula I (SEQ ID NO:1), as shown in FIG. 1-3, or pharmaceutically acceptable salt thereof. In one aspect, the method includes administering to the subject a unit dosage that includes from about 10 mg to about 120 mg of a PCSK9 antisense oligonucleotide or pharmaceutically acceptable salt thereof, wherein the PCSK9 antisense oligonucleotide includes a modified oligonucleotide and a conjugate group. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

In one aspect, a method of reducing a low-density lipoprotein cholesterol (LDL-C) level in a subject is provided. In one aspect, the method includes administering to the subject a unit dosage that includes from about 10 mg to about 120 mg of a PCSK9 antisense oligonucleotide or pharmaceutically acceptable salt thereof. In one aspect, the PCSK9 antisense oligonucleotide includes a modified oligonucleotide and a conjugate group. In one aspect, the modified oligonucleotide consists of SEQ ID NO:1 and includes a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

In one aspect, the level of LDL-C in the subject is reduced about 10% to about 90% after administration of the unit dosage as compared to a level of LDL-C in the subject before administration of the unit dosage. In one aspect, the level of LDL-C in the subject is reduced at least about 10%, at least about 20%, at least about 30%, at least about 40%, at least about 50%, at least about 60%, at least about 70%, and up to about 80%, or about 90% after administration of the unit dosage as compared to a level of LDL-C in the subject before administration of the unit dosage. In one aspect, the level of LDL-C in the subject is reduced about 20% to about 80%, about 30% to about 70%, about 40% to about 70%, or about 50% to about 70% after administration of the unit dosage as compared to a level of LDL-C in the subject before administration of the unit dosage.

In one aspect, the level of LDL-C in the subject is reduced for at least about 2 weeks after administering the unit dosage as compared to a level of LDL-C in the subject before administration of the unit dosage. In one aspect, the level of LDL-C in the subject is reduced for at least about 3 weeks, about 4 weeks, or about 5 weeks and up to about 6 weeks after administering the unit dosage as compared to a level of LDL-C in the subject before administration of the unit dosage.

In one aspect, a plasma level of LDL-C of the subject is reduced. In one aspect, a serum level of LDL-C of the subject is reduced.

In one aspect, the subject has a serum low-density lipoprotein cholesterol (LDL-C) level of at least about 100 mg/dL, at least about 110 mg/dL, at least about 120 mg/dL, at least about 130 mg/dL, at least about 140 mg/dL, at least about 150 mg/dL, at least about 160 mg/dL, at least about 170 mg/dL, at least about 180 mg/dL, at least about 190 mg/dL, at least about 200 mg/dL, at least about 300 mg/dL or at least about 400 mg/dL prior to administration of the unit dosage.

In one aspect, a method of treating a disease associated with PCSK9 in a subject is provided. In one aspect, the method includes administering to the subject from about 10 to about 120 mg of a PCSK9 antisense oligonucleotide of Formula I (SEQ ID NO:1), as shown in FIG. 1-3, or pharmaceutically acceptable salt thereof. In one aspect, the method includes administering to the subject a unit dosage that includes from about 10 mg to about 120 mg of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide. In one aspect, the disease associated with PCSK9 is selected from: elevated low-density lipoprotein cholesterol (LDL-C) levels, hyperlipidemia, dyslipidemia, atherosclerosis, cardiovascular disease, hypercholesterolemia, and combinations thereof. In one aspect, the disease associated with PCSK9 is dyslipidemia or hypercholesterolemia.

In one aspect, a level of PCSK9 in the subject is reduced. In one aspect, the level of PCSK9 in the subject is reduced about 10% to about 95% after administration of the unit dosage as compared to a level of PCSK9 in the subject before administration of the unit dosage. In one aspect, the level of PCSK9 in the subject is reduced at least about 10%, at least about 20%, at least about 30%, at least about 40%, at least about 50% at least about 60%, or at least about 70% and up to about 80%, or about 90% after administration of the unit dosage as compared to a level of LDL-C in the subject before administration of the unit dosage. In one aspect, the level of PCSK9 in the subject is reduced about 10% to about 95%, about 20% to about 80%, about 30% to about 70%, about 40% to about 70%, or about 50% to about 70% after administration of the unit dosage as compared to a level of PCSK9 in the subject before administration of the unit dosage.

In one aspect, the level of PCSK9 in the subject is reduced for at least about 2 weeks after administering the PCSK9 antisense oligonucleotide as compared to a level of PCSK9 in the subject before administration of the PCSK9 antisense oligonucleotide. In one aspect, the level of PCSK9 in the subject is reduced for at least about 3 weeks, at least about 4 weeks, at least about 5 weeks and up to about 6 weeks after administration of the PCSK9 antisense oligonucleotide as compared to a level of PCSK9 in the subject before administration of the PCSK9 antisense oligonucleotide. In one aspect, a plasma level of PCSK9 in the subject is reduced. In one aspect, a serum level of PCSK9 in the subject is reduced. In one aspect, PCSK9 expression is reduced.

In one aspect, the unit dosage is administered to the subject about every 2 weeks to about every 6 weeks. In one aspect, the unit dosage is administered to the subject about once every 2 weeks, about every 3 weeks, about every 4 weeks, about every 5 weeks or about every 6 weeks, for at least about 6 weeks, at least about 12 weeks, at least about 26 weeks, or at least about 52 weeks. In one aspect, the unit dosage is administered to the subject once a month for at least about 3 months. In one aspect, the unit dosage is administered to the subject once a month for at least about at least about 6 months, at least about 9 months, at least about 12 months, at least about 15 months or at least about 24 months. In one aspect, the unit dosage is administered to the subject once every other month for at least about 4 months. In one aspect, the unit dosage is administered to the subject once every other month for at least about at least about 6 months, at least about 12 months, at least about 18 months, at least about 24 months or at least about 30 months or at least about 36 months.

In one aspect, the unit dosage is administered to the subject about every 21 days, about every 28 days, or about every 30 days for at least about 90 days, at least about 120 days, at least about 180 days, or at least about 365 days.

In one aspect, the unit dosage is administered by parenteral administration. In one aspect, parenteral administration includes subcutaneous, intravenous, or intramuscular administration. In one aspect, parenteral administration includes subcutaneous administration.

In one aspect, the subject is a human.

In one aspect, a dosing regimen is provided for reducing a low-density lipoprotein cholesterol (LDL-C) level in a subject. In one aspect, the method includes administering to the subject a loading dose that includes a unit dosage that includes from about 10 mg, to about 120 mg of a PCSK9 antisense oligonucleotide of Formula I (SEQ ID NO: 1), as shown in FIG. 1-3, or a pharmaceutically acceptable salt thereof. In one aspect, the LDL-C level in the subject is reduced about 10% to about 90% after administering the loading dose to provide a reduced LDL-C level in the subject. In one aspect, the dosing regimen includes administering one or more maintenance doses that include a unit dosage that includes from about 10 mg, to about 120 mg of a PCSK9 antisense oligonucleotide of Formula I (SEQ ID NO: 1), as shown in FIG. 1-3, or a pharmaceutically acceptable salt thereof.

In one aspect, the method includes administering to the subject a loading dose that includes a unit dosage that includes from about 10 mg to about 120 mg of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide. In one aspect, the LDL-C level in the subject is reduced about 10% to about 90% after administering the loading dose to provide a reduced LDL-C level in the subject. In one aspect, the dosing regimen includes administering one or more maintenance doses includes a unit dosage that includes from about 10 mg to about 120 mg of a PCSK9 antisense oligonucleotide or pharmaceutically acceptable salt thereof, wherein the PCSK9 antisense oligonucleotide includes a modified oligonucleotide and a conjugate group, wherein the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

In one aspect, the loading dose includes a greater amount of a PCSK9 antisense oligonucleotide of Formula I (SEQ ID NO: 1) or a pharmaceutically acceptable salt thereof than the maintenance dose. In one aspect, the loading dose includes a same amount of a PCSK9 antisense oligonucleotide of Formula I (SEQ ID NO: 1) or a pharmaceutically acceptable salt thereof as the maintenance dose.

In one aspect, the loading dose includes a greater amount than a maintenance dose of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment than the maintenance dose. In one aspect, the loading dose includes a same amount as a maintenance dose of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides.

In one aspect, the maintenance dose is administered to the subject about every 2 weeks to about every 6 weeks. In one aspect, the maintenance dose is administered to the subject about every 2 weeks, about every 3 weeks, about every 4 weeks, about every 5 weeks or about every 6 weeks, for at least about 6 weeks, at least about 12 weeks, at least about 26 weeks, or at least about 52 weeks. In one aspect, the maintenance dose is administered to the subject at least once a month for at least about 3 months. In one aspect, the maintenance dose is administered to the subject about once a month for at least about 6 months, at least about 9 months, at least about 12 months, at least about 15 months, or at least about 24 months. In one aspect, the maintenance dose is administered to the subject about every 21 days, about every 28 days, or about every 30 days for at least about 90 days, at least about 120 days, at least about 180 days, or at least about 365 days.

In one aspect, the loading dose, maintenance dose, or both are administered by parenteral administration. In one aspect, parenteral administration includes subcutaneous, intravenous, or intramuscular administration. In one aspect, parenteral administration includes subcutaneous administration.

In one aspect, the level of LDL-C in the subject is reduced at least about 20%, about 30%, about 40%, about 50%, about 60%, or about 70% and up to about 80%, or about 90% after administering the loading dose. In one aspect, the level of LDL-C in the subject is reduced about 20% to about 80%, about 30% to about 70%, about 40% to about 70% or about 50% to about 70% after administering the loading dose.

In one aspect, the reduced LDL-C level in the subject increases less than about 10% for at least about 2 weeks after administering the loading dose. In one aspect, the reduced LDL-C level in the subject increases less than about 10%, less than about 5% or less than about 2% for at least about 2 weeks, at least about 3 weeks, at least about 4 weeks, at least about 5 weeks, and up to about 6 weeks after administering the loading dose. In one aspect, the reduced LDL-C level in the subject increases less than about 10% for at least about 2 weeks after administering the maintenance dose. In one aspect, the reduced LDL-C level in the subject increases less than about 10%, less than about 5% or less than about 2% for at least about 2 weeks, at least about 3 weeks, at least about 4 weeks, or at least about 5 weeks, and up to about 6 weeks after administering the maintenance dose. In one aspect, a plasma level of LDL-C in the subject is reduced. In one aspect, a serum level of LDL-C in the subject is reduced.

In one aspect, the subject has a serum low-density lipoprotein cholesterol (LDL-C) level of at least about 100 mg/dL, at least about 110 mg/dL, at least about 120 mg/dL, at least about 130 mg/dL, at least about 140 mg/dL, at least about 150 mg/dL, at least about 160 mg/dL, at least about 170 mg/dL, at least about 180 mg/dL, at least about 190 mg/dL, at least about 200 mg/dL, at least about 300 mg/dL or at least about 400 mg/dL prior to administration of the loading dose.

In one aspect, the subject is a human.

In one aspect, a dosing regimen is provided for reducing a level of PCSK9 in a subject. In one aspect, the dosing regimen includes: administering to the subject a loading dose that include from about 10 mg to about 120 mg of a PCSK9 antisense oligonucleotide of Formula I (SEQ ID NO: 1), as shown in FIG. 1-3, or a pharmaceutically acceptable salt thereof. In one aspect, the level of PCSK9 in the subject is reduced about 10% to about 95% after administering the loading dose to provide a reduced PCSK9 level in the subject. In one aspect, the dosing regimen includes administering one or more maintenance doses that include from about 10 mg to about 120 mg of a PCSK9 antisense oligonucleotide of Formula I (SEQ ID NO: 1), as shown in FIG. 1-3, or a pharmaceutically acceptable salt thereof. In one aspect, the loading dose includes a greater amount of a PCSK9 antisense oligonucleotide of Formula I (SEQ ID NO: 1) or a pharmaceutically acceptable salt thereof than the maintenance dose. In one aspect, the loading dose includes a same amount of a PCSK9 antisense oligonucleotide of Formula I (SEQ ID NO: 1) or a pharmaceutically acceptable salt thereof as the maintenance dose.

In one aspect, a dosing regimen is provided for reducing a level of PCSK9 in a subject. In one aspect, the dosing regimen includes: administering to the subject a loading dose that includes a unit dosage that includes from about 10 mg to about 120 mg of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide. In one aspect, the level of PCSK9 in the subject is reduced about 10% to about 95% after administering the loading dose to provide a reduced PCSK9 level in the subject. In one aspect, the dosing regimen includes administering one or more maintenance doses that include a unit dosage that includes from about 10 mg to about 120 mg of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide. In one aspect, the loading dose includes a greater amount that a maintenance dose of the PCSK9 antisense oligonucleotide that includes a modified oligonucleotide and a conjugate group, or a pharmaceutically acceptable salt thereof. In one aspect, the loading dose includes a same amount as the maintenance dose of a PCSK9 antisense oligonucleotide that includes a modified oligonucleotide and a conjugate group or a pharmaceutically acceptable salt thereof.

In one aspect, the maintenance dose is administered to the subject about every 2 weeks to about every 6 weeks. In one aspect, the maintenance dose is administered to the subject about every 2 weeks, about every 3 weeks, about every 4 weeks, about every 5 weeks or every 6 weeks, for at least about 6 weeks, at least about 12 weeks, at least about 26 weeks, or at least about 52 weeks. In one aspect, the maintenance dose is administered to the subject once a month for at least about 3 months. In one aspect, the maintenance dose is administered to the subject once a month for at least about 6 months, at least about 9 months, at least about 12 months or at least about 15 months. In one aspect, the maintenance dose is administered to the subject about every 21 days, about every 28 days, or about every 30 days, for at least about 90 days, at least about 120 days, at least about 180 days, or at least about 365 days.

In one aspect, the loading dose, maintenance dose or both are administered by parenteral administration. In one aspect, parenteral administration includes subcutaneous, intravenous, or intramuscular administration. In one aspect, parenteral administration includes subcutaneous administration.

In one aspect, a level of PCSK9 in a subject is reduced at least about 10%, at least about 20%, at least about 30%, at least about 40%, at least about 50%, at least about 60%, or at least about 70% and up to about 80%, or about 90% after administering the loading dose. In one aspect, the level of PCSK9 in the subject is reduced about 10% to about 95%, about 20% to about 80%, about 30% to about 70%, about 40% to about 70% or about 50% to about 70% after administering the loading dose. In one aspect, the reduced PCSK9 level in the subject increases less than about 10%, less than about 5% or less than about 2% for at least about 2 weeks, at least about 3 weeks, at least about 4 weeks, or at least about 5 weeks, and up to about 6 weeks after administering the loading dose. In one aspect, the reduced PCSK9 level in the subject increases less than about 10%, about 5% or about 2% for at least about 2 weeks, at least about 3 weeks, at least about 4 weeks, or at least about 5 weeks, and up to about 6 weeks after administration of the maintenance dose. In one aspect, a plasma level of PCSK9 in the subject is reduced. In one aspect, a serum level of PCSK9 in the subject is reduced. In one aspect, PCSK9 expression is reduced.

In one aspect, the dosing regimen includes administering 2 or more maintenance doses. In one aspect, the dosing regimen includes administering 3 or more, 4 or more, 5 or more, 6 or more, 9 or more, 12 or more, or 15 or more maintenance doses. In one aspect, each maintenance dose is administered about every 3 weeks to about every 6 weeks after the previous dose. In one aspect, the previous dose is a loading dose. In one aspect, the previous dose is a previous maintenance dose. In one aspect, the maintenance dose is administered for at least about 3 months, about 6 months, or about 1 year.

In one aspect, the reduced LDL-C level in the subject increases less than about 10%, less than about 5% or less than about 2% up to about 6 weeks, about 9 weeks, or about 12 weeks after administration of a maintenance dose. In one aspect, the reduced PCSK9 level in the subject increases less than about 10%, about 5% or about 2% up to about 6 weeks, about 9 weeks, or about 12 weeks after administration of a maintenance dose.

In one aspect, the subject is a human.

In one aspect, a single dose container is provided that includes from about 10 mg to about 120 mg of a PCSK9 antisense oligonucleotide of formula I (SEQ ID NO:1) or a pharmaceutically acceptable salt thereof.

In one aspect, single dose container is provided that includes from about 10 mg to about 120 mg of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide. In one aspect, single dose container includes a single-dose prefilled syringe.

### BRIEF DESCRIPTION OF THE DRAWINGS

FIG. 1 is a schematic of a chemical structure of a PCSK9 antisense oligonucleotide of Formula I as described herein.

FIG. 2 is a schematic of a chemical structure of an unprotonated PCSK9 antisense oligonucleotide of Formula I as described herein.

FIG. 3 is a schematic of a sodium salt of a PCSK9 antisense oligonucleotide of Formula I as described herein.

FIG. 4 is a graph showing the LDL change from baseline (%) for Repatha® dosed twice monthly.

FIG. 5 is a graph showing the impact of a missed dose on the LDL change from baseline (%) for Inclisiran® and AZD8233.

FIG. 6 is a graph showing the Relative Risk Reduction (RRR) of Inclisiran® compared to AZD8233.

FIG. 7 is a graph showing the plasma PCSK9 change in humans with elevated LDL-C levels upon single dosing of AZD8233. FIG. 7A illustrates the measured data and FIG. 7B illustrates the baseline-corrected data at logarithmic scale (N=6 per treated group and N=14 for placebo; Error bars: SEM)

FIG. 8 is a graph showing the LDL-C change in humans from baseline (%) for AZD8233 in a single ascending dose (SAD) study.

FIG. 9 is a graph showing simulated LDL steady state profiles for Inclisiran® compared to AZD8233.

FIG. 10A is a graph showing the plasma PCSK9 change in humans with elevated LDL-c levels upon multiple dosing of AZD8233 at 30 mg (MAD study) at days 1, 8, 29, and 57. FIG. 10B illustrates the LDL-c change from baseline (%) with multiple dosing of AZD8233 at 30 mg (MAD study) at days 1, 8, 29, and 57. Mean of observation of 30 mg MAD cohort with indication of number of patients per timepoint. Not placebo adjusted data. Shaded area represents 90% CI and the line represents median based on 200 clinical trial simulations with 8 subjects per arm with PCSK9-LDL model based on SAD PCSK9 data and historical LDL-C˜PCSK9 relationship.

FIG. 11 shows simulations of LDL-C change from baseline (%) profiles for Repatha® (420 mg monthly) versus AZD8233 (50 mg monthly). AZD8233 showed approximately a 70% reduction in LDL-C from baseline vs. Repatha® which showed approximately a 55% reduction in LDL-C from baseline.

FIG. 12 shows simulations of LDL-C reduction after one missed dose with Repatha® administered twice monthly at 140 mg (LDL-C reduction approximately 37%) vs AZD8233 administered monthly at 50 mg (LDL-C reduction approximately 60%) and time to LDL-C outside of target range (>55 mg/dL) when not adherent.

FIG. 13 shows the change in circulating LDL-C in humans with elevated LDL-C levels upon single dosing of AZD8233. FIG. 13A illustrates the measured data and FIG. 13B illustrates the baseline-corrected data (N=6 per treated group and N=14 for placebo; Error bars: SEM)

FIG. 14A illustrates the baseline-corrected data for circulating PCSK9 levels and FIG. 14B illustrates the baseline-corrected data for LDL-C levels (N=29-30 per treated group and N=30 for placebo). At a dose of 50 mg per month, circulating PCSK9 levels were reduced by 88% at week 12 (95% CI −91, −84) and circulating LDL-C levels were reduced by 72% at week 12 (95% CI −78, −65).

### DETAILED DESCRIPTION

### Definitions

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Active agent” refers to a compound that has measurable specified or selected physiologic activity when administered to a subject in a pharmaceutically effective amount. An active agent can be a therapeutic, prophylactic, or diagnostic agent. In one aspect, “active agent” refers to an antisense oligonucleotide. In one aspect, “active agent” refers to an antisense oligonucleotide that reduces a level of PCSK9 in a subject. In one aspect, “active agent” refers to as an antisense oligonucleotide that reduces a level of LDL-C in a subject.

“Administration” or “administering” refers to methods by which a compound or composition is introduced into an individual. In one aspect, administration includes, but is not limited to parenteral administration, such as subcutaneous, intravenous, or intramuscular injection or infusion. In one aspect, administration occurs after the onset of the disease or symptoms, for example, when a disease, or symptoms thereof, are being treated. In one aspect, administration occurs before the onset of the disease or symptoms thereof, for example, when a disease or symptoms thereof are being prevented.

“Amelioration” refers to an improvement or lessening of at least one symptom associated with a disease, disorder, or condition. In one aspect, amelioration includes delaying or slowing the progression or severity at least one symptom associated with a disease, disorder or condition. Disease progression can be determined by subjective or objective measures, which are known to those skilled in the art.

“Antisense oligonucleotide” (ASO) refers to an oligonucleotide that targets a transcript of a gene to reduce expression of the gene product. In one aspect, the antisense oligonucleotide includes an oligonucleotide and one or more additional features, such as a conjugate group or a terminal group. In one aspect, the antisense oligonucleotide includes a single-stranded or double-stranded oligonucleotide. In one aspect, the antisense oligonucleotide includes a sequence that is complementary to a target nucleic acid and reduces target nucleic acid levels as compared to target nucleic acid levels in the absence of the antisense oligonucleotide. In one aspect, the antisense oligonucleotide inhibits PCSK9 expression. In one aspect, the antisense oligonucleotide reduces the amount of PCSK9 protein in a subject.

“cEt” or “constrained ethyl” refers to a bicyclic furanosyl sugar moiety that includes a bridge connecting the 4′-carbon and the 2′-carbon, in which the bridge has the formula: 4′-CH(CH₃)—O-2′. “cEt nucleoside” refers to a nucleoside that includes a cEt modified sugar moiety.

“Cardiovascular disease” refers to a disease, disorder or condition of the heart and/or circulatory system. In one aspect, “cardiovascular disease” refers to a disease, condition or disorder associated with PCSK9. In one aspect, the cardiovascular disease includes a disease, disorder or condition associated with an elevated level of PCSK9 in a subject. In one aspect, the cardiovascular disease includes a disease, disorder or condition associated with an elevated level of LDL-C in a subject. Examples of cardiovascular disease include, but are not limited to, arteriosclerosis, coronary artery disease, heart valve disease, arrhythmia, heart failure, hypertension, orthostatic hypotension, shock, endocarditis, diseases of the aorta and its branches, disorders of the peripheral vascular system, heart attack, cardiomyopathy, and congenital heart disease. In one aspect, the cardiovascular disease includes hyperlipidemia, dyslipidemia, atherosclerosis, hypercholesterolemia, or combinations thereof.

“Conjugate” or “conjugate group” can be used interchangeably to refer to a group of atoms covalently attached to an oligonucleotide. In one aspect, the conjugate group includes a conjugate moiety and a conjugate linker that attaches the conjugate moiety to the oligonucleotide. In one aspect, the conjugate includes triantennary N-acetyl galctosamine (GalNAc). In one aspect, the conjugate includes trishexylamino (THA) GalNAc. In one aspect, the GalNAc conjugate is linked to the 5′ terminus of an antisense oligonucleotide. In one aspect, the GalNAc conjugate is linked to the 3′ terminus of an antisense oligonucleotide. In one aspect, (THA) GalNAc conjugate is linked to the 5′ terminus of an antisense oligonucleotide. In one aspect, (THA) GalNAc conjugate is linked to the 3′ terminus of an antisense oligonucleotide. In one aspect, the conjugate includes 5′-trishexylamino-(THA)-C₆GalNAC3, linked to the 5′ end of a PCKS9 antisense oligonucleotide.

“Circulating level” refers to the level of a compound, for example PCSK9 or LDL-C that is present in the blood, plasma or serum of a subject. “Serum PCSK9” and “plasma PCSK9” refer to PCSK9 in the serum and plasma, respectively. “Serum LDL-C” and “plasma LDL-C” refer to LDL-C in the serum and plasma, respectively.

“Disease associated with PCSK9” refers to any disease, disorder or condition that is completely or partially caused by or is the result of PCSK9. In one aspect, the disease, disorder or condition is due to aberrant PCSK9 expression. In one aspect, the disease, disorder or condition is due to PCSK9 overexpression. In one aspect, the disease, disorder or condition is due to aberrant upregulation of PCSK9. Disease associated with PCSK9 include, but are not limited to, elevated total cholesterol levels, elevated low-density lipoprotein cholesterol (LDL-C) levels, hyperlipidemia, dyslipidemia, hypercholesterolemia, including, for example, hypercholesterolemia uncontrolled by statins, familial hypercholesterolemia or non-familial hypercholesterolemia, atherosclerosis, cardiovascular diseases, or combinations thereof. In one aspect, the disease or disorder associated with PCSK9 includes elevated low-density lipoprotein cholesterol (LDL-C) levels, hyperlipidemia, dyslipidemia, hypercholesterolemia, or combinations thereof.

“Dosing regimen” refers to specific dosages, sets of unit dosages, timing of dosages, and repeat times for dosages that are administered to a subject. In one aspect, the dosages are separated by a period of time. In one aspect, the dosing regimen includes administering one or more unit dosages to a subject. In one aspect, the dosing regimen includes administering a plurality of unit dosages to a subject, wherein administration of each unit dosage is separated by a specified time period. In one aspect, some unit dosages within a dosing regimen include different amounts of active agent. In one aspect, all unit dosages within a dosing regimen include the same amount of active agent. In one aspect, the dosing regimen includes a loading dose followed by one or more maintenance doses. In one aspect, the dosing regimen includes an initial loading dose, followed by regular monthly administration, e.g., monthly administration, of maintenance doses. In one aspect, the loading dose includes a unit dosage with the same amount of active agent as the unit dosage for one or more maintenance doses. In one aspect, the loading dose includes a unit dosage with a greater amount of active agent than the unit dosage for one or more maintenance doses. In one aspect, the unit dosage for each maintenance dose includes the same amount of active agent. In one aspect, the unit dosage for some of the maintenance doses includes a different amount of active agent than the unit dosage for other maintenance doses During the maintenance period in which regular monthly maintenance doses are administered, each unit dosage may be the same or different. In one aspect, the dosing regimen is used to treat a subject to at least ameliorate one or more symptoms of a disease, disorder or condition, such as a cardiovascular disease. In one aspect, the dosing regimen is used to halt, inhibit or reverse progress of a disease, disorder or condition, such as a cardiovascular disease

“Dose” refers a specified quantity of a compound that is provided in a single administration. In one aspect, a dose may be administered in two or more boluses, for example, if the desired dose requires a volume not easily accommodated by a single administration, for example, a single injection.

“Dosage unit” or “unit dosage” refers to a physically discrete unit containing a predetermined quantity of an active agent such as an antisense oligonucleotide. In one aspect, a “unit dosage” refers to physically discrete units suitable as single dosages to a subject, such as a human. In one aspect, the unit dosage comprises an active agent and a pharmaceutical diluent, carrier or vehicle. In one aspect, a unit dosage is included in a container, such as a vial, ampule or syringe. In one aspect, the unit dosage is included in a single dose container, or alternately in a multi-dose container. In one aspect, the unit dosage is included in single-dose prefilled syringe. In one aspect, the unit dosage is included in an autoinjector device.

“Fixed dose” refers to a dose of a compound that is used for all subjects regardless of specific subject-related factors, such as weight. Typically, a fixed dose is indicated in milligrams (mg). “Weight-based dose” refers to a dose of a compound that changes depending on the weight of the subject. Typically, a weight-based does is indicated in milligrams/kilogram (mg/kg).

“Loading dose” refers to a dose administered at the beginning of a dosing regimen. In some embodiments, the loading dose is administered to achieve a desired condition in a subject, for example, a desired initial concentration of antisense oligonucleotide or desired physiological effect in the subject. In one aspect, the loading dose is a single unit dosage that is administered to a subject at the beginning of a course of treatment. In one aspect, the desired physiological effect is a reduced PCSK9 level in a patient. In one aspect, the desired physiological effect is a reduced LDL-C level in a patient. In one aspect, the loading dose reduces a level of PCSK9 and/or a level of LDL-C from a baseline level (e.g., pre-administration level) to a reduced level.

“Maintenance dose” refers to one or more, or a series of doses that are administered after the loading dose. In some embodiments, the maintenance dose is administered to administered to a subject to maintain the desired condition which results from the loading dose. In one aspect, one or more, or a plurality of single unit maintenance dosages are administered to a subject serially at a specified time period to maintain the desired condition obtained after administering the loading dose. In one aspect, the desired condition includes a desired concentration of antisense oligonucleotide in the subject or desired physiological effect. In one aspect, the desired physiological effect includes a reduced PCSK9 level in a patient. In one aspect, the desired physiological effect includes a reduced LDL-C level in a patient. In one aspect, the maintenance dose reduces a level of PCSK9 and/or a level of LDL-C in a patient. In one aspect, the maintenance dose is administered on a monthly basis. “Monthly” refers to administration of a compound once in about a 21-, 28- or 30-day period, or about once in a calendar month. “Monthly maintenance dose” refers to a regularly administered dosage within about four weeks or about 28 days or about 30 days from the preceding dose. In one aspect, the preceding dose is the loading dose. In one aspect, the preceding dose is a preceding maintenance dose. “Maintenance period” refers to a time period after a desired condition is achieved in a subject during which one or more maintenance doses are administered to the subject.

“Effective amount” refers to an amount of an active agent or compound that is sufficient to effectuate a desired physiological outcome in a subject. The effective amount can vary depending on the health and physical condition of the subject.

“Efficacy” refers to the ability of an active agent or compound to produce a desired effect.

“Excipient” refers to a substance other than the active agent or water that is included in a pharmaceutical formulation, including, but not limited to, a diluent, preservative, or stabilizing agent.

“Expression” includes all the functions by which a gene's coded information is converted into structures present and operating in a cell. Such structures include, but are not limited to, the products of transcription and translation.

“Gapmer” refers to an oligonucleotide with an internal region that has a plurality of nucleosides that support RNase H cleavage that is positioned between external regions having one or more nucleosides, in which the nucleosides of the internal region are chemically distinct from the nucleoside or nucleosides of the external regions. “Gap segment” or “Gap” refers to the plurality of nucleotides that make up the internal region of a gapmer. “Wing segment” or “wing” refers to one or more nucleosides that make up the external region of a gapmer. In one aspect, the “wing segment” includes one or more modified nucleosides, for example, to enhance inhibitory activity, increase binding affinity for a target nucleic acid, or improve resistance to degradation by in vivo nucleases.

“Hybridization” refers to the annealing of oligonucleotides and/or nucleic acids. While not limited to a particular mechanism, the most common mechanism of hybridization involves hydrogen bonding, such as Watson-Crick, Hoogsteen or reversed Hoogsteen hydrogen bonding, between complementary nucleobases. In one aspect, complementary nucleic acid molecules include, but are not limited to, an antisense oligonucleotide and a target nucleic acid.

“Inhibiting expression or activity” refers to a reduction or blockade of expression or activity of a target relative to the expression of activity in an untreated or control sample and does not require total elimination of expression or activity.

“Internucleoside linkage” refers to a group or bond that forms a covalent linkage between adjacent nucleosides in an oligonucleotide. “Modified internucleoside linkage” refers to any internucleoside linkage other than a naturally occurring, phosphate internucleoside linkage. Non-phosphate linkages are referred to herein as modified internucleoside linkages. In one aspect, the modified internucleoside linkage includes a “phosphorothioate linkage,” a modified phosphate linkage in which one of the non-bridging oxygen atoms is replaced with a sulfur atom.

“Low-density lipoprotein-cholesterol” (LDL-C) refers to cholesterol associated with low-density lipoprotein particles. In one aspect, LDL-C concentrations of about 100 mg/dL, from about 100 to about 129 mg/dL, from about 130 to about 159 mg/dL, from about 160 to about 189 mg/dL, and at least 190 mg/dL can be considered optimal, near optimal/above optimal, borderline high, high, and very high, respectively. In one aspect, a desired therapeutic outcome is to reduce LDL-C levels to less than about 190 mg/dL. In one aspect, a desired therapeutic outcome is to reduce LDL-C levels to less than about 160 mg/dL. In one aspect, a desired therapeutic outcome is to reduce LDL-C levels to less than about 130 mg/dL. In one aspect, a desired therapeutic outcome is to reduce LDL-C levels to less than about 100 mg/dL. In one aspect, a desired therapeutic outcome is to reduce LDL-C levels to less than about 70 mg/dL.

“Nucleic acid” refers to molecules made up of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” refers to a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. In one aspect, the modified nucleobase is “5-methylcytosine”, a cytosine with a methyl group attached to the 5 position.

“Nucleobase sequence” refers to the order of contiguous nucleobases in a nucleic acid or oligonucleotide independent of any sugar or internucleoside linkage.

“Nucleoside” refers to a compound including a nucleobase and a sugar moiety. The nucleobase and sugar moiety can each be, independently, unmodified or modified. “Modified nucleoside” refers to a nucleoside including a modified nucleobase and/or a modified sugar moiety.

“Nucleotide” refers to a nucleoside in which a phosphate group is covalently linked to the sugar portion of the nucleoside.

“Oligomeric compound” refers to a compound that includes an oligonucleotide and optionally one or more additional features, including, for example, a conjugate group or terminal group.

“Oligonucleotide” refers to a polymer of linked nucleosides, each of which can be modified or unmodified, independent one from another. “Modified oligonucleotide” refers to an oligonucleotide, wherein at least one sugar, nucleobase, or internucleoside linkage is modified. “Unmodified oligonucleotide” refers to an oligonucleotide that does not include any sugar, nucleobase, or internucleoside modification.

“Parenteral administration” refers to administration through injection or infusion and includes, but is not limited to, subcutaneous administration, intravenous administration, intramuscular administration, intraarterial administration, intraperitoneal administration, and intracranial administration,

“PCSK9” refers to proprotein convertase subtilisin/kexin type 9, an enzyme with an important role in lipoprotein metabolism and to any nucleic acid encoding PCSK9, including, for example, a DNA sequence encoding PCSK9, an RNA sequence transcribed from DNA encoding PCSK9 (including genomic DNA including introns and exons) or an mRNA sequence encoding PCSK9. In one aspect, PCSK9 is human PCSK9.

“PCSK9 inhibitor” refers to an active agent capable of specifically inhibiting PCSK9 expression or activity or reducing a level of PCSK9 in a subject. “PCSK9 antisense oligonucleotide” is an antisense oligonucleotide that is capable of inhibiting PCSK9 expression or activity or reducing a level of PCSK9 in a subject. In one aspect, the PCSK9 antisense oligonucleotide includes a nucleobase sequence of at least 8, 9, 10, 11, 12, 13, 14, 15 or 16 contiguous nucleobases of the sequence in SEQ ID NO: 1. In one aspect, the PCSK9 antisense oligonucleotide includes a nucleobase sequence of SEQ ID NO: 1. In one aspect, the PCSK9 antisense oligonucleotide has a nucleobase sequence consisting of SEQ ID NO: 1. In one aspect, the PCSK9 antisense oligonucleotide has a chemical structure of Formula I, or a pharmaceutically acceptable salt thereof. In one aspect, the PCSK9 antisense oligonucleotide includes a modified oligonucleotide and a conjugate group, or a pharmaceutically acceptable salt thereof. In one aspect the modified oligonucleotide consists of SEQ ID NO:1 and includes a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment comprises a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

“Prevent” refers to delaying or forestalling the onset, development or progression of a disease, disorder, or condition for a period of time. In one aspect, the term “prevent” refers to total or partial inhibition of the development, recurrence, onset or spread of a disease or disorder associated with PCSK9 and/or a symptom related thereto.

“Reduce” means to bring down to a smaller extent, size, amount, or number. “Reduced level” refers to a level that is reduced as compared to a level prior to administration of an active agent.

“Reduced PCSK9 level” refers to a reduced level of PCSK9 in a subject achieved after administration of an initial loading dose as well as the reduced level of PCSK9 maintained after administration of one or more maintenance doses.

“Reduced LDL-C level” refers to a reduced level of LDL-C in a subject achieved after the administration of an initial loading dose as well as the reduced level of LDL-C maintained after administration of one or more maintenance doses.

“Relative Risk Reduction” refers to the extent to which the risk of a poor outcome is reduced by an intervention, for example, the extent to which a subject is at risk for developing a cardiovascular disease or symptoms of cardiovascular disease, or dying from a cardiovascular disease is reduced.

“Subject” and “patient” can be used interchangeably to refer to a mammal, for example, a human, that has a disease or one or more symptoms of a disease associated with PCSK9. In one aspect, the subject is a mammal, for example a human, at risk of developing a disease or one or more symptoms of a disease associated with PCSK9.

“Target nucleotide sequence” refers to a contiguous portion of a nucleotide sequence of PCSK9. In one aspect, the target nucleotide sequence is a DNA sequence. In one aspect, the target nucleotide sequence is an RNA sequence. In one aspect, the target nucleotide sequence is mRNA.

“Therapeutically effective amount” refers to an amount of an active agent, for example, an antisense oligonucleotide, that provides a therapeutic benefit to a subject.

“Treat”, “treatment”, and “treating” are used interchangeably to refer to administering an active agent to a subject to effect an alteration or improvement of a disease, disorder, or condition in the subject. In one aspect, “treating” refers to a reduction or amelioration of the progression, severity, and/or duration of a disease associated with PCSK9 resulting from administration of one or more therapies.

### Overview

Described herein are unit dosages, dosing regimens and methods for treating a disease associated with PCSK9. In one aspect, a unit dosage is provided that includes a PCSK9 inhibitor. In one aspect, a dosing regimen is provided for treating a disease associated with PCSK9, in which the dosing regimen includes administering a PCSK9 inhibitor to a subject. In one aspect, a method is provided for treating a disease associated with PCSK9, in which the method includes administering a PCSK9 inhibitor to a subject. In one aspect, the PCSK9 inhibitor is a PCSK9 antisense oligonucleotide. Antisense oligonucleotides that inhibit PCSK9 expression, which may be useful for treating, preventing or ameliorating a disease associated with PCSK9 are disclosed in U.S. Pat. No. 10,517,953, issued Dec. 31, 2019, entitled “MODULATORS OF PCSK9 EXPRESSION”, the disclosure of which is hereby incorporated by reference in its entirety.

In one aspect, a unit dosage is provided that includes a compound for reducing an amount or activity of PCSK9 mRNA in a subject. In one aspect, a unit dosage is provided that includes a PCSK9 antisense oligonucleotide for reducing an amount or activity of PCSK9 mRNA in a subject. In one aspect, a dosing regimen is provided for reducing an amount or activity of PCSK9 mRNA in a subject. In one aspect, a method is provided for reducing an amount or activity of PCSK9 mRNA in a subject.

In one aspect, a unit dosage is provided that includes a compound for reducing a level PCSK9 expression in a subject. In one aspect, a unit dosage is provided that includes a PCSK9 antisense oligonucleotide for reducing a level PCSK9 expression in a subject. In one aspect, a dosing regimen is provided for reducing a level of PCSK9 expression in a subject. In one aspect, a method is provided for reducing a level of PCSK9 expression in a subject.

In one aspect, a unit dosage is provided that includes a compound for reducing a level of circulating PCSK9 in a subject. In one aspect, a unit dosage is provided that includes a PCSK9 antisense oligonucleotide for reducing a level of circulating PCSK9 in a subject. In one aspect, a dosing regimen is provided for reducing a level of circulating PCSK9 in a subject. In one aspect, a method is provided for reducing a level of circulating PCSK9 in a subject. In one aspect, circulating PCSK9 includes plasma PCSK9. In one aspect, circulating PCSK9 includes serum PCSK9.

In one aspect, a unit dosage is provided for reducing a level of LDL-C in a subject. In one aspect, unit dosage is provided for reducing a circulating level of LDL-C in a subject. In one aspect, circulating LDL-C includes serum LDL-C. In one aspect, circulating LDL-C includes plasma LDL-C.

In one aspect, the unit dosage includes a compound that is a PCSK9 inhibitor. In one aspect, the unit dosage includes a compound that is an antisense compound. In one aspect, the unit dosage includes a compound that is an antisense oligonucleotide (ASO). In one aspect, the unit dosage includes a compound that is a PCSK9 antisense oligonucleotide. In one aspect, the PCSK9 antisense oligonucleotide includes a modified oligonucleotide. In one aspect, the PCSK9 antisense oligonucleotide includes a single-stranded oligonucleotide. In one aspect, the PCSK9 antisense oligonucleotide includes a double stranded oligonucleotide. In one aspect, the PCSK9 antisense oligonucleotide includes deoxyribonucleotides. In one aspect, the PCSK9 antisense oligonucleotide includes ribonucleotides.

In one aspect, a unit dosage, dosing regimen or method of treatment is provided for treating, preventing, ameliorating, or slowing progression of a cardiovascular disease in an animal. In one aspect, the disease includes dyslipidemia. In one aspect, the disease includes mixed dyslipidemia. In one aspect, the disease includes hypercholesterolemia.

In one aspect, a dosing regimen is provided. In one aspect, the dosing regimen reduces an amount or activity of PCSK9 in a subject. In one aspect, the dosing regimen reduces PCSK9 expression in a subject. In one aspect, the dosing regimen reduces a level of circulating PCSK9 in a subject. In one aspect, circulating PCSK9 includes plasma PCSK9. In one aspect, circulating PCSK9 includes serum PCSK9.

In one aspect, a dosing regimen is provided for reducing a level of LDL-C in a subject. In one aspect, the dosing regimen reduces a circulating level of LDL-C in a subject. In one aspect, circulating LDL-C includes serum LDL-C. In one aspect, circulating LDL-C includes plasma LDL-C.

In one aspect, a dosing regimen is provided that includes a monthly dosing regimen of a unit dosage of a PCSK9 antisense oligonucleotide to obtain a reduced level of PCSK9 in a subject. In one aspect, a dosing regimen is provided that includes a monthly dosing regimen of a unit dosage of a PCSK9 antisense oligonucleotide to obtain a reduced level of LDL-C in a subject.

In one aspect, a method for reducing an amount or activity of PCSK9 in a subject is provided. In one aspect, the method reduces PCSK9 expression in a subject. In one aspect, the method reduces a level of circulating PCSK9 in a subject. In one aspect, circulating PCSK9 includes plasma PCSK9. In one aspect, circulating PCSK9 includes serum PCSK9.

In one aspect, a method is provided for reducing a level of LDL-C in a subject. In one aspect, the method reduces a circulating level of LDL-C in a subject. In one aspect, circulating LDL-C includes serum LDL-C. In one aspect, circulating LDL-C includes plasma LDL-C.

The present disclosure also relates to dosing regimens or methods that can be carried out either in a clinical setting or at the home of a patient. In at least one embodiment, the present disclosure relates to dosing regimens or methods that can be carried out at the home of a patient Additionally, it has been found that, once a reduced level of PCSK9 and/or LDL-C is obtained in a subject, a dose may be missed without significantly increasing the reduced level of PCSK9 and/or LDL-C in the patient. In one aspect, the dosing regimen includes administering a maintenance dose once a month. In one aspect, the dosing regimen includes administering a maintenance dose once a month within a maintenance period. In one aspect, a patient can miss a dose within the monthly dosing regimen. In one aspect, the patient can miss more than one consecutive maintenance dose in a monthly dosing regimen. In one aspect, the patient can one maintenance dose within a 6-month time period of the monthly dosing regimen. In one aspect, the patient can miss up to two maintenance doses within a 6-month time period of the monthly dosing regimen. In one aspect, the patient can miss one maintenance dose within a 12-month time period of the monthly dosing regimen. In one aspect, the patient can miss up to two maintenance doses within a 12-month time period of the monthly dosing regimen. In one aspect, the patient can miss up to two consecutive maintenance doses within a 6-month time period. In one aspect, the patient can miss up to two consecutive maintenance doses within a 12-month time period. In one aspect, the patient can miss up to two non-consecutive doses within a 6-month time period. In one aspect, the patient can miss up to two non-consecutive doses within a 12-month time period.

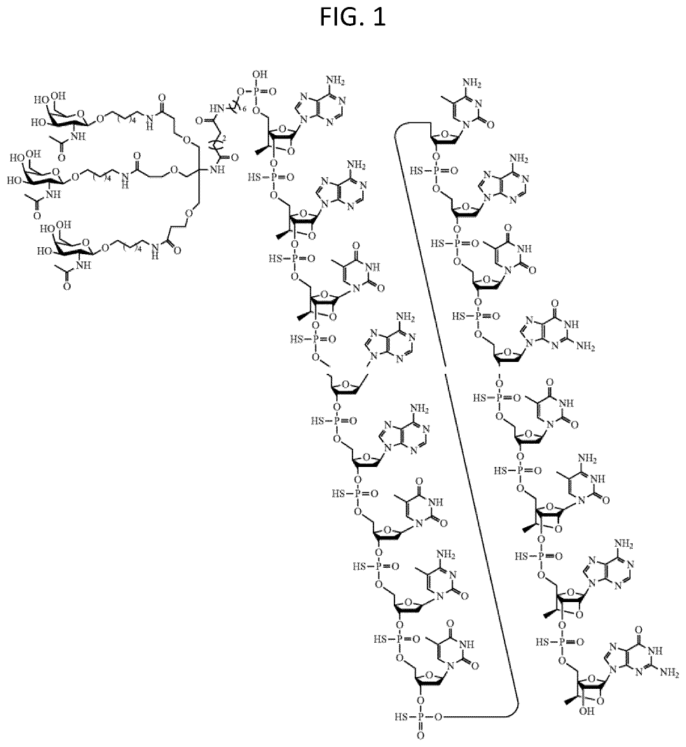

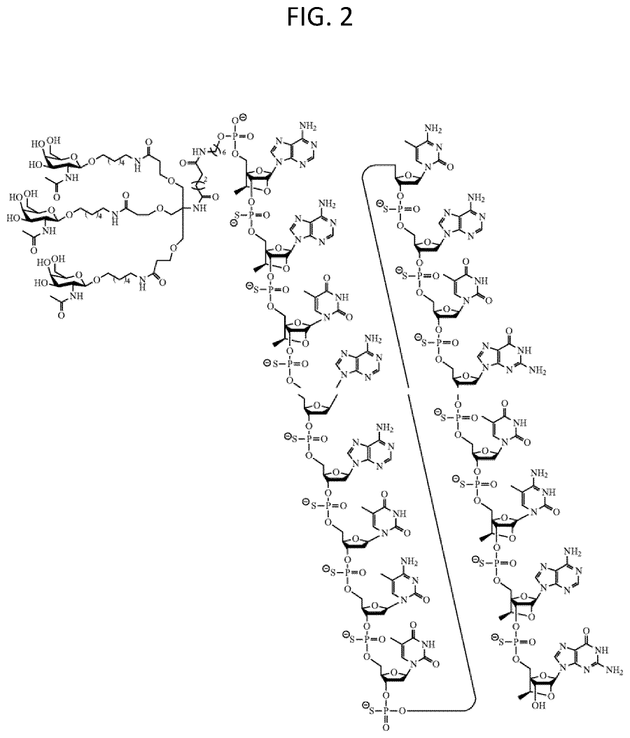

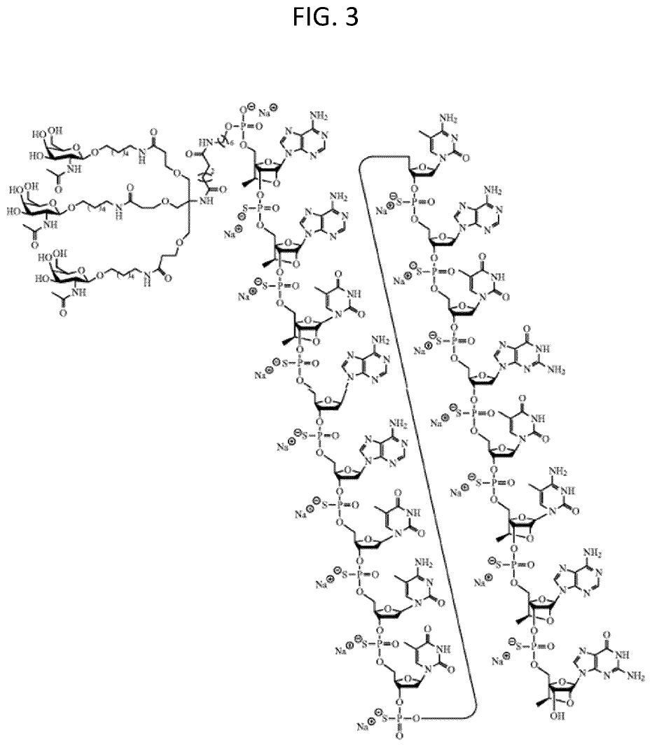

In one aspect, the compound includes a PCSK9 antisense oligonucleotide with a nucleobase sequence of at least 8, 9, 10, 11, 12, 13, 14, 15 or 16 contiguous nucleobases of the sequence in SEQ ID NO: 1. In one aspect, the compound includes a PCSK9 antisense oligonucleotide with a nucleobase sequence of SEQ ID NO: 1. In one aspect, the compound includes a PCSK9 antisense oligonucleotide with a nucleobase sequence consisting of SEQ ID NO: 1. In one aspect, the PCSK9 antisense oligonucleotide has a chemical structure of Formula I, or a pharmaceutically acceptable salt thereof. The protonated form of the compound of Formula I is shown in FIG. 1 and the deprotonated form is shown in FIG. 2. The sodium salt of the compound of Formula I is shown in FIG. 3. In one aspect, the pharmaceutically acceptable salt is a sodium salt. In one aspect, the pharmaceutically acceptable salt is a potassium salt.

In one aspect, the PCSK9 inhibitor includes a modified oligonucleotide. In one aspect, the modified oligonucleotide includes at least one modified internucleoside linkage, at least one modified sugar, at least one modified nucleobase, or a combination thereof. In one aspect, the modified oligonucleotide includes at least one modified internucleotide linkage. In one aspect, the modified oligonucleotide includes a phosphorothioate internucleoside linkage.

In one aspect, the PCSK9 inhibitor is a PCSK9 antisense oligonucleotide that includes a modified oligonucleotide that includes:
a gap segment consisting of ten linked deoxynucleosides;
a 5′ wing segment consisting of three linked nucleosides; and
a 3′ wing segment consisting of three linked nucleosides.

In one aspect, the PCSK9 inhibitor is a PCSK9 antisense oligonucleotide that includes a modified oligonucleotide and a conjugate group, or a pharmaceutically acceptable salt thereof. In one aspect, the conjugate group is linked to the modified oligonucleotide at the 5′ end of the modified oligonucleotide. In one aspect, the conjugate group is linked to the modified oligonucleotide at the 3′ end of the modified oligonucleotide. In one aspect, the conjugate group includes at least one N-Acetylgalactosamine (GalNAc), at least two N-Acetylgalactosamines (GalNAcs), or at least three N-Acetylgalactosamines (GalNAcs).

In one aspect, the PCSK9 inhibitor includes a modified oligonucleotide and a conjugate group, wherein the modified oligonucleotide consists of SEQ ID NO:1 and includes:

a gap segment consisting of ten linked deoxynucleosides;
a 5′ wing segment consisting of three linked nucleosides; and
a 3′ wing segment consisting of three linked nucleosides,
wherein the gap segment is positioned between the 5′ wing segment and the 3′ wing segment; wherein each nucleoside of each wing segment comprises a 2′-O-ethyl (cEt) sugar; wherein each internucleoside linkage is a phosphorothioate linkage; and wherein each cytosine is a 5-methylcytosine.

PCSK9 Levels

In one aspect, a unit dosage, dosing regimen or method is provided for reducing a level of PCSK9 in a subject. In one aspect, the PCSK9 nucleic acid has the sequence set forth in RefSeq Accession No. NM 174936.3 (disclosed herein as SEQ ID NO: 2).

As used herein, the term “reducing a level of PCSK9” refers to reducing the level of PCSK9 protein and/or PCSK9 RNA. In one aspect, a level of PCSK9 nucleic acid is reduced in a subject. In one aspect, a level of PCSK9 protein is reduced in a subject. In one aspect, a circulating level of PCSK9 protein is reduced. In one aspect, a serum PCSK9 protein level is reduced. In one aspect, a plasma PCSK9 protein level is reduced.

A reduced level of PCSK9 can be determined using methods known by one of skill in the art. In one aspect, a level of PCSK9 can be determined by methods including, but not limited to, immunoprecipitation, Western blot analysis (immunoblotting), enzyme-linked immunosorbent assay (ELISA), quantitative protein assays, protein activity assays, immunohistochemistry, immunocytochemistry or fluorescence-activated cell sorting (FACS). Antibodies useful for detection of PCSK9 can be obtained from a variety of sources or can be prepared using methods known in the art.

In one aspect, administering a unit dosage of a PCSK9 antisense oligonucleotide results in a reduction of PCSK9 activity or amount about 10% to about 95% as compared to an activity or amount of PCSK9 before administration of the unit dosage. In one aspect, the activity or amount of PCSK9 is reduced at least about 10%, about 20%, about 30%, about 40%, about 50%, about 60%, or about 70% and up to about 80%, or about 90% after administration of the unit dosage as compared to the activity or amount of PCSK9 in the subject before administration of the unit dosage. In one aspect, the activity or amount of PCSK9 is reduced at least about 10%. In one aspect, the activity or amount of PCSK9 is reduced at least about 20%. In one aspect, the activity or amount of PCSK9 is reduced at least about 30%. In one aspect, the activity or amount of PCSK9 is reduced at least about 40%. In one aspect, the activity or amount of PCSK9 is reduced at least or about 50%. In one aspect, the activity or amount of PCSK9 is reduced by at least about 60%. In one aspect, the activity or amount of PCSK9 is reduced at least about 70%. In one aspect, the activity or amount of PCSK9 is reduced up to about 80%. In one aspect, the activity or amount of PCSK9 is reduced up to about 90%.

In one aspect, administering a unit dosage of a PCSK9 antisense oligonucleotide results in reduction PCSK9 expression from about 10% to about 95% as compared to PCSK9 expression before administration of the unit dosage. In one aspect expression of PCSK9 is reduced at least about 10%. In one aspect, expression of PCSK9 is reduced at least about 20%. In one aspect, expression of PCSK9 is reduced at least about 30%. In one aspect, expression of PCSK9 is reduced at least about 40%. In one aspect, expression of PCSK9 is reduced at least about 50%. In one aspect, expression of PCSK9 is reduced at least about 60%. In one aspect, expression of PCSK9 is reduced at least about 70%. In one aspect, expression of PCSK9 is reduced up to about 80%. In one aspect, expression of PCSK9 is reduced up to about 90%.

In one aspect, administering a unit dosage of a PCSK9 antisense oligonucleotide results in a circulating PCSK9 level that is reduced about 10% to about 95% as compared to a circulating level of PCSK9 before administration of the unit dosage. In one aspect, the circulating level of PCSK9 is reduced at least about 10%. In one aspect, the circulating level of PCSK9 is reduced at least about 20%. In one aspect, the circulating level of PCSK9 is reduced at least about 30%. In one aspect, the circulating level of PCSK9 is reduced at least about 40%. In one aspect, the circulating level of PCSK9 is reduced at least or about 50%. In one aspect, the circulating level of PCSK9 is reduced at least about 60%. In one aspect, the circulating level of PCSK9 is reduced at least about 70%. In one aspect, the circulating level of PCSK9 is reduced up to about 80%. In one aspect, the circulating level of PCSK9 is reduced up to or about 90%.

LDL-C Levels

In one aspect, administering a unit dosage of a PCSK9 antisense oligonucleotide results in a reduction in a level of LDL-C in a subject. In one aspect, administering a unit dosage of a PCSK9 antisense oligonucleotide results in a reduction in a circulating level of LDL-C in a subject. In one aspect, administering a unit dosage of a PCSK9 antisense oligonucleotide results in a reduction in a level of LDL-C in the plasma of the subject. In one aspect, administering a unit dosage of a PCSK9 antisense oligonucleotide results in a reduction in a level of LDL-C in the serum of the subject. Methods for determining plasma and serum concentrations of LDL-C are known.

In some embodiments, the unit dosage form of the present disclosure is administered to a subject in need thereof. In one aspect, a unit dosage of a PCSK9 antisense oligonucleotide is administered to a subject having a serum low-density lipoprotein cholesterol (LDL-C) level of at least about 100 mg/dL. In one aspect, a unit dosage of a PCSK9 antisense oligonucleotide is administered to a subject having a serum low-density lipoprotein cholesterol (LDL-C) level of at least about 110 mg/dL. In one aspect, a unit dosage of a PCSK9 antisense oligonucleotide is administered to a subject having a serum low-density lipoprotein cholesterol (LDL-C) level of at least about 120 mg/dL. In one aspect, a unit dosage of a PCSK9 antisense oligonucleotide is administered to a subject having a serum low-density lipoprotein cholesterol (LDL-C) level of at least about 130 mg/dL. In one aspect, a unit dosage of a PCSK9 antisense oligonucleotide is administered to a subject having a serum low-density lipoprotein cholesterol (LDL-C) level of at least about 140 mg/dL. In one aspect, a unit dosage of a PCSK9 antisense oligonucleotide is administered to a subject having a serum low-density lipoprotein cholesterol (LDL-C) level of at least about 150 mg/dL. In one aspect, a unit dosage of a PCSK9 antisense oligonucleotide is administered to a subject having a serum low-density lipoprotein cholesterol (LDL-C) level of at least about 160 mg/dL. In one aspect, a unit dosage of a PCSK9 antisense oligonucleotide is administered to a subject having a serum low-density lipoprotein cholesterol (LDL-C) level of at least about 170 mg/dL. In one aspect, a unit dosage of a PCSK9 antisense oligonucleotide is administered to a subject having a serum low-density lipoprotein cholesterol (LDL-C) level of at least about 180 mg/dL. In one aspect, a unit dosage of a PCSK9 antisense oligonucleotide is administered to a subject having a serum low-density lipoprotein cholesterol (LDL-C) level of at least about 190 mg/dL. In one aspect, a unit dosage of a PCSK9 antisense oligonucleotide is administered to a subject having a serum low-density lipoprotein cholesterol (LDL-C) level of at least about 200 mg/dL. In one aspect, a unit dosage of a PCSK9 antisense oligonucleotide is administered to a subject having a serum low-density lipoprotein cholesterol (LDL-C) level of at least about 300 mg/dL. In one aspect, a unit dosage of a PCSK9 antisense oligonucleotide is administered to a subject having a serum low-density lipoprotein cholesterol (LDL-C) level of at least about 400 mg/dL prior to administration of the unit dosage.

In one aspect, the level of LDL-C in the subject is reduced about 10% to about 90% after administration of the unit dosage as compared to a level of LDL-C in the subject before administration of the unit dosage. In one aspect, the level of LDL-C in the subject is reduced about 10%. In one aspect, the level of LDL-C in the subject is reduced about 20%. In one aspect, the level of LDL-C in the subject is reduced about 30%. In one aspect, the level of LDL-C in the subject is reduced about 40%. In one aspect, the level of LDL-C in the subject is reduced about 50%. In one aspect, the level of LDL-C in the subject is reduced about 60%. In one aspect, the level of LDL-C in the subject is reduced about 70%. In one aspect, the level of LDL-C in the subject is reduced up to about 80%. In one aspect, the level of LDL-C in the subject is reduced up to about 90%. In one aspect, the level of LDL-C in the subject is reduced about 20% to about 80%. In one aspect, the level of LDL-C in the subject is reduced about 30% to about 70%. In one aspect, the level of LDL-C in the subject is reduced about 40% to about 70%. In one aspect, the level of LDL-C in the subject is reduced about 50% to about 70%.

Unit Dosage

In one aspect, a unit dosage is provided that includes an active agent that reduces a level of PCSK9 in a subject. In one aspect, the unit dosage includes an active agent that reduces a level of PCSK9 expression in a subject. In one aspect, the unit dosage includes an active agent that reduces a circulating level of PCSK9 in a subject. In one aspect, the unit dosage includes an active agent that reduces a serum level of PCSK9 in a subject. In one aspect, the unit dosage includes an active agent that reduces a plasma level of PCSK9 in a subject. In one aspect, the active agent is a PCSK9 antisense oligonucleotide as described herein.

In one aspect, the unit dosage includes a PCSK9 antisense oligonucleotide of Formula I (shown in FIG. 1-3) or a pharmaceutically acceptable salt thereof. In one aspect, the pharmaceutically acceptable salt is a sodium salt. In one aspect, the pharmaceutically acceptable salt is a potassium salt. In one aspect, the unit dosage includes a pharmaceutically acceptable carrier, adjuvant or excipient. In one aspect, the unit dosage includes a PCSK9 antisense oligonucleotide with a nucleobase sequence of SEQ ID NO: 1. In one aspect, PCSK9 antisense oligonucleotide has a nucleobase sequence consisting of SEQ ID NO: 1.

In one aspect, the unit dosage includes from about 10 mg to about 120 mg of a PCSK9 antisense oligonucleotide. In one aspect, the unit dosage includes from about 10 mg to about 120 mg of a PCSK9 antisense oligonucleotide of Formula I, or a pharmaceutically acceptable salt thereof. In one aspect, the unit dosage includes from about 10 mg to about 120 mg of a PCSK9 antisense oligonucleotide with a nucleobase sequence of SEQ ID NO: 1. In one aspect, the unit dosage includes from about 10 mg to about 120 mg of a PCSK9 antisense oligonucleotide having a nucleobase sequence consisting of the sequence of SEQ ID NO: 1.

In one aspect, the unit dosage includes a PCSK9 antisense oligonucleotide that includes a modified oligonucleotide and a conjugate group, or a pharmaceutically acceptable salt thereof. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

In one aspect, the unit dosage includes from about 10 mg to about 120 mg of a PCSK9 antisense oligonucleotide that includes a modified oligonucleotide and a conjugate group, or a pharmaceutically acceptable salt thereof. In one aspect, the unit dosage includes from about 10 mg to about 120 mg of a modified oligonucleotide that consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

In one aspect, the unit dosage includes at least about 10 mg and up to about 150 mg of the PCSK9 antisense oligonucleotide or pharmaceutically acceptable salt thereof. In one aspect, the unit dosage includes at least about 10 mg and up to about 120 mg of the PCSK9 antisense oligonucleotide or pharmaceutically acceptable salt thereof. In one aspect, the unit dosage includes at least about 12 mg, about 15 mg, about 20 mg, about 25 mg, about 30 mg, about 40 mg, about 50 mg, about 60 mg, about 70 mg, about 80 mg, or about 90 mg, and up to about 100 mg, about 110 mg or about 120 mg of the PCSK9 antisense oligonucleotide or a pharmaceutically acceptable salt thereof. In one aspect, the unit dosage includes at least about 15 mg of the PCSK9 antisense oligonucleotide or a pharmaceutically acceptable salt thereof. In one aspect, the unit dosage includes at least about 20 mg of the PCSK9 antisense oligonucleotide or a pharmaceutically acceptable salt thereof. In one aspect, the unit dosage includes at least about 30 mg of the PCSK9 antisense oligonucleotide or a pharmaceutically acceptable salt thereof. In one aspect, the unit dosage includes at least about 40 mg of the PCSK9 antisense oligonucleotide or a pharmaceutically acceptable salt thereof. In one aspect, the unit dosage includes at least about 50 mg of the PCSK9 antisense oligonucleotide or a pharmaceutically acceptable salt thereof. In one aspect, the unit dosage includes at least about 60 mg of the PCSK9 antisense oligonucleotide or a pharmaceutically acceptable salt thereof. In one aspect, the unit dosage includes at least about 70 mg of the PCSK9 antisense oligonucleotide or a pharmaceutically acceptable salt thereof. In one aspect, the unit dosage includes at least about 80 mg of the PCSK9 antisense oligonucleotide or a pharmaceutically acceptable salt thereof. In one aspect, the unit dosage includes at least about 90 mg of the PCSK9 antisense oligonucleotide or a pharmaceutically acceptable salt thereof. In one aspect, the unit dosage includes up to about 100 mg of the PCSK9 antisense oligonucleotide or a pharmaceutically acceptable salt thereof. In one aspect, the unit dosage includes up to about 110 mg of the PCSK9 antisense oligonucleotide or a pharmaceutically acceptable salt thereof. In one aspect, the unit dosage includes up to about 120 mg of the PCSK9 antisense oligonucleotide or a pharmaceutically acceptable salt thereof. In one aspect, the unit dosage includes less than about 100 mg of the PCSK9 antisense oligonucleotide or a pharmaceutically acceptable salt thereof.

In one aspect, the unit dosage includes about 15 mg of the PCSK9 antisense oligonucleotide or a pharmaceutically acceptable salt thereof. In one aspect, the unit dosage includes about 20 mg of the PCSK9 antisense oligonucleotide or a pharmaceutically acceptable salt thereof. In one aspect, the unit dosage includes about 30 mg of the PCSK9 antisense oligonucleotide or a pharmaceutically acceptable salt thereof. In one aspect, the unit dosage includes about 40 mg of the PCSK9 antisense oligonucleotide or a pharmaceutically acceptable salt thereof. In one aspect, the unit dosage includes about 50 mg of the PCSK9 antisense oligonucleotide or a pharmaceutically acceptable salt thereof. In one aspect, the unit dosage includes about 60 mg of the PCSK9 antisense oligonucleotide or a pharmaceutically acceptable salt thereof. In one aspect, the unit dosage includes about 70 mg of the PCSK9 antisense oligonucleotide or a pharmaceutically acceptable salt thereof. In one aspect, the unit dosage includes about 80 mg of the PCSK9 antisense oligonucleotide or a pharmaceutically acceptable salt thereof. In one aspect, the unit dosage includes about 90 mg of the PCSK9 antisense oligonucleotide or a pharmaceutically acceptable salt thereof. In one aspect, the unit dosage includes about 100 mg of the PCSK9 antisense oligonucleotide or a pharmaceutically acceptable salt thereof. In one aspect, the unit dosage includes about 110 mg of the PCSK9 antisense oligonucleotide or a pharmaceutically acceptable salt thereof. In one aspect, the unit dosage includes about 120 mg of the PCSK9 antisense oligonucleotide or a pharmaceutically acceptable salt thereof.

In one aspect, the unit dosage includes from about 50 mg to about 100 mg of the PCSK9 antisense oligonucleotide or a pharmaceutically acceptable salt thereof. In one aspect, the unit dosage includes about 15 mg, about 50 mg, about 70 mg or about 90 mg of the PCSK9 antisense oligonucleotide or a pharmaceutically acceptable salt thereof.

In one aspect, the unit dosage is formulated for parenteral administration. In one aspect, parenteral administration includes subcutaneous, intramuscular or intravenous administration. In one aspect, the unit dosage is formulated for subcutaneous administration.

In one aspect, the unit dosage includes a pharmaceutically acceptable excipient. In one aspect, the pharmaceutically acceptable excipient is saline.

In one aspect, a unit dosage as described herein is used in the manufacture of a medicament for the reduction of a PCSK9 level in a subject. In one aspect, a unit dosage as described herein is used in the manufacture of a medicament for the reduction of a low-density lipoprotein cholesterol (LDL-C) level in a subject. In one aspect, a unit dosage as described herein is used in the manufacture of a medicament for the treatment of disease associated with PCSK9. In one aspect, the disease associated with PCSK9 is selected from: elevated low-density lipoprotein cholesterol (LDL-C) levels, hyperlipidemia, dyslipidemia, atherosclerosis, cardiovascular disease, hypercholesterolemia, and combinations thereof. In one aspect, the disease associated with PCSK9 is dyslipidemia or hypercholesterolemia.

Dosing Regimen

In one aspect, the present disclosure provides a dosing regimen suitable for administering the unit dosage forms described herein over a period of time. In one aspect, a dosing regimen is provided for reducing the level of PCSK9 in a subject. In one aspect, a dosing regimen is provided for reducing the level of PCSK9 expression in a subject. In one aspect, a dosing regimen is provided for reducing the circulating level of PCSK9 in a subject. In one aspect, a dosing regimen is provided for reducing the serum or plasma level of PCSK9 in a subject. In one aspect, a dosing regimen is provided for reducing the low-density lipoprotein cholesterol (LDL-C) level in a subject. In one aspect, a dosing regimen is provided for reducing the circulating level of LDL-C in a subject. In one aspect, a dosing regimen is provided for reducing the plasma level of LDL-C in a subject. In one aspect, a dosing regimen is provided for reducing the serum level of LDL-C in a subject. In one aspect, a dosing regimen is provided for reducing the level of PCSK9 and the level of LDL-C in a subject.

Dosing Regimens without an Initial Loading Dose

In one aspect, the dosing regimen does not include separate loading and maintenance doses but just one or more doses of a PCSK9 inhibitor to a subject over a period of time. In one aspect, the dosing regimen includes administering to the subject a dose that includes a unit dosage of a PCSK9 antisense oligonucleotide. In one aspect, the unit dosages for each of the doses are the same. In one aspect, the unit dosages for the doses are not all the same. In one aspect, the unit dosages for the doses increase over time. In one aspect, the unit dosages for the doses decrease over time.

In one aspect, the dose is administered by parenteral administration. In one aspect, parenteral administration includes subcutaneous, intravenous, or intramuscular administration. In one aspect, parenteral administration includes subcutaneous administration.

In one aspect, the PCSK9 antisense oligonucleotide is administered as a “fixed dose”, e.g., in which the same dosage amount is used for all subjects regardless of subject-related factors such as weight. In one aspect, the PCSK9 antisense oligonucleotide is administered as a “weight-based” dose, e.g., in which the dosage amount can change depending on the weight of the subject.

In one aspect, the dose is administered at the start of treatment. In one aspect, the dose reduces a level of PCSK9 as compared to a level of PCSK9 before administration of the dose. In one aspect, the dose reduces a level of PCSK9 expression as compared to a level of PCSK9 expression prior to administration of the dose. In one aspect, the dose reduces a level of circulating PCSK9 as compared to a level of circulating PCSK9 expression prior to administration of the dose. In one aspect, the dose reduces a serum or plasma level of PCSK9 in the subject.

In one aspect, the dose reduces a LDL-C level as compared to the LDL-C level prior to administration of the dose. In one aspect, the dose reduces a circulating level of LDL-C as compared to the LDL-C level prior to administration of the dose. In one aspect, the dose reduces a plasma level of LDL-C. In one aspect, the dose reduces a serum level of LDL-C.

In one aspect, one or more doses are administered to the subject during a certain period of time. The number of doses administered during the period, or the duration of the period can vary depend on the desired outcome, for example, reducing a level of PCSK9 in a subject, reducing expression of PCSK9 in a subject, reducing a level of LDL-C in a subject, or reducing one or more symptoms of a disease associated with PCSK9 in a subject. In one aspect, the period includes about 1, about 2, about 3, about 4, about 5, about 6, about 7, about 8, about 9, about 10, about 11, about 12, about 13, about 14, about 15, about 16, about 17, about 18, about 19, or about 20 or more doses. In one aspect, the duration of the period is about 3 months, about 6 months, about 9 months, about 12 months, about 15 months, about 24 months or about 2 years, about 3 years, about 4 years, about 5 years, about 6 years, about 7 years, about 8 years, about 9 years, about 10 years or more, e.g., chronic administration. In one aspect, the period is the lifetime of the subject. In one aspect, the period includes more than one dose and the unit dosages administered during the period are all the same. In one aspect, the unit dosages administered during the period are not all the same. In one aspect, the amount of the PCSK9 inhibitor in the unit dosages administered during the period increase over time. In one aspect, the amount of the PCSK9 inhibitor in the unit dosages administered during the period decrease over time.

In one aspect, the dose is administered to the subject about every 2 weeks to about every 6 weeks. In one aspect, the dose is administered to the subject at an interval of about once a week. In one aspect, the dose is administered to the subject at an interval of about once every two weeks. In one aspect, the dose is administered to the subject at an interval of about once every three weeks. In one aspect, the dose is administered to the subject at an interval of about once every 4 weeks. In one aspect, the dose is administered to the subject at an interval of about once every 5 weeks. In one aspect, the dose is administered to the subject at an interval of about once every 6 weeks.

In one aspect, the dose is administered to the subject about every 2 weeks for at least about 6 weeks. In one aspect, the dose is administered to the subject about every 2 weeks for at least about 12 weeks. In one aspect, the dose is administered to the subject about every 2 weeks for at least about 26 weeks. In one aspect, the dose is administered to the subject about every 2 weeks for at least about 52 weeks. In one aspect, the e dose is administered to the subject about every 3 weeks for at least about 12 weeks. In one aspect, the dose is administered to the subject about every 3 weeks for at least about 26 weeks. In one aspect, the dose is administered to the subject about every 3 weeks for at least about 52 weeks. In one aspect, the dose is administered to the subject about every 4 weeks for at least about 12 weeks. In one aspect, the dose is administered to the subject about every 4 weeks for at least about 26 weeks. In one aspect, the dose is administered to the subject about every 4 weeks for at least about 52 weeks. In one aspect, the dose is administered to the subject about every 5 weeks for at least about 12 weeks. In one aspect, the dose is administered to the subject about every 5 weeks for at least about 26 weeks. In one aspect, the dose is administered to the subject about every 5 weeks for at least about 52 weeks. In one aspect, the dose is administered to the subject about every 6 weeks for at least about 12 weeks. In one aspect, the dose is administered to the subject about every 6 weeks for at least about 26 weeks. In one aspect, the dose is administered to the subject about every 6 weeks for at least about 52 weeks.

In one aspect, administration of a dose is repeated monthly. In one aspect, the dose is administered to the subject once a month for at least about 3 months. In one aspect, the dose is administered to the subject once a month for at least about 6 months. In one aspect, the dose is administered to the subject once a month for at least about 9 months. In one aspect, the dose is administered to the subject once a month for at least about 12 months. In one aspect, the dose is administered to the subject once a month for at least about 15 months. In one aspect, the dose is administered to the subject once a month for at least about 24 months.

In one aspect, administration of a dose is repeated once every two months. In one aspect, the dose is administered to the subject once every two months for at least about 4 months. In one aspect, the dose is administered to the subject once every two months for at least about 8 months. In one aspect, the dose is administered to the subject once every two months for at least about 12 months. In one aspect, the dose is administered to the subject once every two months for at least about 18 months. In one aspect, the dose is administered to the subject once every two months for at least about 24 months. In one aspect, the dose is administered to the subject once every two months for at least about 36 months.

In one aspect, administration of a dose is repeated once about every 21 days. In one aspect, administration of a dose is repeated once about every 28 days. In one aspect, administration of a dose is repeated once about every 30 days.

In one aspect, the dose is administered to the subject about every 21 days for at least about 90 days. In one aspect, the dose is administered to the subject about every 21 days for at least about 120 days. In one aspect, the dose is administered to the subject about every 21 days for at least about 180 days. In one aspect, the dose is administered to the subject about every 21 days for at least about 365 days.

In one aspect, the dose is administered to the subject about every 28 days for at least about 90 days. In one aspect, the dose is administered to the subject about every 28 days for at least about 120 days. In one aspect, the dose is administered to the subject about every 28 days for at least about 180 days. In one aspect, the dose is administered to the subject about every 28 days for at least about 365 days.

In one aspect, the dose is administered to the subject about every 30 days for at least about 90 days. In one aspect, the dose is administered to the subject about every 30 days for at least about 120 days. In one aspect, the dose is administered to the subject about every 30 days for at least about 180 days. In one aspect, the dose is administered to the subject about every 30 days for at least about 365 days.

In one aspect, the dosing regimen includes administering the PCKS9 antisense oligonucleotide as a unit dosage of at least about 10 mg, or at least about 20 mg, or at least about 50 mg and up to about 150 mg. In one aspect, the dosing regimen includes administering the PCKS9 antisense oligonucleotide as a unit dosage of at least about 10 mg, or at least about 20 mg, or at least about 50 mg and up to about 120 mg. In one aspect, the dosing regimen includes administering the PCKS9 antisense oligonucleotide as a unit dosage of at least about 12 mg. In one aspect, the dosing regimen includes administering the PCKS9 antisense oligonucleotide as a unit dosage of at least about 10 mg. In one aspect, the dosing regimen includes administering the PCKS9 antisense oligonucleotide as a unit dosage of at least about 15 mg. In one aspect, the dosing regimen includes administering the PCKS9 antisense oligonucleotide as a unit dosage of at least about 20 mg. In one aspect, the dosing regimen includes administering the PCKS9 antisense oligonucleotide as a unit dosage of at least about 25 mg. In one aspect, the dosing regimen includes administering the PCKS9 antisense oligonucleotide as a unit dosage of at least about 30 mg. In one aspect, the dosing regimen includes administering the PCKS9 antisense oligonucleotide as a unit dosage of at least about 40 mg. In one aspect, the dosing regimen includes administering the PCKS9 antisense oligonucleotide as a unit dosage of at least about 50 mg. In one aspect, the dosing regimen includes administering the PCKS9 antisense oligonucleotide as a unit dosage of at least about 60 mg. In one aspect, the dosing regimen includes administering the PCKS9 antisense oligonucleotide as a unit dosage of at least about 70 mg. In one aspect, the dosing regimen includes administering the PCKS9 antisense oligonucleotide as a unit dosage of at least about 80 mg. In one aspect, the dosing regimen includes administering the PCKS9 antisense oligonucleotide as a unit dosage of at least about 90 mg. In one aspect, the dosing regimen includes administering the PCKS9 antisense oligonucleotide as a unit dosage of up to about 100 mg. In one aspect, the dosing regimen includes administering the PCKS9 antisense oligonucleotide as a unit dosage of up to about 110 mg. In one aspect, the dosing regimen includes administering the PCKS9 antisense oligonucleotide as a unit dosage of up to about 120 mg. In one aspect, the dosing regimen includes administering the PCKS9 antisense oligonucleotide as a unit dosage of up to about 130 mg. In one aspect, the dosing regimen includes administering the PCKS9 antisense oligonucleotide as a unit dosage of up to about 140 mg. In one aspect, the dosing regimen includes administering the PCKS9 antisense oligonucleotide as a unit dosage of up to about 150 mg.

In one aspect, the dosing regimen includes administering the PCSK9 antisense oligonucleotide as a unit dosage of about 10 mg, or at least about 20 mg, or at least about 50 mg, to about 150 mg. In one aspect, the dosing regimen includes administering the PCSK9 antisense oligonucleotide as a unit dosage of about 10 mg to about 120 mg. In one aspect, the dosing regimen includes administering the PCSK9 antisense oligonucleotide as a unit dosage of about 50 mg to about 100 mg. In one aspect, the dosing regimen includes administering the PCSK9 antisense oligonucleotide as a unit dosage of about 50 mg to about 90 mg. In one aspect, the dosing regimen includes administering the PCSK9 antisense oligonucleotide as a unit dosage of about 50 mg to about 60 mg. In one aspect, the dosing regimen includes administering the PCSK9 antisense oligonucleotide as a unit dosage of about 60 mg to about 70 mg. In one aspect, the dosing regimen includes administering the PCSK9 antisense oligonucleotide as a unit dosage of about 70 mg to about 80 mg. In one aspect, the dosing regimen includes administering the PCSK9 antisense oligonucleotide as a unit dosage of about 80 mg to about 90 mg. In one aspect, the dosing regimen includes administering the PCSK9 antisense oligonucleotide as a unit dosage of about 90 mg to about 100 mg. In one aspect, the dosing regimen includes administering the PCSK9 antisense oligonucleotide as a unit dosage of about 50 mg.

In one aspect, the dosing regimen includes administering the PCSK9 antisense oligonucleotide as a unit dosage of about 10 mg. In one aspect, the dosing regimen includes administering the PCSK9 antisense oligonucleotide as a unit dosage of about 15 mg. In one aspect, the dosing regimen includes administering the PCSK9 antisense oligonucleotide as a unit dosage of about 20 mg. In one aspect, the dosing regimen includes administering the PCSK9 antisense oligonucleotide as a unit dosage of about 30 mg. In one aspect, the dosing regimen includes administering the PCSK9 antisense oligonucleotide as a unit dosage of about 40 mg. In one aspect, the dosing regimen includes administering the PCSK9 antisense oligonucleotide as a unit dosage of PCSK9 antisense oligonucleotide of about 50 mg. In one aspect, the dosing regimen includes administering the PCSK9 antisense oligonucleotide as a unit dosage of PCSK9 antisense oligonucleotide of about 60 mg. In one aspect, the dosing regimen includes administering the PCSK9 antisense oligonucleotide as a unit dosage of PCSK9 antisense oligonucleotide of about 70 mg. In one aspect, the dosing regimen includes administering the PCSK9 antisense oligonucleotide as a unit dosage of PCSK9 antisense oligonucleotide of about 80 mg. In one aspect, the dosing regimen includes administering the PCSK9 antisense oligonucleotide as a unit dosage of PCSK9 antisense oligonucleotide of about 90 mg. In one aspect, the dosing regimen includes administering the PCSK9 antisense oligonucleotide as a unit dosage of PCSK9 antisense oligonucleotide of about 100 mg. In one aspect, the dosing regimen includes administering the PCSK9 antisense oligonucleotide as a unit dosage of PCSK9 antisense oligonucleotide of about 110 mg. In one aspect, the dosing regimen includes administering the PCSK9 antisense oligonucleotide as a unit dosage of PCSK9 antisense oligonucleotide of about 120 mg. In one aspect, the dosing regimen includes administering the PCSK9 antisense oligonucleotide as a unit dosage of PCSK9 antisense oligonucleotide of about 130 mg. In one aspect, the dosing regimen includes administering the PCSK9 antisense oligonucleotide as a unit dosage of PCSK9 antisense oligonucleotide of about 140 mg. In one aspect, the dosing regimen includes administering the PCSK9 antisense oligonucleotide as a unit dosage of PCSK9 antisense oligonucleotide of about 150 mg. In one aspect, the antisense oligonucleotide is administered in a unit dosage of about 20 mg, about 50 mg, about 60 mg, about 70 mg, about 80 mg, or about 90 mg.

In one aspect, the LDL-C level in the subject is reduced about 10% to about 95% after administering the dose to provide a reduced LDL-C level in the subject. In one aspect, the level of LDL-C in the subject is reduced at least about 20% after administering the dose. In one aspect, the level of LDL-C in the subject is reduced at least about 30% after administering the dose. In one aspect, the level of LDL-C in the subject is reduced at least about 40% after administering the dose. In one aspect, the level of LDL-C in the subject is reduced at least about 50% after administering the dose. In one aspect, the level of LDL-C in the subject is reduced at least about 60% after administering the dose. In one aspect, the level of LDL-C in the subject is reduced at least about 70% after administering the dose. In one aspect, the level of LDL-C in the subject is reduced up to about 80% after administering the dose. In one aspect, the level of LDL-C in the subject is reduced up to about 90% after administering the dose.

In one aspect, the level of LDL-C in the subject is reduced about 20% to about 80% after administering the dose. In one aspect, the level of LDL-C in the subject is reduced about 30% to about 70% after administering the dose. In one aspect, the level of LDL-C in the subject is reduced about 40% to about 70% after administering the dose. In one aspect, the level of LDL-C in the subject is reduced about 50% to about 70% after administering the dose.

In one aspect, the reduced LDL-C level in the subject increases less than about 10% for at least about 2 weeks after administering the dose. In one aspect, the reduced LDL-C level in the subject increases less than about 5% for at least about 2 weeks after administering the dose. In one aspect, the LDL-C level in the subject increases less than about 2% for at least about 2 weeks after administering the dose.

In one aspect, the reduced LDL-C level in the subject increases less than about 10% for at least about 3 weeks after administering the dose. In one aspect, the reduced LDL-C level in the subject increases less than about 5% for at least about 3 weeks after administering the dose. In one aspect, the LDL-C level in the subject increases less than about 2% for at least about 3 weeks after administering the dose.

In one aspect, the reduced LDL-C level in the subject increases less than about 10% for at least about 4 weeks after administering the dose. In one aspect, the reduced LDL-C level in the subject increases less than about 5% for at least about 4 weeks after administering the dose. In one aspect, the LDL-C level in the subject increases less than about 2% for at least about 4 weeks after administering the dose.

In one aspect, the reduced LDL-C level in the subject increases less than about 10% for at least about 5 weeks after administering the dose. In one aspect, the reduced LDL-C level in the subject increases less than about 5% for at least about 5 weeks after administering the dose. In one aspect, the LDL-C level in the subject increases less than about 2% for at least about 5 weeks after administering the dose.

In one aspect, the reduced LDL-C level in the subject increases less than about 10% for up to about 6 weeks after administering the dose. In one aspect, the reduced LDL-C level in the subject increases less than about 5% for up to about 6 weeks after administering the dose. In one aspect, the LDL-C level in the subject increases less than about 2% for up to about 6 weeks after administering the dose.

In one aspect, the reduced LDL-C level in the subject increases less than about 10% for up to about 9 weeks after administering the dose. In one aspect, the reduced LDL-C level in the subject increases less than about 5% for up to about 9 weeks after administering the dose. In one aspect, the LDL-C level in the subject increases less than about 2% for up to about 9 weeks after administering the dose.

In one aspect, the reduced LDL-C level in the subject increases less than about 10% for up to about 12 weeks after administering the dose. In one aspect, the reduced LDL-C level in the subject increases less than about 5% for up to about 12 weeks after administering the dose. In one aspect, the LDL-C level in the subject increases less than about 2% for up to about 12 weeks after administering the dose.

In one level, a circulating level of LDL-C is reduced. In one aspect, a serum level of LCL-C is reduced. In one aspect, a plasma level of LDL-C in the subject is reduced.

In one aspect, the subject has a serum low-density lipoprotein cholesterol (LDL-C) level of at least about 100 mg/dL prior to administration of the dose. In one aspect, the subject has a serum low-density lipoprotein cholesterol (LDL-C) level of at least about 110 mg/dL prior to administration of the dose. In one aspect, the subject has a serum low-density lipoprotein cholesterol (LDL-C) level of at least about 120 mg/dL prior to administration of the dose. In one aspect, the subject has a serum low-density lipoprotein cholesterol (LDL-C) level of at least about 130 mg/dL prior to administration of the dose. In one aspect, the subject has a serum low-density lipoprotein cholesterol (LDL-C) level of at least about 140 mg/dL prior to administration of the dose. In one aspect, the subject has a serum low-density lipoprotein cholesterol (LDL-C) level of at least about 150 mg/dL prior to administration of the dose. In one aspect, the subject has a serum low-density lipoprotein cholesterol (LDL-C) level of at least about 160 mg/dL prior to administration of the dose. In one aspect, the subject has a serum low-density lipoprotein cholesterol (LDL-C) level of at least about 170 mg/dL prior to administration of the dose. In one aspect, the subject has a serum low-density lipoprotein cholesterol (LDL-C) level of at least about 180 mg/dL prior to administration of the dose. In one aspect, the subject has a serum low-density lipoprotein cholesterol (LDL-C) level of at least about 190 mg/dL prior to administration of the dose. In one aspect, the subject has a serum low-density lipoprotein cholesterol (LDL-C) level of at least about 200 mg/dL prior to administration of the dose. In one aspect, the subject has a serum low-density lipoprotein cholesterol (LDL-C) level of at least about 300 mg/dL prior to administration of the dose. In one aspect, the subject has a serum low-density lipoprotein cholesterol (LDL-C) level of at least about 400 mg/dL prior to administration of the dose.

In one aspect, the subject has a serum low-density lipoprotein cholesterol (LDL-C) level of less than about 70 mg/dL for at least about 2 weeks and up to about 6 weeks after administration of the dose. In one aspect, the subject has a serum low-density lipoprotein cholesterol (LDL-C) level of less than about 80 mg/dL for at least about 2 weeks and up to about 6 weeks after administration of the dose. In one aspect, the subject has a serum low-density lipoprotein cholesterol (LDL-C) level of less than about 90 mg/dL for at least about 2 weeks and up to about 6 weeks after administration of the dose. In one aspect, the subject has a serum low-density lipoprotein cholesterol (LDL-C) level of less than about 100 mg/dL for at least about 2 weeks and up to about 6 weeks after administration of the dose. In one aspect, the subject has a serum low-density lipoprotein cholesterol (LDL-C) level of less than about 110 mg/dL for at least about 2 weeks and up to about 6 weeks after administration of the dose. In one aspect, the subject has a serum low-density lipoprotein cholesterol (LDL-C) level of less than about 120 mg/dL for at least about 2 weeks and up to about 6 weeks after administration of the dose. In one aspect, the subject has a serum low-density lipoprotein cholesterol (LDL-C) level of less than about 130 mg/dL for at least about 2 weeks and up to about 6 weeks after administration of the dose.

In one aspect, a dosing regimen is provided for reducing a level of PCSK9 in a subject. In one aspect, a dosing regimen is provided for reducing PCSK9 expression. In one aspect, a dosing regimen is provided for reducing a circulating level of PCSK9 in a subject. In one aspect, a dosing regimen is provided for reducing a plasma level of PCSK9 in a subject. In one aspect, a dosing regimen is provided for reducing a serum level of PCSK9 in a subject.

In one aspect, the level of PCSK9 in the subject is reduced about 10% to about 95% after administration of a PCSK9 inhibitor described herein. In one aspect, the level of PCSK9 in the subject is reduced about 10% to about 95% after administration of a PCSK9 antisense oligonucleotide described herein.

In one aspect, the level of PCSK9 is reduced at least about 10% after administration of a PCSK9 antisense oligonucleotide. In one aspect, the level of PCSK9 is reduced at least about 20% after administration of a PCSK9 antisense oligonucleotide. In one aspect, the level of PCSK9 is reduced at least about 30% after administration of a PCSK9 antisense oligonucleotide. In one aspect, the level of PCSK9 is reduced at least about 40% after administration of a PCSK9 antisense oligonucleotide. In one aspect, the level of PCSK9 is reduced at least about 50% after administration of a PCSK9 antisense oligonucleotide. In one aspect, the level of PCSK9 is reduced at least about 60% after administration of a PCSK9 antisense oligonucleotide. In one aspect, the level of PCSK9 is reduced at least about 70% after administration of a PCSK9 antisense oligonucleotide. In one aspect, the level of PCSK9 is reduced up to about 80% after administration of a PCSK9 antisense oligonucleotide. In one aspect, the level of PCSK9 is reduced up to about 90% after administration of a PCSK9 antisense oligonucleotide.

In one aspect, the level of PCSK9 in the subject is reduced about 10% to about 95% after administration of the PCSK9 antisense oligonucleotide. In one aspect, the level of PCSK9 in the subject is reduced about 20% to about 80% after administration of the PCSK9 antisense oligonucleotide. In one aspect, the level of PCSK9 in the subject is reduced about 30% to about 70% after administration of the PCSK9 antisense oligonucleotide. In one aspect, the level of PCSK9 in the subject is reduced about 40% to about 70% after administration of the PCSK9 antisense oligonucleotide. In one aspect, the level of PCSK9 in the subject is reduced about 50% to about 70% after administration of the PCSK9 antisense oligonucleotide.

In one aspect, the reduced PCSK9 level in the subject increases less than about 10%, for at least about 2 weeks after administering the PCSK9 antisense oligonucleotide. In one aspect, the reduced PCSK9 level in the subject increases less than about 5%, for at least about 2 weeks after administering the PCSK9 antisense oligonucleotide. In one aspect, the reduced PCSK9 level in the subject increases less than about 2%, for at least about 2 weeks after administering the PCSK9 antisense oligonucleotide.

In one aspect, the reduced PCSK9 level in the subject increases less than about 10%, for at least about 3 weeks after administering the PCSK9 antisense oligonucleotide. In one aspect, the reduced PCSK9 level in the subject increases less than about 5%, for at least about 3 weeks after administering the PCSK9 antisense oligonucleotide. In one aspect, the reduced PCSK9 level in the subject increases less than about 2%, for at least about 3 weeks after administering the PCSK9 antisense oligonucleotide.

In one aspect, the reduced PCSK9 level in the subject increases less than about 10%, for at least about 4 weeks after administering the PCSK9 antisense oligonucleotide. In one aspect, the reduced PCSK9 level in the subject increases less than about 5%, for at least about 4 weeks after administering the PCSK9 antisense oligonucleotide. In one aspect, the reduced PCSK9 level in the subject increases less than about 2%, for at least about 4 weeks after administering the PCSK9 antisense oligonucleotide.

In one aspect, the reduced PCSK9 level in the subject increases less than about 10%, for at least about 5 weeks after administering the PCSK9 antisense oligonucleotide. In one aspect, the reduced PCSK9 level in the subject increases less than about 5%, for at least about 5 weeks after administering the PCSK9 antisense oligonucleotide. In one aspect, the reduced PCSK9 level in the subject increases less than about 2%, for at least about 5 weeks after administering the PCSK9 antisense oligonucleotide.

In one aspect, the reduced PCSK9 level in the subject increases less than about 10%, for up to about 6 weeks after administering the PCSK9 antisense oligonucleotide. In one aspect, the reduced PCSK9 level in the subject increases less than about 5%, for up to about 6 weeks after administering the PCSK9 antisense oligonucleotide. In one aspect, the reduced PCSK9 level in the subject increases less than about 2%, for up to about 6 weeks after administering the PCSK9 antisense oligonucleotide.

In one aspect, the reduced PCSK9 level in the subject increases less than about 10%, for up to about 9 weeks after administering the PCSK9 antisense oligonucleotide. In one aspect, the reduced PCSK9 level in the subject increases less than about 5%, for up to about 9 weeks after administering the PCSK9 antisense oligonucleotide. In one aspect, the reduced PCSK9 level in the subject increases less than about 2%, for up to about 9 weeks after administering the PCSK9 antisense oligonucleotide.

In one aspect, the reduced PCSK9 level in the subject increases less than about 10%, for up to about 12 weeks after administering the PCSK9 antisense oligonucleotide. In one aspect, the reduced PCSK9 level in the subject increases less than about 5%, for up to about 12 weeks after administering the PCSK9 antisense oligonucleotide. In one aspect, the reduced PCSK9 level in the subject increases less than about 2%, for up to about 12 weeks after administering the PCSK9 antisense oligonucleotide.

In one aspect, the level of PCSK9 is reduced at least about 10% after administration of the dose. In one aspect, the level of PCSK9 is reduced at least about 20% after administration of the dose. In one aspect, the level of PCSK9 is reduced at least about 30% after administration of the dose. In one aspect, the level of PCSK9 is reduced at least about 40% after administration of the dose. In one aspect, the level of PCSK9 is reduced at least about 50% after administration of the dose. In one aspect, the level of PCSK9 is reduced at least about 60% after administration of the dose. In one aspect, the level of PCSK9 is reduced at least about 70% after administration of the dose. In one aspect, the level of PCSK9 is reduced up to about 80% after administration of the dose. In one aspect, the level of PCSK9 is reduced up to about 90% after administration of the dose.

In one aspect, the level of PCSK9 in the subject is reduced about 10% to about 95% after administration of the dose. In one aspect, the level of PCSK9 in the subject is reduced about 20% to about 80% after administration of the dose. In one aspect, the level of PCSK9 in the subject is reduced about 30% to about 70% after administration of the dose. In one aspect, the level of PCSK9 in the subject is reduced about 40% to about 70% after administration of the dose. In one aspect, the level of PCSK9 in the subject is reduced about 50% to about 70% after administration of the dose.

In one aspect, the reduced PCSK9 level in the subject increases less than about 10%, for at least about 2 weeks after administering the dose. In one aspect, the reduced PCSK9 level in the subject increases less than about 5%, for at least about 2 weeks after administering the dose. In one aspect, the reduced PCSK9 level in the subject increases less than about 2%, for at least about 2 weeks after administering the dose.

In one aspect, the reduced PCSK9 level in the subject increases less than about 10%, for at least about 3 weeks after administering the dose. In one aspect, the reduced PCSK9 level in the subject increases less than about 5%, for at least about 3 weeks after administering the dose. In one aspect, the reduced PCSK9 level in the subject increases less than about 2%, for at least about 3 weeks after administering the dose.

In one aspect, the reduced PCSK9 level in the subject increases less than about 10%, for at least about 4 weeks after administering the dose. In one aspect, the reduced PCSK9 level in the subject increases less than about 5%, for at least about 4 weeks after administering the dose. In one aspect, the reduced PCSK9 level in the subject increases less than about 2%, for at least about 4 weeks after administering the dose.

In one aspect, the reduced PCSK9 level in the subject increases less than about 10%, for at least about 5 weeks after administering the dose. In one aspect, the reduced PCSK9 level in the subject increases less than about 5%, for at least about 5 weeks after administering the dose. In one aspect, the reduced PCSK9 level in the subject increases less than about 2%, for at least about 5 weeks after administering the dose.

In one aspect, the reduced PCSK9 level in the subject increases less than about 10%, for up to about 6 weeks after administering the dose. In one aspect, the reduced PCSK9 level in the subject increases less than about 5%, for up to about 6 weeks after administering the dose. In one aspect, the reduced PCSK9 level in the subject increases less than about 2%, for up to about 6 weeks after administering the dose.

In one aspect, the reduced PCSK9 level in the subject increases less than about 10%, for up to about 9 weeks after administering the dose. In one aspect, the reduced PCSK9 level in the subject increases less than about 5%, for up to about 9 weeks after administering the dose. In one aspect, the reduced PCSK9 level in the subject increases less than about 2%, for up to about 9 weeks after administering the dose.

In one aspect, the reduced PCSK9 level in the subject increases less than about 10%, for up to about 12 weeks after administering the dose. In one aspect, the reduced PCSK9 level in the subject increases less than about 5%, for up to about 12 weeks after administering the dose. In one aspect, the reduced PCSK9 level in the subject increases less than about 2%, for up to about 12 weeks after administering the dose.

Dosing Regimens with an Initial Loading Dose

In one aspect, the dosing regimen includes administering a loading dose and one or more maintenance doses of a PCSK9 inhibitor to a subject. In one aspect, the dosing regimen includes administering a loading dose and one or more maintenance doses of a PCSK9 antisense oligonucleotide to a subject. In one aspect, the dosing regimen includes administering to the subject a loading dose that includes a unit dosage of a PCSK9 antisense oligonucleotide. In one aspect, the dosing regimen includes administering one or more maintenance doses that include a unit dosage of a PCSK9 antisense oligonucleotide. In one aspect, the loading dose includes a greater amount of a PCSK9 antisense oligonucleotide than a maintenance dose. In one aspect, the loading dose includes a same amount of a PCSK9 antisense oligonucleotide as a maintenance dose.

In one aspect, the unit dosages for each of the maintenance doses are the same. In one aspect, the unit dosages for the maintenance doses are not all the same. In one aspect, the unit dosages for the maintenance doses increase over time. In one aspect, the unit dosages for the maintenance doses decrease over time.

In one aspect, the loading dose, maintenance dose or both are administered by parenteral administration. In one aspect, parenteral administration includes subcutaneous, intravenous, or intramuscular administration. In one aspect, parenteral administration includes subcutaneous administration.

In one aspect, the PCSK9 antisense oligonucleotide is administered as a “fixed dose”, e.g., in which the same dosage amount is used for all subjects regardless of subject-related factors such as weight. In one aspect, the PCSK9 antisense oligonucleotide is administered as a “weight-based” dose, e.g., in which the dosage amount can change depending on the weight of the subject.

In one aspect, the loading dose is administered at the start of treatment. In one aspect, the loading dose reduces a level of PCSK9 as compared to a level of PCSK9 before administration of the loading dose. In one aspect, the loading dose reduces a level of PCSK9 expression as compared to a level of PCSK9 expression prior to administration of the loading dose. In one aspect, the loading dose reduces a level of circulating PCSK9 as compared to a level of circulating PCSK9 expression prior to administration of the loading dose. In one aspect, the loading dose reduces a serum or plasma level of PCSK9 in the subject.

In one aspect, the loading dose reduces a LDL-C level as compared to the LDL-C level prior to administration of the loading dose. In one aspect, the loading dose reduces a circulating level of LDL-C as compared to the LDL-C level prior to administration of the loading dose. In one aspect, the loading dose reduces a plasma level of LDL-C. In one aspect, the loading dose reduces a serum level of LDL-C.

In one aspect, one or more maintenance doses are administered to the subject during a maintenance period. The number of maintenance doses administered during the maintenance period, or the duration of the maintenance period can vary depend on the desired outcome, for example, reducing a level of PCSK9 in a subject, reducing expression of PCSK9 in a subject, reducing a level of LDL-C in a subject, or reducing one or more symptoms of a disease associated with PCSK9 in a subject. In one aspect, the maintenance period includes about 1, about 2, about 3, about 4, about 5, about 6, about 7, about 8, about 9, about 10, about 11, about 12, about 13, about 14, about 15, about 16, about 17, about 18, about 19, or about 20 or more maintenance doses. In one aspect, the duration of the maintenance period is about 3 months, about 6 months, about 9 months, about 12 months, about 15 months, about 24 months or about 2 years, about 3 years, about 4 years, about 5 years, about 6 years, about 7 years, about 8 years, about 9 years, about 10 years or more, e.g., chronic administration. In one aspect, the maintenance period is the lifetime of the subject. In one aspect, the maintenance period includes more than one dose and the unit dosages administered during the maintenance period are all the same. In one aspect, the unit dosages administered during the maintenance period are not all the same. In one aspect, the amount of the PCSK9 inhibitor in the unit dosages administered during the maintenance period increase over time. In one aspect, the amount of the PCSK9 inhibitor in the unit dosages administered during the maintenance period decrease over time.

In one aspect, the maintenance dose is administered to the subject about every 2 weeks to about every 6 weeks. In one aspect, the maintenance dose is administered to the subject at an interval of about once a week. In one aspect, the maintenance dose is administered to the subject at an interval of about once every two weeks. In one aspect, the maintenance dose is administered to the subject at an interval of about once every three weeks. In one aspect, the maintenance dose is administered to the subject at an interval of about once every 4 weeks. In one aspect, the maintenance dose is administered to the subject at an interval of about once every 5 weeks. In one aspect, the maintenance dose is administered to the subject at an interval of about once every 6 weeks.

In one aspect, the maintenance dose is administered to the subject about every 2 weeks for at least about 6 weeks. In one aspect, the maintenance dose is administered to the subject about every 2 weeks for at least about 12 weeks. In one aspect, the maintenance dose is administered to the subject about every 2 weeks for at least about 26 weeks. In one aspect, the maintenance dose is administered to the subject about every 2 weeks for at least about 52 weeks. In one aspect, the maintenance dose is administered to the subject about every 3 weeks for at least about 12 weeks. In one aspect, the maintenance dose is administered to the subject about every 3 weeks for at least about 26 weeks. In one aspect, the maintenance dose is administered to the subject about every 3 weeks for at least about 52 weeks. In one aspect, the maintenance dose is administered to the subject about every 4 weeks for at least about 12 weeks. In one aspect, the maintenance dose is administered to the subject about every 4 weeks for at least about 26 weeks. In one aspect, the maintenance dose is administered to the subject about every 4 weeks for at least about 52 weeks. In one aspect, the maintenance dose is administered to the subject about every 5 weeks for at least about 12 weeks. In one aspect, the maintenance dose is administered to the subject about every 5 weeks for at least about 26 weeks. In one aspect, the maintenance dose is administered to the subject about every 5 weeks for at least about 52 weeks. In one aspect, the maintenance dose is administered to the subject about every 6 weeks for at least about 12 weeks. In one aspect, the maintenance dose is administered to the subject about every 6 weeks for at least about 26 weeks. In one aspect, the maintenance dose is administered to the subject about every 6 weeks for at least about 52 weeks.

In one aspect, administration of a maintenance dose is repeated monthly. In one aspect, the maintenance dose is administered to the subject once a month for at least about 3 months. In one aspect, the maintenance dose is administered to the subject once a month for at least about 6 months. In one aspect, the maintenance dose is administered to the subject once a month for at least about 9 months. In one aspect, the maintenance dose is administered to the subject once a month for at least about 12 months. In one aspect, the maintenance dose is administered to the subject once a month for at least about 15 months. In one aspect, the maintenance dose is administered to the subject once a month for at least about 24 months.

In one aspect, administration of a maintenance dose is repeated once about every 21 days. In one aspect, administration of a maintenance dose is repeated once about every 28 days. In one aspect, administration of a maintenance dose is repeated once about every 30 days.

In one aspect, the maintenance dose is administered to the subject about every 21 days for at least about 90 days. In one aspect, the maintenance dose is administered to the subject about every 21 days for at least about 120 days. In one aspect, the maintenance dose is administered to the subject about every 21 days for at least about 180 days. In one aspect, the maintenance dose is administered to the subject about every 21 days for at least about 365 days.

In one aspect, the maintenance dose is administered to the subject about every 28 days for at least about 90 days. In one aspect, the maintenance dose is administered to the subject about every 28 days for at least about 120 days. In one aspect, the maintenance dose is administered to the subject about every 28 days for at least about 180 days. In one aspect, the maintenance dose is administered to the subject about every 28 days for at least about 365 days.

In one aspect, the maintenance dose is administered to the subject about every 30 days for at least about 90 days. In one aspect, the maintenance dose is administered to the subject about every 30 days for at least about 120 days. In one aspect, the maintenance dose is administered to the subject about every 30 days for at least about 180 days. In one aspect, the maintenance dose is administered to the subject about every 30 days for at least about 365 days.

In one aspect, the dosing regimen includes administering the PCKS9 antisense oligonucleotide as a unit dosage of at least about 10 mg, or at least about 20 mg, and up to about 150 mg. In one aspect, the dosing regimen includes administering the PCKS9 antisense oligonucleotide as a unit dosage of at least about 10 mg, or at least about 20 mg, and up to about 120 mg. In one aspect, the dosing regimen includes administering the PCKS9 antisense oligonucleotide as a unit dosage of at least about 12 mg. In one aspect, the dosing regimen includes administering the PCKS9 antisense oligonucleotide as a unit dosage of at least about 10 mg. In one aspect, the dosing regimen includes administering the PCKS9 antisense oligonucleotide as a unit dosage of at least about 15 mg. In one aspect, the dosing regimen includes administering the PCKS9 antisense oligonucleotide as a unit dosage of at least about 20 mg. In one aspect, the dosing regimen includes administering the PCKS9 antisense oligonucleotide as a unit dosage of at least about 25 mg. In one aspect, the dosing regimen includes administering the PCKS9 antisense oligonucleotide as a unit dosage of at least about 30 mg. In one aspect, the dosing regimen includes administering the PCKS9 antisense oligonucleotide as a unit dosage of at least about 40 mg. In one aspect, the dosing regimen includes administering the PCKS9 antisense oligonucleotide as a unit dosage of at least about 50 mg. In one aspect, the dosing regimen includes administering the PCKS9 antisense oligonucleotide as a unit dosage of at least about 60 mg. In one aspect, the dosing regimen includes administering the PCKS9 antisense oligonucleotide as a unit dosage of at least about 70 mg. In one aspect, the dosing regimen includes administering the PCKS9 antisense oligonucleotide as a unit dosage of at least about 80 mg. In one aspect, the dosing regimen includes administering the PCKS9 antisense oligonucleotide as a unit dosage of at least about 90 mg. In one aspect, the dosing regimen includes administering the PCKS9 antisense oligonucleotide as a unit dosage of up to about 100 mg. In one aspect, the dosing regimen includes administering the PCKS9 antisense oligonucleotide as a unit dosage of up to about 110 mg. In one aspect, the dosing regimen includes administering the PCKS9 antisense oligonucleotide as a unit dosage of up to about 120 mg. In one aspect, the dosing regimen includes administering the PCKS9 antisense oligonucleotide as a unit dosage of up to about 130 mg. In one aspect, the dosing regimen includes administering the PCKS9 antisense oligonucleotide as a unit dosage of up to about 140 mg. In one aspect, the dosing regimen includes administering the PCKS9 antisense oligonucleotide as a unit dosage of up to about 150 mg.

In one aspect, the dosing regimen includes administering the PCSK9 antisense oligonucleotide as a unit dosage of about 10 mg to about 150 mg. In one aspect, the dosing regimen includes administering the PCSK9 antisense oligonucleotide as a unit dosage of about 20 mg to about 120 mg. In one aspect, the dosing regimen includes administering the PCSK9 antisense oligonucleotide as a unit dosage of about 50 mg to about 100 mg. In one aspect, the dosing regimen includes administering the PCSK9 antisense oligonucleotide as a unit dosage of about 50 mg to about 90 mg. In one aspect, the dosing regimen includes administering the PCSK9 antisense oligonucleotide as a unit dosage of about 50 mg to about 60 mg. In one aspect, the dosing regimen includes administering the PCSK9 antisense oligonucleotide as a unit dosage of about 60 mg to about 70 mg. In one aspect, the dosing regimen includes administering the PCSK9 antisense oligonucleotide as a unit dosage of about 70 mg to about 80 mg. In one aspect, the dosing regimen includes administering the PCSK9 antisense oligonucleotide as a unit dosage of about 80 mg to about 90 mg. In one aspect, the dosing regimen includes administering the PCSK9 antisense oligonucleotide as a unit dosage of about 90 mg to about 100 mg.

In one aspect, the dosing regimen includes administering the PCSK9 antisense oligonucleotide as a unit dosage of about 10 mg. In one aspect, the dosing regimen includes administering the PCSK9 antisense oligonucleotide as a unit dosage of about 15 mg. In one aspect, the dosing regimen includes administering the PCSK9 antisense oligonucleotide as a unit dosage of about 20 mg. In one aspect, the dosing regimen includes administering the PCSK9 antisense oligonucleotide as a unit dosage of about 30 mg. In one aspect, the dosing regimen includes administering the PCSK9 antisense oligonucleotide as a unit dosage of about 40 mg. In one aspect, the dosing regimen includes administering the PCSK9 antisense oligonucleotide as a unit dosage of PCSK9 antisense oligonucleotide of about 50 mg. In one aspect, the dosing regimen includes administering the PCSK9 antisense oligonucleotide as a unit dosage of PCSK9 antisense oligonucleotide of about 60 mg. In one aspect, the dosing regimen includes administering the PCSK9 antisense oligonucleotide as a unit dosage of PCSK9 antisense oligonucleotide of about 70 mg. In one aspect, the dosing regimen includes administering the PCSK9 antisense oligonucleotide as a unit dosage of PCSK9 antisense oligonucleotide of about 80 mg. In one aspect, the dosing regimen includes administering the PCSK9 antisense oligonucleotide as a unit dosage of PCSK9 antisense oligonucleotide of about 90 mg. In one aspect, the dosing regimen includes administering the PCSK9 antisense oligonucleotide as a unit dosage of PCSK9 antisense oligonucleotide of about 100 mg. In one aspect, the dosing regimen includes administering the PCSK9 antisense oligonucleotide as a unit dosage of PCSK9 antisense oligonucleotide of about 110 mg. In one aspect, the dosing regimen includes administering the PCSK9 antisense oligonucleotide as a unit dosage of PCSK9 antisense oligonucleotide of about 120 mg. In one aspect, the dosing regimen includes administering the PCSK9 antisense oligonucleotide as a unit dosage of PCSK9 antisense oligonucleotide of about 130 mg. In one aspect, the dosing regimen includes administering the PCSK9 antisense oligonucleotide as a unit dosage of PCSK9 antisense oligonucleotide of about 140 mg. In one aspect, the dosing regimen includes administering the PCSK9 antisense oligonucleotide as a unit dosage of PCSK9 antisense oligonucleotide of about 150 mg. In one aspect, the antisense oligonucleotide is administered in a unit dosage of about 50 mg, about 60 mg, about 70 mg, about 80 mg, or about 90 mg.

In one aspect, the dosing regimen includes administering a loading dose to the subject, wherein the loading dose includes a unit dosage of a PCSK9 antisense oligonucleotide. In one aspect, the dosing regimen further includes administering one or more maintenance doses to the subject, wherein each maintenance dose includes a unit dosage or a PCSK9 antisense oligonucleotide. In one aspect, the loading dose includes a greater amount of a PCSK9 antisense oligonucleotide or a pharmaceutically acceptable salt thereof than the maintenance dose. In one aspect, the loading dose includes a same amount of a PCSK9 antisense oligonucleotide or a pharmaceutically acceptable salt thereof as the maintenance dose. In one aspect, each of the maintenance doses include the same unit dosage amount. In one aspect, one or more maintenance doses do not include the same unit dosage amount. In one aspect, the unit dosage of the maintenance doses increases over time. In one aspect, the unit dosages of the maintenance doses decrease over time.

In one aspect, the loading dose, maintenance dose or both are administered by parenteral administration. In one aspect, parenteral administration includes subcutaneous, intravenous, or intramuscular administration. In one aspect, parenteral administration includes subcutaneous administration.

In one aspect, a loading dose of the PCSK9 antisense oligonucleotide is administered in a unit dosage of at least about 10 mg. In one aspect, a loading dose of the PCSK9 antisense oligonucleotide is administered in a unit dosage of at least about 12 mg. In one aspect, a loading dose of the PCSK9 antisense oligonucleotide is administered in a unit dosage of at least about 15 mg. In one aspect, a loading dose of the PCSK9 antisense oligonucleotide is administered in a unit dosage of at least about 20 mg. In one aspect, a loading dose of the PCSK9 antisense oligonucleotide is administered in a unit dosage of at least about 25 mg. In one aspect, a loading dose of the PCSK9 antisense oligonucleotide is administered in a unit dosage of at least about 30 mg. In one aspect, a loading dose of the PCSK9 antisense oligonucleotide is administered in a unit dosage of at least about 40 mg. In one aspect, a loading dose of the PCSK9 antisense oligonucleotide is administered in a unit dosage of at least about 50 mg. In one aspect, a loading dose of the PCSK9 antisense oligonucleotide is administered in a unit dosage of at least about 60 mg. In one aspect, a loading dose of the PCSK9 antisense oligonucleotide is administered in a unit dosage of at least about 70 mg. In one aspect, a loading dose of the PCSK9 antisense oligonucleotide is administered in a unit dosage of at least about 80 mg. In one aspect, a loading dose of the PCSK9 antisense oligonucleotide is administered in a unit dosage of at least about 90 mg. In one aspect, a loading dose of the PCSK9 antisense oligonucleotide is administered in a unit dosage of up to about 100 mg. In one aspect, a loading dose of the PCSK9 antisense oligonucleotide is administered in a unit dosage of up to and up to about 110 mg. In one aspect, a loading dose of the PCSK9 antisense oligonucleotide is administered in a unit dosage of up to about 120 mg. In one aspect, a loading dose of the PCSK9 antisense oligonucleotide is administered in a unit dosage of up to about 130 mg. In one aspect, a loading dose of the PCSK9 antisense oligonucleotide is administered in a unit dosage of up to about 140 mg. In one aspect, a loading dose of the PCSK9 antisense oligonucleotide is administered in a unit dosage of up to about 150 mg.

In one aspect, a loading dose of the PCSK9 antisense oligonucleotide is administered in a unit dosage of about 10 mg, or at least about 20 mg, to about 150 mg. In one aspect, a loading dose of the PCSK9 antisense oligonucleotide is administered in a unit dosage of about 20 mg to about 120 mg. In one aspect, a loading dose of the PCSK9 antisense oligonucleotide is administered in a unit dosage of about 50 mg to about 100 mg. In one aspect, a loading dose of the PCSK9 antisense oligonucleotide is administered in a unit dosage of about 50 mg to about 90 mg. In one aspect, a loading dose of the PCSK9 antisense oligonucleotide is administered in a unit dosage of about 50 mg to about 60 mg. In one aspect, a loading dose of the PCSK9 antisense oligonucleotide is administered in a unit dosage of about 60 mg to about 70 mg. In one aspect, a loading dose of the PCSK9 antisense oligonucleotide is administered in a unit dosage of about 70 mg to about 80 mg. In one aspect, a loading dose of the PCSK9 antisense oligonucleotide is administered in a unit dosage of about 80 mg to about 90 mg. In one aspect, a loading dose of the PCSK9 antisense oligonucleotide is administered in a unit dosage of about 90 mg to about 100 mg.

In one aspect, a loading dose of the PCSK9 antisense oligonucleotide is administered in a unit dosage of about 10 mg. In one aspect, a loading dose of the PCSK9 antisense oligonucleotide is administered in a unit dosage of about 15 mg. In one aspect, a loading dose of the PCSK9 antisense oligonucleotide is administered in a unit dosage of about 20 mg. In one aspect, a loading dose of the PCSK9 antisense oligonucleotide is administered in a unit dosage of about 30 mg. In one aspect, a loading dose of the PCSK9 antisense oligonucleotide is administered in a unit dosage of about 40 mg. In one aspect, a loading dose of the PCSK9 antisense oligonucleotide is administered in a unit dosage of about 50 mg. In one aspect, a loading dose of the PCSK9 antisense oligonucleotide is administered in a unit dosage of about 60 mg. In one aspect, a loading dose of the PCSK9 antisense oligonucleotide is administered in a unit dosage of about 70 mg. In one aspect, a loading dose of the PCSK9 antisense oligonucleotide is administered in a unit dosage of about 80 mg. In one aspect, a loading dose of the PCSK9 antisense oligonucleotide is administered in a unit dosage of about 90 mg. In one aspect, a loading dose of the PCSK9 antisense oligonucleotide is administered in a unit dosage of about 100 mg. In one aspect, a loading dose of the PCSK9 antisense oligonucleotide is administered in a unit dosage of about 110 mg. In one aspect, a loading dose of the PCSK9 antisense oligonucleotide is administered in a unit dosage of about 120 mg. In one aspect, a loading dose of the PCSK9 antisense oligonucleotide is administered in a unit dosage of about 130 mg. In one aspect, a loading dose of the PCSK9 antisense oligonucleotide is administered in a unit dosage of about 140 mg. In one aspect, a loading dose of the PCSK9 antisense oligonucleotide is administered in a unit dosage of about or about 150 mg. In one aspect, a loading dose of the PCSK9 antisense oligonucleotide is administered in a unit dosage of about 50 mg, about 60 mg, about 70 mg, about 80 mg, or about 90 mg.

In one aspect, a maintenance dose of the PCSK9 antisense oligonucleotide is administered in a unit dosage of at least about 10 mg. In one aspect, a maintenance dose of the PCSK9 antisense oligonucleotide is administered in a unit dosage of at least about 12 mg. In one aspect, a maintenance dose of the PCSK9 antisense oligonucleotide is administered in a unit dosage of at least about 15 mg. In one aspect, a maintenance dose of the PCSK9 antisense oligonucleotide is administered in a unit dosage of at least about 20 mg. In one aspect, a maintenance dose of the PCSK9 antisense oligonucleotide is administered in a unit dosage of at least about 25 mg. In one aspect, a maintenance dose of the PCSK9 antisense oligonucleotide is administered in a unit dosage of at least about 30 mg. In one aspect, a maintenance dose of the PCSK9 antisense oligonucleotide is administered in a unit dosage of at least about 40 mg. In one aspect, a maintenance dose of the PCSK9 antisense oligonucleotide is administered in a unit dosage of at least about 50 mg. In one aspect, a maintenance dose of the PCSK9 antisense oligonucleotide is administered in a unit dosage of at least about 60 mg. In one aspect, a maintenance dose of the PCSK9 antisense oligonucleotide is administered in a unit dosage of at least about 70 mg. In one aspect, a maintenance dose of the PCSK9 antisense oligonucleotide is administered in a unit dosage of at least about 80 mg. In one aspect, a maintenance dose of the PCSK9 antisense oligonucleotide is administered in a unit dosage of at least about 90 mg. In one aspect, a maintenance dose of the PCSK9 antisense oligonucleotide is administered in a unit dosage of up to about 100 mg. In one aspect, a maintenance dose of the PCSK9 antisense oligonucleotide is administered in a unit dosage of up to about 110 mg. In one aspect, a maintenance dose of the PCSK9 antisense oligonucleotide is administered in a unit dosage of up to about 120 mg. In one aspect, a maintenance dose of the PCSK9 antisense oligonucleotide is administered in a unit dosage of up to about 130 mg. In one aspect, a maintenance dose of the PCSK9 antisense oligonucleotide is administered in a unit dosage of up to about 140 mg. In one aspect, a maintenance dose of the PCSK9 antisense oligonucleotide is administered in a unit dosage of about 150 mg.

In one aspect, a maintenance dose of the PCSK9 antisense oligonucleotide is administered in a unit dosage of about 10 mg, or at least about 20 mg, to about 150 mg, In one aspect, a maintenance dose of the PCSK9 antisense oligonucleotide is administered in a unit dosage of about 20 mg to about 120 mg. In one aspect, a maintenance dose of the PCSK9 antisense oligonucleotide is administered in a unit dosage of about 50 mg to about 100 mg. In one aspect, a maintenance dose of the PCSK9 antisense oligonucleotide is administered in a unit dosage of about 50 mg to about 90 mg. In one aspect, a maintenance dose of the PCSK9 antisense oligonucleotide is administered in a unit dosage of about 50 mg to about 60 mg. In one aspect, a maintenance dose of the PCSK9 antisense oligonucleotide is administered in a unit dosage of about 60 mg to about 70 mg. In one aspect, a maintenance dose of the PCSK9 antisense oligonucleotide is administered in a unit dosage of about 70 mg to about 80 mg. In one aspect, a maintenance dose of the PCSK9 antisense oligonucleotide is administered in a unit dosage of about 80 mg to about 90 mg. In one aspect, a maintenance dose of the PCSK9 antisense oligonucleotide is administered in a unit dosage of about 90 mg to about 100 mg.

In one aspect, a maintenance dose of the PCSK9 antisense oligonucleotide is administered in a unit dosage of about 10 mg. In one aspect, a maintenance dose of the PCSK9 antisense oligonucleotide is administered in a unit dosage of about 12 mg. In one aspect, a maintenance dose of the PCSK9 antisense oligonucleotide is administered in a unit dosage of about 15 mg. In one aspect, a maintenance dose of the PCSK9 antisense oligonucleotide is administered in a unit dosage of about 20 mg. In one aspect, a maintenance dose of the PCSK9 antisense oligonucleotide is administered in a unit dosage of about 25 mg. In one aspect, a maintenance dose of the PCSK9 antisense oligonucleotide is administered in a unit dosage of about 30 mg. In one aspect, a maintenance dose of the PCSK9 antisense oligonucleotide is administered in a unit dosage of about 40 mg. In one aspect, a maintenance dose of the PCSK9 antisense oligonucleotide is administered in a unit dosage of about 50 mg. In one aspect, a maintenance dose of the PCSK9 antisense oligonucleotide is administered in a unit dosage of about 60 mg. In one aspect, a maintenance dose of the PCSK9 antisense oligonucleotide is administered in a unit dosage of about 70 mg. In one aspect, a maintenance dose of the PCSK9 antisense oligonucleotide is administered in a unit dosage of about 80 mg. In one aspect, a maintenance dose of the PCSK9 antisense oligonucleotide is administered in a unit dosage of about 90 mg. In one aspect, a maintenance dose of the PCSK9 antisense oligonucleotide is administered in a unit dosage of about 100 mg. In one aspect, a maintenance dose of the PCSK9 antisense oligonucleotide is administered in a unit dosage of about 110 mg. In one aspect, a maintenance dose of the PCSK9 antisense oligonucleotide is administered in a unit dosage of about 120 mg. In one aspect, a maintenance dose of the PCSK9 antisense oligonucleotide is administered in a unit dosage of about 130 mg. In one aspect, a maintenance dose of the PCSK9 antisense oligonucleotide is administered in a unit dosage of about 140 mg. In one aspect, a maintenance dose of the PCSK9 antisense oligonucleotide is administered in a unit dosage of about or about 150 mg. In one aspect, a maintenance dose of the PCSK9 antisense oligonucleotide is administered in a unit dosage of about 50 mg, about 60 mg, about 70 mg, about 80 mg, or about 90 mg.

In one aspect, the dosing regimen includes administering one or more, or a plurality of maintenance doses to the subject about every 2 weeks to about every 6 weeks. In one aspect, a maintenance dose is administered to the subject about every 2 weeks, about every 3 weeks, about every 4 weeks, or about every 5 weeks and up to about every 6 weeks, for at least about 6 weeks, at least about 12 weeks, at least about 26 weeks, or at least about 52 weeks. In one aspect, a maintenance dose is administered to the subject once a month for at least about 3 months. In one aspect, a maintenance dose is administered to the subject about once a month for at least about 6 months, at least about 9 months, at least about 12 months, at least about 15 months or at least about 24 months. In one aspect, a maintenance dose is administered to the subject about every 21 days, about every 28 days or about every 30 days for at least about 90 days, at least about 120 days, at least about 180 days, or at least about 365 days.

In one aspect, the LDL-C level in the subject is reduced about 10% to about 95% after administering the loading dose to provide a reduced LDL-C level in the subject. In one aspect, the level of LDL-C in the subject is reduced at least about 20% after administering the loading dose. In one aspect, the level of LDL-C in the subject is reduced at least about 30% after administering the loading dose. In one aspect, the level of LDL-C in the subject is reduced at least about 40% after administering the loading dose. In one aspect, the level of LDL-C in the subject is reduced at least about 50% after administering the loading dose. In one aspect, the level of LDL-C in the subject is reduced at least about 60% after administering the loading dose. In one aspect, the level of LDL-C in the subject is reduced at least about 70% after administering the loading dose. In one aspect, the level of LDL-C in the subject is reduced up to about 80% after administering the loading dose. In one aspect, the level of LDL-C in the subject is reduced up to about 90% after administering the loading dose.

In one aspect, the level of LDL-C in the subject is reduced about 20% to about 80% after administering the loading dose. In one aspect, the level of LDL-C in the subject is reduced about 30% to about 70% after administering the loading dose. In one aspect, the level of LDL-C in the subject is reduced about 40% to about 70% after administering the loading dose. In one aspect, the level of LDL-C in the subject is reduced about 50% to about 70% after administering the loading dose.

In one aspect, the reduced LDL-C level in the subject increases less than about 10% for at least about 2 weeks after administering the loading dose. In one aspect, the reduced LDL-C level in the subject increases less than about 5% for at least about 2 weeks after administering the loading dose. In one aspect, the LDL-C level in the subject increases less than about 2% for at least about 2 weeks after administering the loading dose.

In one aspect, the reduced LDL-C level in the subject increases less than about 10% for at least about 3 weeks after administering the loading dose. In one aspect, the reduced LDL-C level in the subject increases less than about 5% for at least about 3 weeks after administering the loading dose. In one aspect, the LDL-C level in the subject increases less than about 2% for at least about 3 weeks after administering the loading dose.

In one aspect, the reduced LDL-C level in the subject increases less than about 10% for at least about 4 weeks after administering the loading dose. In one aspect, the reduced LDL-C level in the subject increases less than about 5% for at least about 4 weeks after administering the loading dose. In one aspect, the LDL-C level in the subject increases less than about 2% for at least about 4 weeks after administering the loading dose.

In one aspect, the reduced LDL-C level in the subject increases less than about 10% for at least about 5 weeks after administering the loading dose. In one aspect, the reduced LDL-C level in the subject increases less than about 5% for at least about 5 weeks after administering the loading dose. In one aspect, the LDL-C level in the subject increases less than about 2% for at least about 5 weeks after administering the loading dose.

In one aspect, the reduced LDL-C level in the subject increases less than about 10% for up to about 6 weeks after administering the loading dose. In one aspect, the reduced LDL-C level in the subject increases less than about 5% for up to about 6 weeks after administering the loading dose. In one aspect, the LDL-C level in the subject increases less than about 2% for up to about 6 weeks after administering the loading dose.

In one aspect, the reduced LDL-C level in the subject increases less than about 10% for up to about 9 weeks after administering the loading dose. In one aspect, the reduced LDL-C level in the subject increases less than about 5% for up to about 9 weeks after administering the loading dose. In one aspect, the LDL-C level in the subject increases less than about 2% for up to about 9 weeks after administering the loading dose.

In one aspect, the reduced LDL-C level in the subject increases less than about 10% for up to about 12 weeks after administering the loading dose. In one aspect, the reduced LDL-C level in the subject increases less than about 5% for up to about 12 weeks after administering the loading dose. In one aspect, the LDL-C level in the subject increases less than about 2% for up to about 12 weeks after administering the loading dose.

In one aspect, the reduced LDL-C level in the subject increases less than about 10% for at least about 2 weeks after administering the maintenance dose. In one aspect, the reduced LDL-C level in the subject increases less than about 5% for at least about 2 weeks after administering the maintenance dose. In one aspect, the reduced LDL-C level in the subject increases less than about 2% for at least about 2 weeks after administering the maintenance dose.

In one aspect, the reduced LDL-C level in the subject increases less than about 10% for at least about 3 weeks after administering the maintenance dose. In one aspect, the reduced LDL-C level in the subject increases less than about 5% for at least about 3 weeks after administering the maintenance dose. In one aspect, the reduced LDL-C level in the subject increases less than about 2% for at least about 3 weeks after administering the maintenance dose.

In one aspect, the reduced LDL-C level in the subject increases less than about 10% for at least about 4 weeks after administering the maintenance dose. In one aspect, the reduced LDL-C level in the subject increases less than about 5% for at least about 4 weeks after administering the maintenance dose. In one aspect, the reduced LDL-C level in the subject increases less than about 2% for at least about 4 weeks after administering the maintenance dose.

In one aspect, the reduced LDL-C level in the subject increases less than about 10% for at least about 5 weeks after administering the maintenance dose. In one aspect, the reduced LDL-C level in the subject increases less than about 5% for at least about 5 weeks after administering the maintenance dose. In one aspect, the reduced LDL-C level in the subject increases less than about 2% for at least about 5 weeks after administering the maintenance dose.

In one aspect, the reduced LDL-C level in the subject increases less than about 10% for up to about 6 weeks after administering the maintenance dose. In one aspect, the reduced LDL-C level in the subject increases less than about 5% for up to about 6 weeks after administering the maintenance dose. In one aspect, the reduced LDL-C level in the subject increases less than about 2% for up to about 6 weeks after administering the maintenance dose.

In one aspect, the reduced LDL-C level in the subject increases less than about 10% for up to about 9 weeks after administering the maintenance dose. In one aspect, the reduced LDL-C level in the subject increases less than about 5% for up to about 9 weeks after administering the maintenance dose. In one aspect, the reduced LDL-C level in the subject increases less than about 2% for up to about 9 weeks after administering the maintenance dose.

In one aspect, the reduced LDL-C level in the subject increases less than about 10% for up to about 12 weeks after administering the maintenance dose. In one aspect, the reduced LDL-C level in the subject increases less than about 5% for up to about 12 weeks after administering the maintenance dose. In one aspect, the reduced LDL-C level in the subject increases less than about 2% for up to about 12 weeks after administering the maintenance dose.

In one level, a circulating level of LDL-Cis reduced. In one aspect, a serum level of LCL-C is reduced. In one aspect, a plasma level of LDL-C in the subject is reduced.

In one aspect, the subject has a serum low-density lipoprotein cholesterol (LDL-C) level of at least about 100 mg/dL prior to administration of the loading dose. In one aspect, the subject has a serum low-density lipoprotein cholesterol (LDL-C) level of at least about 110 mg/dL prior to administration of the loading dose. In one aspect, the subject has a serum low-density lipoprotein cholesterol (LDL-C) level of at least about 120 mg/dL prior to administration of the loading dose. In one aspect, the subject has a serum low-density lipoprotein cholesterol (LDL-C) level of at least about 130 mg/dL prior to administration of the loading dose. In one aspect, the subject has a serum low-density lipoprotein cholesterol (LDL-C) level of at least about 140 mg/dL prior to administration of the loading dose. In one aspect, the subject has a serum low-density lipoprotein cholesterol (LDL-C) level of at least about 150 mg/dL prior to administration of the loading dose. In one aspect, the subject has a serum low-density lipoprotein cholesterol (LDL-C) level of at least about 160 mg/dL prior to administration of the loading dose. In one aspect, the subject has a serum low-density lipoprotein cholesterol (LDL-C) level of at least about 170 mg/dL prior to administration of the loading dose. In one aspect, the subject has a serum low-density lipoprotein cholesterol (LDL-C) level of at least about 180 mg/dL prior to administration of the loading dose. In one aspect, the subject has a serum low-density lipoprotein cholesterol (LDL-C) level of at least about 190 mg/dL prior to administration of the loading dose. In one aspect, the subject has a serum low-density lipoprotein cholesterol (LDL-C) level of at least about 200 mg/dL prior to administration of the loading dose. In one aspect, the subject has a serum low-density lipoprotein cholesterol (LDL-C) level of at least about 300 mg/dL prior to administration of the loading dose. In one aspect, the subject has a serum low-density lipoprotein cholesterol (LDL-C) level of at least about 400 mg/dL prior to administration of the loading dose.

In one aspect, the subject has a serum low-density lipoprotein cholesterol (LDL-C) level of less than about 70 mg/dL for at least about 2 weeks and up to about 6 weeks after administration of the loading dose. In one aspect, the subject has a serum low-density lipoprotein cholesterol (LDL-C) level of less than about 80 mg/dL for at least about 2 weeks and up to about 6 weeks after administration of the loading dose. In one aspect, the subject has a serum low-density lipoprotein cholesterol (LDL-C) level of less than about 90 mg/dL for at least about 2 weeks and up to about 6 weeks after administration of the loading dose. In one aspect, the subject has a serum low-density lipoprotein cholesterol (LDL-C) level of less than about 100 mg/dL for at least about 2 weeks and up to about 6 weeks after administration of the loading dose. In one aspect, the subject has a serum low-density lipoprotein cholesterol (LDL-C) level of less than about 110 mg/dL for at least about 2 weeks and up to about 6 weeks after administration of the loading dose. In one aspect, the subject has a serum low-density lipoprotein cholesterol (LDL-C) level of less than about 120 mg/dL for at least about 2 weeks and up to about 6 weeks after administration of the loading dose. In one aspect, the subject has a serum low-density lipoprotein cholesterol (LDL-C) level of less than about 130 mg/dL for at least about 2 weeks and up to about 6 weeks after administration of the loading dose.

In one aspect, a dosing regimen is provided for reducing a level of PCSK9 in a subject. In one aspect, a dosing regimen is provided for reducing PCSK9 expression. In one aspect, a dosing regimen is provided for reducing a circulating level of PCSK9 in a subject. In one aspect, a dosing regimen is provided for reducing a plasma level of PCSK9 in a subject. In one aspect, a dosing regimen is provided for reducing a serum level of PCSK9 in a subject.

In one aspect, the level of PCSK9 in the subject is reduced about 10% to about 95% after administration of a PCSK9 inhibitor described herein. In one aspect, the level of PCSK9 in the subject is reduced about 10% to about 95% after administration of a PCSK9 antisense oligonucleotide described herein.

In one aspect, the level of PCSK9 is reduced at least about 10% after administration of a PCSK9 antisense oligonucleotide. In one aspect, the level of PCSK9 is reduced at least about 20% after administration of a PCSK9 antisense oligonucleotide. In one aspect, the level of PCSK9 is reduced at least about 30% after administration of a PCSK9 antisense oligonucleotide. In one aspect, the level of PCSK9 is reduced at least about 40% after administration of a PCSK9 antisense oligonucleotide. In one aspect, the level of PCSK9 is reduced at least about 50% after administration of a PCSK9 antisense oligonucleotide. In one aspect, the level of PCSK9 is reduced at least about 60% after administration of a PCSK9 antisense oligonucleotide. In one aspect, the level of PCSK9 is reduced at least about 70% after administration of a PCSK9 antisense oligonucleotide. In one aspect, the level of PCSK9 is reduced up to about 80% after administration of a PCSK9 antisense oligonucleotide. In one aspect, the level of PCSK9 is reduced up to about 90% after administration of a PCSK9 antisense oligonucleotide.

In one aspect, the level of PCSK9 in the subject is reduced about 10% to about 95% after administration of the PCSK9 antisense oligonucleotide. In one aspect, the level of PCSK9 in the subject is reduced about 20% to about 80% after administration of the PCSK9 antisense oligonucleotide. In one aspect, the level of PCSK9 in the subject is reduced about 30% to about 70% after administration of the PCSK9 antisense oligonucleotide. In one aspect, the level of PCSK9 in the subject is reduced about 40% to about 70% after administration of the PCSK9 antisense oligonucleotide. In one aspect, the level of PCSK9 in the subject is reduced about 50% to about 70% after administration of the PCSK9 antisense oligonucleotide.

In one aspect, the reduced PCSK9 level in the subject increases less than about 10%, for at least about 2 weeks after administering the PCSK9 antisense oligonucleotide. In one aspect, the reduced PCSK9 level in the subject increases less than about 5%, for at least about 2 weeks after administering the PCSK9 antisense oligonucleotide. In one aspect, the reduced PCSK9 level in the subject increases less than about 2%, for at least about 2 weeks after administering the PCSK9 antisense oligonucleotide.

In one aspect, the reduced PCSK9 level in the subject increases less than about 10%, for at least about 3 weeks after administering the PCSK9 antisense oligonucleotide. In one aspect, the reduced PCSK9 level in the subject increases less than about 5%, for at least about 3 weeks after administering the PCSK9 antisense oligonucleotide. In one aspect, the reduced PCSK9 level in the subject increases less than about 2%, for at least about 3 weeks after administering the PCSK9 antisense oligonucleotide.

In one aspect, the reduced PCSK9 level in the subject increases less than about 10%, for at least about 4 weeks after administering the PCSK9 antisense oligonucleotide. In one aspect, the reduced PCSK9 level in the subject increases less than about 5%, for at least about 4 weeks after administering the PCSK9 antisense oligonucleotide. In one aspect, the reduced PCSK9 level in the subject increases less than about 2%, for at least about 4 weeks after administering the PCSK9 antisense oligonucleotide.

In one aspect, the reduced PCSK9 level in the subject increases less than about 10%, for at least about 5 weeks after administering the PCSK9 antisense oligonucleotide. In one aspect, the reduced PCSK9 level in the subject increases less than about 5%, for at least about 5 weeks after administering the PCSK9 antisense oligonucleotide. In one aspect, the reduced PCSK9 level in the subject increases less than about 2%, for at least about 5 weeks after administering the PCSK9 antisense oligonucleotide.

In one aspect, the reduced PCSK9 level in the subject increases less than about 10%, for up to about 6 weeks after administering the PCSK9 antisense oligonucleotide. In one aspect, the reduced PCSK9 level in the subject increases less than about 5%, for up to about 6 weeks after administering the PCSK9 antisense oligonucleotide. In one aspect, the reduced PCSK9 level in the subject increases less than about 2%, for up to about 6 weeks after administering the PCSK9 antisense oligonucleotide.

In one aspect, the reduced PCSK9 level in the subject increases less than about 10%, for up to about 9 weeks after administering the PCSK9 antisense oligonucleotide. In one aspect, the reduced PCSK9 level in the subject increases less than about 5%, for up to about 9 weeks after administering the PCSK9 antisense oligonucleotide. In one aspect, the reduced PCSK9 level in the subject increases less than about 2%, for up to about 9 weeks after administering the PCSK9 antisense oligonucleotide.

In one aspect, the reduced PCSK9 level in the subject increases less than about 10%, for up to about 12 weeks after administering the PCSK9 antisense oligonucleotide. In one aspect, the reduced PCSK9 level in the subject increases less than about 5%, for up to about 12 weeks after administering the PCSK9 antisense oligonucleotide. In one aspect, the reduced PCSK9 level in the subject increases less than about 2%, for up to about 12 weeks after administering the PCSK9 antisense oligonucleotide.

In one aspect, the level of PCSK9 is reduced at least about 10% after administration of the loading dose. In one aspect, the level of PCSK9 is reduced at least about 20% after administration of the loading dose. In one aspect, the level of PCSK9 is reduced at least about 30% after administration of the loading dose. In one aspect, the level of PCSK9 is reduced at least about 40% after administration of the loading dose. In one aspect, the level of PCSK9 is reduced at least about 50% after administration of the loading dose. In one aspect, the level of PCSK9 is reduced at least about 60% after administration of the loading dose. In one aspect, the level of PCSK9 is reduced at least about 70% after administration of the loading dose. In one aspect, the level of PCSK9 is reduced up to about 80% after administration of the loading dose. In one aspect, the level of PCSK9 is reduced up to about 90% after administration of the loading dose.

In one aspect, the level of PCSK9 in the subject is reduced about 10% to about 95% after administration of the loading dose. In one aspect, the level of PCSK9 in the subject is reduced about 20% to about 80% after administration of the loading dose. In one aspect, the level of PCSK9 in the subject is reduced about 30% to about 70% after administration of the loading dose. In one aspect, the level of PCSK9 in the subject is reduced about 40% to about 70% after administration of the loading dose. In one aspect, the level of PCSK9 in the subject is reduced about 50% to about 70% after administration of the loading dose.

In one aspect, the reduced PCSK9 level in the subject increases less than about 10%, for at least about 2 weeks after administering the loading dose. In one aspect, the reduced PCSK9 level in the subject increases less than about 5%, for at least about 2 weeks after administering the loading dose. In one aspect, the reduced PCSK9 level in the subject increases less than about 2%, for at least about 2 weeks after administering the loading dose.

In one aspect, the reduced PCSK9 level in the subject increases less than about 10%, for at least about 3 weeks after administering the loading dose. In one aspect, the reduced PCSK9 level in the subject increases less than about 5%, for at least about 3 weeks after administering the loading dose. In one aspect, the reduced PCSK9 level in the subject increases less than about 2%, for at least about 3 weeks after administering the loading dose.

In one aspect, the reduced PCSK9 level in the subject increases less than about 10%, for at least about 4 weeks after administering the loading dose. In one aspect, the reduced PCSK9 level in the subject increases less than about 5%, for at least about 4 weeks after administering the loading dose. In one aspect, the reduced PCSK9 level in the subject increases less than about 2%, for at least about 4 weeks after administering the loading dose.

In one aspect, the reduced PCSK9 level in the subject increases less than about 10%, for at least about 5 weeks after administering the loading dose. In one aspect, the reduced PCSK9 level in the subject increases less than about 5%, for at least about 5 weeks after administering the loading dose. In one aspect, the reduced PCSK9 level in the subject increases less than about 2%, for at least about 5 weeks after administering the loading dose.

In one aspect, the reduced PCSK9 level in the subject increases less than about 10%, for up to about 6 weeks after administering the loading dose. In one aspect, the reduced PCSK9 level in the subject increases less than about 5%, for up to about 6 weeks after administering the loading dose. In one aspect, the reduced PCSK9 level in the subject increases less than about 2%, for up to about 6 weeks after administering the loading dose.

In one aspect, the reduced PCSK9 level in the subject increases less than about 10%, for up to about 9 weeks after administering the loading dose. In one aspect, the reduced PCSK9 level in the subject increases less than about 5%, for up to about 9 weeks after administering the loading dose. In one aspect, the reduced PCSK9 level in the subject increases less than about 2%, for up to about 9 weeks after administering the loading dose.

In one aspect, the reduced PCSK9 level in the subject increases less than about 10%, for up to about 12 weeks after administering the loading dose. In one aspect, the reduced PCSK9 level in the subject increases less than about 5%, for up to about 12 weeks after administering the loading dose. In one aspect, the reduced PCSK9 level in the subject increases less than about 2%, for up to about 12 weeks after administering the loading dose.

In one aspect, the reduced PCSK9 level in the subject increases less than about 10%, for at least about 2 weeks after administration of the maintenance dose. In one aspect, the reduced PCSK9 level in the subject increases less than about 5%, for at least about 2 weeks after administration of the maintenance dose. In one aspect, the reduced PCSK9 level in the subject increases less than about 2%, for at least about 2 weeks after administration of the maintenance dose.

In one aspect, the reduced PCSK9 level in the subject increases less than about 10%, for at least about 3 weeks after administration of the maintenance dose. In one aspect, the reduced PCSK9 level in the subject increases less than about 5%, for at least about 3 weeks after administration of the maintenance dose. In one aspect, the reduced PCSK9 level in the subject increases less than about 2%, for at least about 3 weeks after administration of the maintenance dose.

In one aspect, the reduced PCSK9 level in the subject increases less than about 10%, for at least about 4 weeks after administration of the maintenance dose. In one aspect, the reduced PCSK9 level in the subject increases less than about 5%, for at least about 4 weeks after administration of the maintenance dose. In one aspect, the reduced PCSK9 level in the subject increases less than about 2%, for at least about 4 weeks after administration of the maintenance dose.

In one aspect, the reduced PCSK9 level in the subject increases less than about 10%, for at least about 5 weeks after administration of the maintenance dose. In one aspect, the reduced PCSK9 level in the subject increases less than about 5%, for at least about 5 weeks after administration of the maintenance dose. In one aspect, the reduced PCSK9 level in the subject increases less than about 2%, for at least about 5 weeks after administration of the maintenance dose.

In one aspect, the reduced PCSK9 level in the subject increases less than about 10%, for up to about 6 weeks after administration of the maintenance dose. In one aspect, the reduced PCSK9 level in the subject increases less than about 5%, for up to about 6 weeks after administration of the maintenance dose. In one aspect, the reduced PCSK9 level in the subject increases less than about 2%, for up to about 6 weeks after administration of the maintenance dose.

In one aspect, the reduced PCSK9 level in the subject increases less than about 10%, for up to about 9 weeks after administration of the maintenance dose. In one aspect, the reduced PCSK9 level in the subject increases less than about 5%, for up to about 9 weeks after administration of the maintenance dose. In one aspect, the reduced PCSK9 level in the subject increases less than about 2%, for up to about 9 weeks after administration of the maintenance dose.

In one aspect, the reduced PCSK9 level in the subject increases less than about 10%, for up to about 6 weeks after administration of the maintenance dose. In one aspect, the reduced PCSK9 level in the subject increases less than about 5%, for up to about 6 weeks after administration of the maintenance dose. In one aspect, the reduced PCSK9 level in the subject increases less than about 2%, for up to about 6 weeks after administration of the maintenance dose.

Kits

In one aspect, a kit is provided for treating, preventing or ameliorating a cardiovascular disease or one or more symptoms thereof. In one aspect, a kit is provided for reducing a level of PCSK9 in a subject. In one aspect, a kit is provided for reducing PCKS9 expression in a subject. In one aspect, a kit is provided for reducing a serum or plasma level of PCSK9 in a subject. In one aspect, a kit is provided for reducing a LDL-C level in a subject. In one aspect, a kit is provided for reducing a serum or plasma level of LDL-C in a subject.

In one aspect, the kit includes a single dose container that contains a PCSK9 antisense oligonucleotide. In one aspect, the kit includes of a PCSK9 antisense oligonucleotide of Formula I (shown in FIG. 1-3) or a pharmaceutically acceptable salt thereof. In one aspect, the kit includes one or more unit dosages of a PCSK9 antisense oligonucleotide with a nucleobase sequence shown in SEQ ID NO:1. In one aspect, the kit includes one or more unit dosages of a PCSK9 antisense oligonucleotide with a nucleobase sequence consisting of SEQ ID NO: 1.

In one aspect, the kit includes a single dose container that contains from about 10 mg to about 120 mg of a PCSK9 antisense oligonucleotide. In one aspect, the kit includes from about 10 mg to about 120 mg of a PCSK9 antisense oligonucleotide of Formula I (shown in FIG. 1-3) or a pharmaceutically acceptable salt thereof. In one aspect, the kit includes one or more unit dosages of from about 10 mg to about 120 mg of a PCSK9 antisense oligonucleotide with a nucleobase sequence shown in SEQ ID NO:1. In one aspect, the kit includes one or more unit dosages of from about 10 mg to about 120 mg of a PCSK9 antisense oligonucleotide with a nucleobase sequence consisting of SEQ ID NO: 1.

In one aspect, the kit includes a single dose container that contains from about 10 mg to about 120 mg a PCSK9 antisense oligonucleotide that includes a modified oligonucleotide and a conjugate group. In one aspect, the modified oligonucleotide consists of SEQ ID NO:1 and includes: a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment includes a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

In one aspect, the kit includes a unit dosage of about 10 mg, or at least about 20 mg, to about 150 mg of a PCSK9 antisense oligonucleotide. In one aspect, the kit includes a unit dosage of at least about 10 mg, about 15 mg, about 20 mg, about 30 mg, about 40 mg, about 50 mg, about 60 mg, about 70 mg, about 80 mg, or about 90 mg and up to about 100 mg, about 110 mg, about 120 mg, about 130 mg, about 140 mg, or about 150 mg of a PCSK9 antisense oligonucleotide. In one aspect, the kit includes a unit dosage of about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg of a PCSK9 antisense oligonucleotide. In one aspect, the kit includes a unit dosage of about 15 mg, about 50 mg, about 60 mg, about 70 mg, about 80 mg, or about 90 mg of a PCSK9 antisense oligonucleotide.

In one aspect, the kit includes a unit dosage of a PCSK9 antisense oligonucleotide contained in a pre-filled syringe. In one aspect, the kit includes a unit dosage of a PCSK9 oligonucleotide in a single-dose pre-filled syringe.

Method of Treatment

In one aspect, a method is provided for reducing a level of PCSK9 in a subject. In one aspect, a method is provided for reducing or inhibiting expression PCSK9 in a subject. In one aspect, a method is provided for reducing a serum or plasma level of PCSK9 in a subject. In one aspect, therapeutic and prophylactic methods are provided for treating or preventing a disease, disorder or condition associated with PCSK9. In one aspect, therapeutic and prophylactic methods are provided for treating or preventing a disease, disorder or condition by reducing a level of PCSK9 in a subject. In one aspect, therapeutic and prophylactic methods are provided for treating or preventing a disease, disorder or condition by reducing PCSK9 expression in a subject. In one aspect, the subject is a human.

PCSK9 regulates the levels of LDL receptor present in a subject, which is responsible for removing cholesterol-rich LDL particles from the plasma. In one aspect, reducing the level of PCSK9 in a subject results in a decrease in LDL-C levels in the blood or serum of the subject. In one aspect, a method is provided for reducing a level of LDL-C in a subject. In one aspect, a method is provided for reducing a serum or plasma level of LDL-C in a subject. In one aspect, a method is provided for reducing a risk of cardiovascular disease in a subject.

In one aspect, the disease associated with PCSK9 is selected from: elevated low-density lipoprotein cholesterol (LDL-C) levels, hyperlipidemia, dyslipidemia, atherosclerosis, cardiovascular disease, hypercholesterolemia, and combinations thereof. In one aspect, the disease associated with PCSK9 is dyslipidemia or hypercholesterolemia.

PSCK9

In one aspect, a level of PCSK9 in the subject is reduced. In one aspect, the method includes administering a PCSK9 antisense oligonucleotide. In one aspect, the level of PCSK9 in the subject is reduced about 10% to about 95% after administering a PCSK9 antisense oligonucleotide as compared to a level of PCSK9 in the subject before administering the PCSK9 antisense oligonucleotide. dosage. In one aspect, the level of PCSK9 in the subject is reduced about 10%, about 20%, about 30%, about 40%, or about 50% to about 60%, about 70%, about 80%, or about 90% after administration of the PCSK9 antisense oligonucleotide as compared to a level of PCSK9 in the subject before administration of the PCSK9 antisense oligonucleotide. In one aspect, the level of PCSK9 in the subject is reduced about 10% to about 95%, about 20% to about 80%, about 30% to about 70%, about 40% to about 70%, or about 50% to about 70% after administration of the PCSK9 antisense oligonucleotide as compared to a level of PCSK9 in the subject before administration of the PCSK9 antisense oligonucleotide.

In one aspect, the level of PCSK9 in the subject is reduced for at least about 2 weeks after administering the PCSK9 antisense oligonucleotide as compared to a level of PCSK9 in the subject before administration of the PCSK9 antisense oligonucleotide. In one aspect, the level of PCSK9 in the subject is reduced for at least about 3 weeks, at least about 4 weeks, or at least about 5 weeks and up to about 6 weeks, about 9 weeks or about 12 weeks after administration of the PCSK9 antisense oligonucleotide as compared to a level of PCSK9 in the subject before administration of the PCSK9 antisense oligonucleotide.

In one aspect, a circulating level of PCSK9 in the subject is reduced. In one aspect, a plasma level of PCSK9 in the subject is reduced. In one aspect, a serum level of PCSK9 in the subject is reduced. In one aspect, PCSK9 expression is reduced.

In one aspect, a method is provided for treating, preventing or ameliorating a cardiovascular disease. In one aspect, a method is provided for treating, preventing or ameliorating lipidemia, including, for example, hyperlipidemia or other disease, disorder or conditions associated with lipid imbalance such as hypercholesterolemia, hypertriglyceridemia and conditions associated with these disorders, including, for example, heart and circulatory diseases.

In one aspect, a method is provided for treating a subject that has, or is at risk of developing, a disease associated with PCSK9, including, for example, hypercholesterolemia or a related disorder (e.g., atherosclerosis), and administering to the subject, a unit dosage that includes a PCSK9 antisense oligonucleotide. In one aspect, the PCSK9 antisense oligonucleotide is administered in connection with another therapeutic agent, for example, a statin. In one aspect, the PCSK9 antisense oligonucleotide is not administered in connection with another therapeutic agent, such as a statin.

In one aspect, a method is provided for treating a disease associated with PCSK9 in a subject. In one aspect, the method includes administering to the subject a unit dosage that includes from about 20 to about 120 mg of a PCSK9 antisense oligonucleotide of Formula I, or pharmaceutically acceptable salt thereof to the subject, for example about 50 mg of a PCSK9 antisense oligonucleotide of Formula I. In one aspect, the method includes administering a unit dosage that includes from about 10 to about 120 mg of a PCSK9 antisense oligonucleotide with a nucleobase sequence of SEQ ID NO: 1 to the subject, for example about 50 mg of a PCSK9 antisense oligonucleotide of Formula I. In one aspect, the method includes administering a unit dosage that includes from about 10 to about 120 mg of a PCSK9 antisense oligonucleotide consisting of a nucleobase sequence of SEQ ID NO: 1 to the subject, for example about 50 mg of a PCSK9 antisense oligonucleotide of Formula I.

In one aspect, the method includes administering a unit dosage that includes about 10 mg to about 120 mg of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment. In one aspect, each nucleoside of each wing segment comprises a 2′-O-ethyl (cEt) sugar. In one aspect, each internucleoside linkage is a phosphorothioate linkage. In one aspect, each cytosine is a 5-methylcytosine. In one aspect, the conjugate group includes 5′-Trishexylamino-(THA)-C₆GalNAC3 linked to the 5′ end of the modified oligonucleotide.

LDL-C

In one aspect, a method is provided for reducing a low-density lipoprotein cholesterol (LDL-C) level in a subject. In one aspect, the method includes administering to the subject a unit dosage that includes from about 10 mg, or at least about 20 mg, to about 120 mg of a PCSK9 antisense oligonucleotide of Formula I, or pharmaceutically acceptable salt thereof. In one aspect, the method includes administering to the subject a unit dosage that includes from about 10 mg, or at least about 20 mg, to about 120 mg of a PCSK9 antisense oligonucleotide with a nucleobase sequence of SEQ ID NO:1. In one aspect, the method includes administering to the subject a unit dosage that includes from about 10 mg, or at least about 20 mg, to about 120 mg of a PCSK9 antisense oligonucleotide with a nucleobase sequence consisting of SEQ ID NO:1, for example about 50 mg of a PCSK9 antisense oligonucleotide a PCSK9 antisense oligonucleotide with a nucleobase sequence consisting of SEQ ID NO:1.

In one aspect, the method includes administering to the subject a unit dosage that includes from about 10 mg, or at least about 20 mg, or at least about 50 mg, to about 120 mg of a PCSK9 antisense oligonucleotide that includes a modified oligonucleotide and a conjugate group, or pharmaceutically acceptable salt thereof. In one aspect, the modified oligonucleotide consists of SEQ ID NO:1 and includes a gap segment consisting of ten linked deoxynucleosides; a 5′ wing segment consisting of three linked nucleosides; and a 3′ wing segment consisting of three linked nucleosides. In one aspect, the gap segment is positioned between the 5′ wing segment and the 3′ wing segment; wherein each nucleoside of each wing segment comprises a 2′-O-ethyl (cEt) sugar;

wherein each internucleoside linkage is a phosphorothioate linkage; and wherein each cytosine is a 5-methylcytosine. In one aspect, the conjugate group comprises 5′-Trishexylamino-(THA)-C₆GalNAC3 linked to the 5′ end of the modified oligonucleotide.

In one aspect, the level of LDL-C in the subject is reduced about 10% to about 90% after administration of the PCSK9 antisense oligonucleotide as compared to a level of LDL-C in the subject before administration of the PCSK9 antisense oligonucleotide. In one aspect, the level of LDL-C in the subject is reduced at least about 10%, about 20%, about 30%, about 40%, about 50%, about 60%, or about 70%, and up to about 80%, or about 90% after administration of the PCSK9 antisense oligonucleotide as compared to a level of LDL-C in the subject before administration of the PCSK9 antisense oligonucleotide. In one aspect, the level of LDL-C in the subject is reduced about 20% to about 80%, about 30% to about 70%, about 40% to about 70%, or about 50% to about 70% after administration of the PCSK9 antisense oligonucleotide as compared to a level of LDL-C in the subject before administration of the PCSK9 antisense oligonucleotide.

In one aspect, the level of LDL-C in the subject is reduced for at least about 2 weeks after administering the PCSK9 antisense oligonucleotide as compared to a level of LDL-C in the subject before administration of the PCSK9 antisense oligonucleotide. In one aspect, the level of LDL-C in the subject is reduced for at least about 3 weeks, about 4 weeks, or about 5 weeks and up to about 6 weeks, 9 weeks or 12 weeks after administering the PCSK9 antisense oligonucleotide as compared to a level of LDL-C in the subject before administration of the PCSK9 antisense oligonucleotide.

In one aspect, a circulating level of LDL-C of the subject is reduced. In one aspect, a serum level of LDL-C of the subject is reduced. In one aspect, a plasma level of LDL-C of the subject is reduced.

In one aspect, the subject has a serum low-density lipoprotein cholesterol (LDL-C) level of at least about 100 mg/dL, at least about 110 mg/dL, at least about 120 mg/dL, at least about 130 mg/dL, at least about 140 mg/dL, at least about 150 mg/dL, at least about 160 mg/dL, at least about 170 mg/dL, at least about 180 mg/dL, at least about 190 mg/dL, at least about 200 mg/dL, at least about 300 mg/dL or at least about 400 mg/dL prior to administration of the PCSK9 antisense oligonucleotide.

In one aspect, the unit dosage is administered to the subject about every 2 weeks to about every 6 weeks. In one aspect, the unit dosage is administered to the subject about every 2 weeks, about every 3 weeks, about every 4 weeks, about every 5 weeks or about every 6 weeks, for at least about 6 weeks, at least about 12 weeks, at least about 26 weeks, or at least about 52 weeks. In one aspect, the unit dosage is administered to the subject once a month for at least about 3 months. In one aspect, the unit dosage is administered to the subject once a month for at least about at least about 6 months, at least about 9 months, at least about 12 months, at least about 15 months or at least about 24 months. In one aspect, the unit dosage is administered to the subject once every other month for at least about 4 months. In one aspect, the unit dosage is administered to the subject once every other month for at least about at least about 6 months, at least about 12 months, at least about 18 months, at least about 24 months or at least about 36 months. In one aspect, the unit dosage is administered to the subject about every 21 days, about every 28 days, or about every 30 days for at least about 90 days, at least about 120 days, at least about 180 days, or at least about 365 days.

In one aspect, the unit dosage is administered by parenteral administration. In one aspect, parenteral administration includes subcutaneous, intravenous, or intramuscular administration. In one aspect, parenteral administration includes subcutaneous administration.

In one aspect, the subject is a human.

| SEQUENCES | |
|---|---|
| Synthetic Oligonucleotide | |
| SEQ ID NO: 1 | |
| aataatctca tgtcag | |
| Homo Sapiens proprotein convertase | |
| subtilisin/kexin type 9 | |
| (PCSK9) transcript variant 1, mRNA | |
| RefSeq Accession No. NM_174936.3 | |
| SEQ ID NO: 2 | |
| 1 | gtccgatggg gctctggtgg cgtgatctgc |
| | gcgccccagg cgtcaagcac ccacacccta |
| 61 | gaaggtttcc gcagcgacgt cgaggcgctc |
| | atggttgcag gcgggcgccg ccgttcagtt |
| 121 | cagggtctga gcctggagga gtgagccagg |
| | cagtgagact ggctcgggcg ggccgggacg |
| 181 | cgtcgttgca gcagcggctc ccagctccca |
| | gccaggattc cgcgcgcccc ttcacgcgcc |
| 241 | ctgctcctga acttcagctc ctgcacagtc |
| | ctccccaccg caaggctcaa ggcgccgccg |
| 301 | gcgtggaccg cgcacggcct ctaggtctcc |
| | tcgccaggac agcaacctct cccctggccc |
| 361 | tcatgggcac cgtcagctcc aggcggtcct |
| | ggtggccgct gccactgctg ctgctgctgc |
| 421 | tgctgctcct gggtcccgcg ggcgcccgtg |
| | cgcaggagga cgaggacggc gactacgagg |
| 481 | agctggtgct agccttgcgt tccgaggagg |
| | acggcctggc cgaagcaccc gagcacggaa |
| 541 | ccacagccac cttccaccgc tgcgccaagg |
| | atccgtggag gttgcctggc acctacgtgg |
| 601 | tggtgctgaa ggaggagacc cacctctcgc |
| | agtcagagcg cactgcccgc cgcctgcagg |
| 661 | cccaggctgc ccgccgggga tacctcacca |
| | agatcctgca tgtcttccat ggccttcttc |
| 721 | ctggcttcct ggtgaagaty agtggcgacc |
| | tgctggagct ggccttgaag ttgccccatg |
| 781 | tcgactacat cgaggaggac tcctctgtct |
| | ttgcccagag catcccgtgg aacctggagc |
| 841 | ggattacccc tccacggtac cgggcggatg |
| | aataccagcc ccccgacgga ggcagcctgg |
| 901 | tggaggtgta tctcctagac accagcatac |
| | agagtgacca ccgggaaatc gagggcaggg |
| 961 | tcatggtcac cgacttcgag aatgtgcccg |
| | aggaggacgg gacccgcttc cacagacagg |
| 1021 | ccagcaagtg tgacagtcat ggcacccacc |
| | tggcaggggt ggtcagcggc cgggatgccg |
| 1081 | gcgtggccaa gggtgccagc atgcgcagcc |
| | tgcgcgtgct caactgccaa gggaagggca |
| 1141 | cggttagcgg caccctcata ggcctggagt |
| | ttattcggaa aagccagctg gtccagcctg |
| 1201 | tggggccact ggtggtgctg ctgcccctgg |
| | cgggtgggta cagccgcgtc ctcaacgccg |
| 1261 | cctgccagcg cctggcgagg gctggggtcg |
| | tgctggtcac cgctgccggc aacttccggg |
| 1321 | acgatgcctg cctctactcc ccagcctcag |
| | ctcccgaggt catcacagtt ggggccacca |
| 1381 | atgcccaaga ccagccggtg accctgggga |
| | ctttggggac caactttggc cgctgtgtgg |
| 1441 | acctctttgc cccaggggag gacatcattg |
| | gtgcctccag cgactgcagc acctgctttg |
| 1501 | tgtcacagag tcggacatca caggctgctg |
| | cccacgtggc tggcattgca gccatgatgc |
| 1561 | tgtctgccga gccggagctc accctggccg |
| | agttgaggca gagactgatc cacttctctg |
| 1621 | ccaaagatgt catcaatgag gcctggttcc |
| | ctgaggacca gcgggtactg acccccaacc |
| 1681 | tggtggccgc cctgcccccc agcacccatg |
| | gggcaggttg gcagctgttt tgcaggactg |
| 1741 | tatggtcagc acactcgggg cctacacgga |
| | tggccacagc cgtcgcccgc tgcgccccag |
| 1801 | atgaggagct gctgagctgc tccagtttct |
| | ccaggagtgg gaagcggcgg ggcgagcgca |
| 1861 | tggaggccca agggggcaag ctggtctgcc |
| | gggcccacaa cgcttttggg ggtgagggtg |
| 1921 | tctacgccat tgccaggtgc tgcctgctac |
| | cccaggccaa ctgcagcgtc cacacagctc |
| 1981 | caccagctga ggccagcatg gggacccgtg |
| | tccactgcca ccaacagggc cacgtcctca |
| 2041 | caggctgcag ctcccactgg gaggtggagg |
| | accttggcac ccacaagccg cctgtgctga |
| 2101 | ggccacgagg tcagcccaac cagtgcgtgg |
| | gccacaggga ggccagcatc cacgcttcct |
| 2161 | gctgccatgc cccaggtctg gaatgcaaag |
| | tcaaggagca tggaatcccg gcccctcagg |
| 2221 | agcaggtgac cgtggcctgc gaggagggct |
| | ggaccctgac tggctgcagt gccctccctg |
| 2281 | ggacctccca cgtcctgggg gcctacgccg |
| | tagacaacac gtgtgtagtc aggagccggg |
| 2341 | acgtcagcac tacaggcagc accagcgaag |
| | gggccgtgac agccgttgcc atctgctgcc |
| 2401 | ggagccggca cctggcgcag gcctcccagg |
| | agctccagtg acagccccat cccaggatgg |
| 2461 | gtgtctgggg agggtcaagg gctggggctg |
| | agctttaaaa tggttccgac ttgtccctct |
| 2521 | ctcagccctc catggcctgg cacgagggga |
| | tggggatgct tccgcctttc cggggctgct |
| 2581 | ggcctggccc ttgagtgggg cagcctcctt |
| | gcctggaact cactcactct gggtgcctcc |
| 2641 | tccccaggtg gaggtgccag gaagctccct |
| | ccctcactgt ggggcatttc accattcaaa |
| 2701 | caggtcgagc tgtgctcggg tgctgccagc |
| | tgctcccaat gtgccgatgt ccgtgggcag |
| 2761 | aatgactttt attgagctct tgttccgtgc |
| | caggcattca atcctcaggt ctccaccaag |
| 2821 | gaggcaggat tcttcccatg gataggggag |
| | ggggcggtag gggctgcagg gacaaacatc |
| 2881 | gttggggggt gagtgtgaaa ggtgctgatg |
| | gccctcatct ccagctaact gtggagaagc |
| 2941 | ccctgggggc tccctgatta atggaggctt |
| | agctttctgg atggcatcta gccagaggct |
| 3001 | ggagacaggt gcgcccctgg tggtcacagg |
| | ctgtgccttg gtttcctgag ccacctttac |
| 3061 | tctgctctat gccaggctgt gctagcaaca |
| | cccaaaggtg gcctgcgggg agccatcacc |
| 3121 | taggactgac tcggcagtgt gcagtggtgc |
| | atgcactgtc tcagccaacc cgctccacta |
| 3181 | cccggcaggg tacacattcg cacccctact |
| | tcacagagga agaaacctgg aaccagaggg |
| 3241 | ggcgtgcctg ccaagctcac acagcaggaa |
| | ctgagccaga aacgcagatt gggctggctc |
| 3301 | tgaagccaag cctcttctta cttcacccgg |
| | ctgggctcct catttttacg ggtaacagtg |
| 3361 | aggctgggaa ggggaacaca gaccaggaag |
| | ctcggtgagt gatggcagaa cgatgcctgc |
| 3421 | aggcatggaa ctttttccgt tatcacccag |
| | gcctgattca ctggcctggc ggagatgctt |
| 3481 | ctaaggcatg gtcgggggag agggccaaca |
| | actgtccctc cttgagcacc agccccaccc |
| 3541 | aagcaagcag acatttatct tttgggtctg |
| | tcctctctgt tgccttttta cagccaactt |
| 3601 | ttctagacct gttttgcttt tgtaacttga |
| | agatatttat tctgggtttt gtagcatttt |
| 3721 | tattaatatg gtgacttttt aaaataaaaa |
| | caaacaaacg ttgtcctaac aaaaaaaaaa |
| 3661 | aaaaaaaaaa a |

### EXAMPLES

### Example 1. Repatha Efficacy and Durability Vs AZD8233 Administration

### Example 1A. Effect of One Missed Repatha® Dose on LDL-C

Repatha® (Evolocumab) is a human monoclonal antibody that inhibits PCSK9 that has been approved by the Food and Drug Administration (FDA) for the treatment of patients with familial hypercholesterolemia. Repatha® can be dosed every 2 weeks using a single-use prefilled autoinjector or once a month using a single-use on-body infusor with a prefilled cartridge.

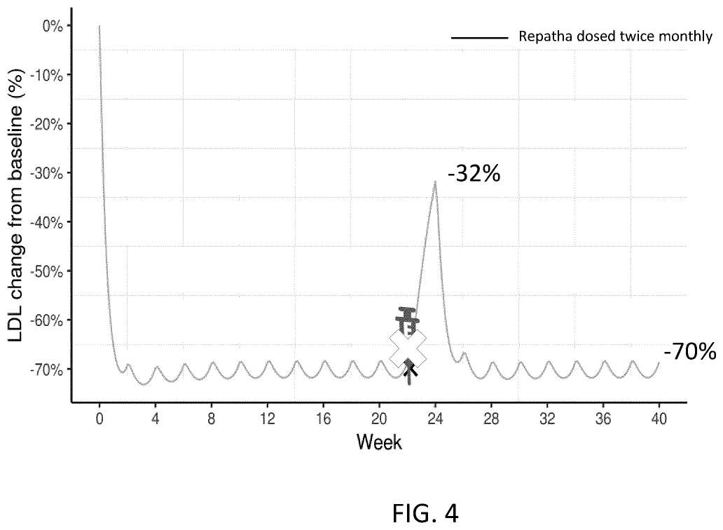

FIG. 4 shows the pharmacokinetic/pharmacodynamic (PK/PD) profile for Repatha®. Notably, the monoclonal antibody has a low tolerance for missed doses, with a loss of 32% of the LDL change from baseline after 1 missed dose.

### Example 1B. LDL-C Reduction of AZD8233 vs. Repatha®

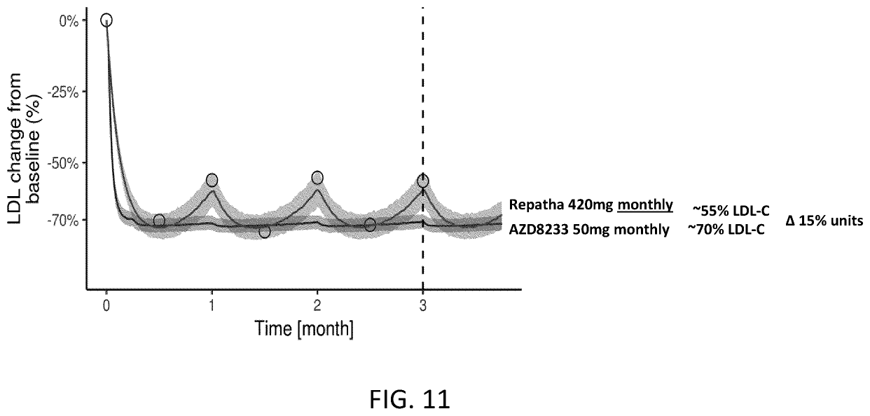

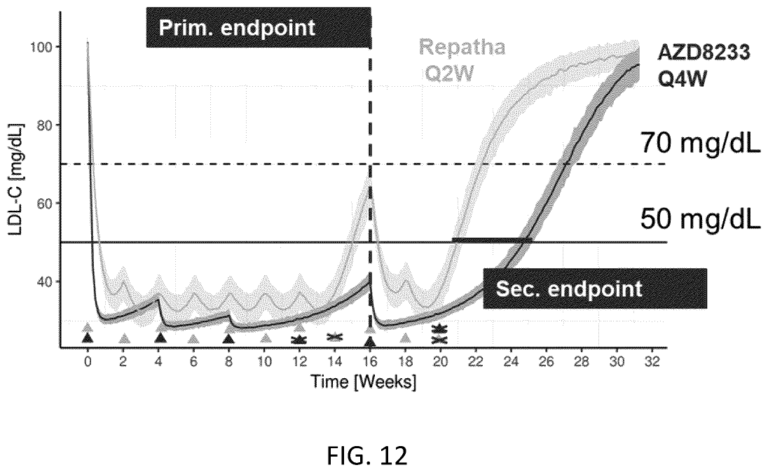

FIG. 11 shows simulations of LDL-C change from baseline (%) profiles for Repatha® (420 mg monthly) versus AZD8233 (50 mg monthly). AZD8233 showed approximately a 70% reduction in LDL-C from baseline vs. Repatha® which showed approximately a 55% reduction in LDL-C from baseline. FIG. 12 shows simulations of LDL-C reduction after one missed dose with Repatha® administered twice monthly at 140 mg (LDL-C reduction approximately 37%) vs AZD8233 administered monthly at 50 mg (LDL-C reduction approximately 60%). Time to LDL-C outside of target range (>55 mg/dL) when not adherent is 2 weeks for Repatha® administered twice monthly at 140 mg and 6 weeks for AZD8233 administered monthly at 50 mg. All predictions based on simulations using published Repatha model and AZD8233 model developed on SAD and literature data.

### Example 2. Comparison of One Missed Dose of AZD8233 Compared to Inclisiran® on LDL-C

Inclisiran® is a double stranded small interfering RNA molecule (siRNA) conjugated on the sense strand with triantennary N-acetylgalactosamine (GalNAc) that has been approved by the FDA for treatment of hypercholesterolemia. Inclisiran® increases LDL-C receptor recycling and expression on hepatocyte cell surfaces to increase LDL-C update and reduce LDL-C levels in circulation. Inclisiran® is administered by a physician subcutaneously with an initial dose, again at 3 months and then every 6 months.

AZD8233 is administered subcutaneously once a month at home, by the patient.

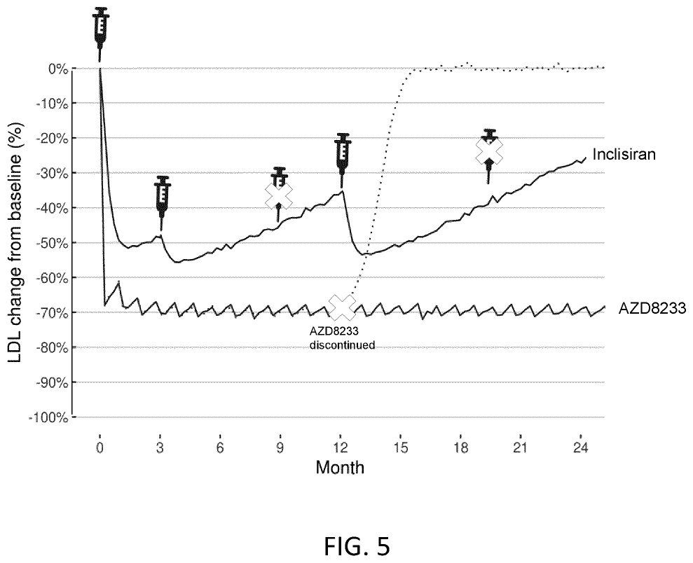

FIG. 5 shows the PK/PD profile for Inclisiran® and AZD8233. A 70% reduction in LDL-C from baseline is obtained after the initial dose of AZD8233. Notably, there is a minimal impact of a missed dose of AZD8233 on LDL-C change from baseline. In contrast, Inclisiran® provides only about a 50% reduction in LDL-C after three months from baseline after the initial dose showing that efficacy is sensitive to a missed dose.

### Example 3. Relative Risk Reduction for AZD8233 Compared to Inclisiran®

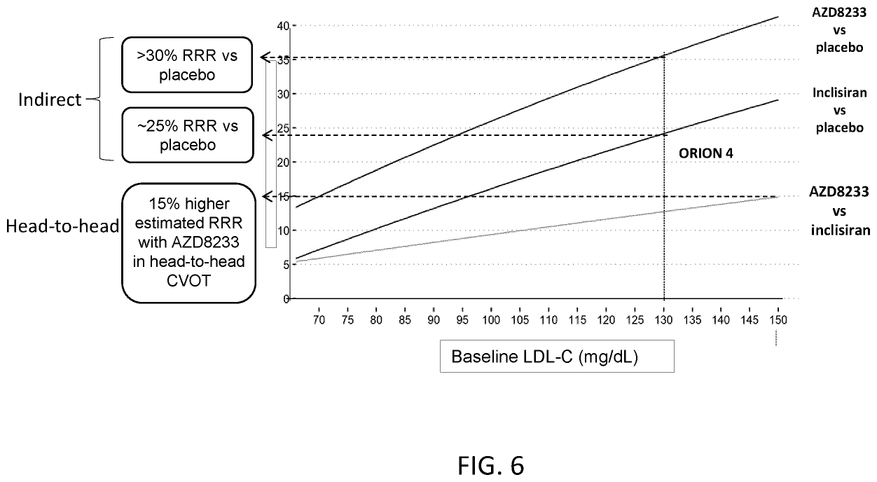

FIG. 6 shows the Relative Risk Reduction (RRR) of AZD8233 versus placebo; Inclisiran® versus placebo; and AZD8233 versus Inclisiran®. In an indirect comparison, AZD8233 provided a 70% reduction in LDL-C and a at least 30% RRR as compared to placebo, while Inclisiran® provided a 51% reduction in LDL-C and approximately a 25% RRR as compared to placebo. In a head to head comparison, AZD8233 reduced LDL-C by 19% more than Inclisiran® and provided a 15% greater estimated RRR than Inclisiran® in a Cardiovascular Outcome Trial (CVOT). The slopes in FIG. 3 apply to secondary prevention. All scenarios are in addition to administration of a combination therapy with a statin and Ezetimibe.

### Example 4. AZD8233 Single Ascending Dose (SAD) Study

A randomized, single-blind, placebo-controlled, single ascending dose study was performed in humans to evaluate the safety, tolerability, pharmacokinetics (PK) and pharmacodynamics (PD) of single doses of AZD8233 in subjects with LDL-C ≥100 mg/dL.

Briefly, AZD8233 was administered subcutaneously (SC) to 56 subjects enrolled in 7 cohorts (6 active and 2 placebo in each cohort). The doses studied ranged from 4 to 120 mg. Serial blood samples were collected to assess PK, PCSK9 and LDL-C profiles after administration of AZD8233. PK parameters were calculated using non-compartmental methods.

After SC administration, AZD8233 was generally well tolerated, with no clinically relevant safety and tolerability findings observed and no serious adverse events. Peak plasma concentrations were achieved within 1 to 3 hours after dosing. After peaking, plasma concentrations declined biphasically with a terminal half-life of 2 weeks to 3 weeks. A dose dependent decrease in plasma levels of PCSK9 and LDL-C was observed. The largest mean percent reductions from baseline were >90% for PCSK9 and 70% for LDL-C. PCSK9 and LDL-C levels slowly returned to baseline or close to baseline levels over the 16 weeks post dose follow up period. This data suggests that a high level of PCSK9 and LDL-C reduction can be maintained over a dose interval of once monthly or less frequent at a dose below 100 mg.

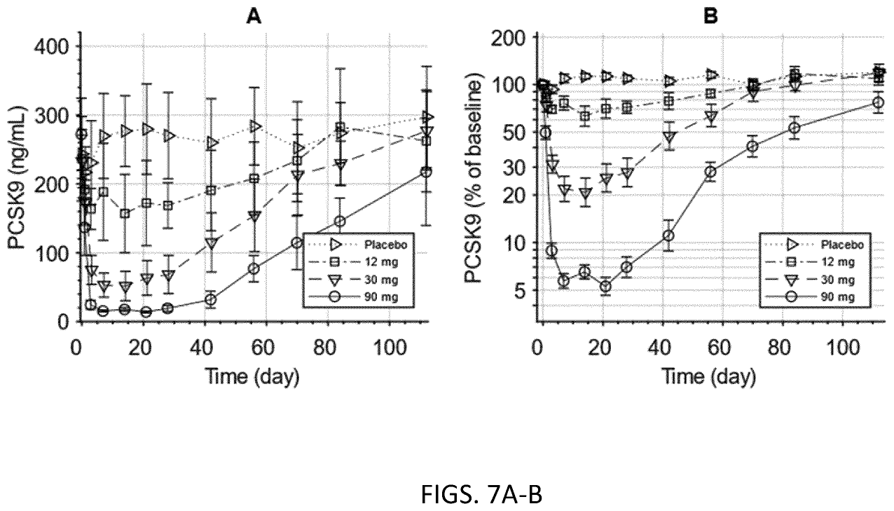

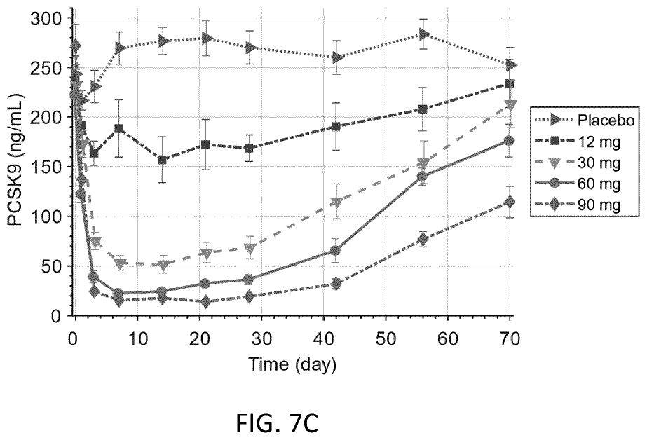

FIG. 7A shows the geometric mean and standard error of the mean (SEM) for PCSK9 levels for AZD8233 upon a single dose (12 mg, 30 mg, and 90 mg) of AZD8233 to humans with elevated LDL-C levels. FIG. 7B shows the same data as FIG. 7A but baseline-corrected at logarithmic scale. N=6 per treated group and N=14 for placebo. Single doses of AZD8233 potently and dose-dependently reduced plasma PCSK9 protein levels in humans with elevated LDL-C levels at 134±3.9 mg/dL (mean±SEM). For the 90 mg dose, circulating PCSK9 was reduced by up to 95%, and remained reduced to more than 90% over at least one month.

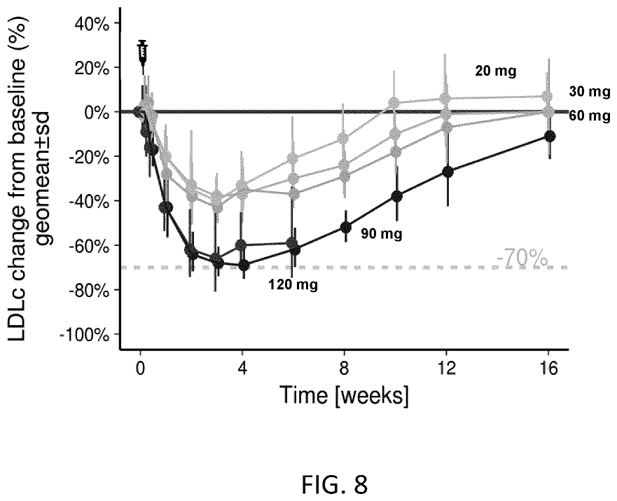

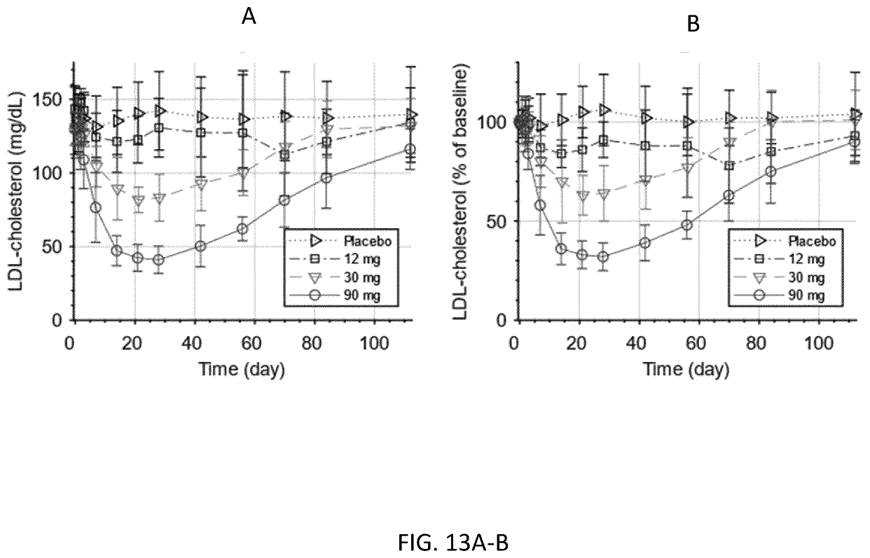

FIG. 8 shows the geometric mean and standard deviation (SD) for LDL-C change from baseline (%) for AZD8233 in a single ascending dose (SAD) study for doses of 20 mg, 30 mg, 60 mg, 90 mg and 120 mg (N=6 per cohort). A 69% LDL-C reduction from baseline was observed the 90 mg and 120 mg doses (95% confidence interval (CI) −65%, −73%). The 90 mg week 4 dose was placebo corrected. Data for placebo, 4 mg and 12 mg doses is not shown. FIG. 13A and FIG. 13B further show LDL-C change (FIG. 13A illustrates measured data and FIG. 13B illustrates baseline-corrected data) in a single ascending dose (SAD) study for doses of 12 mg, 30 mg, and 90 mg (N=6 per cohort).

### Example 5. LDL-C Efficacy of AZD8233 Compared to Inclisiran®

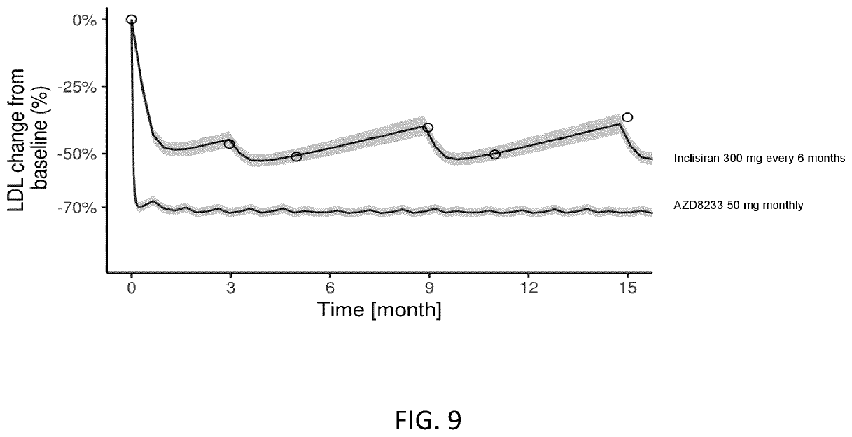

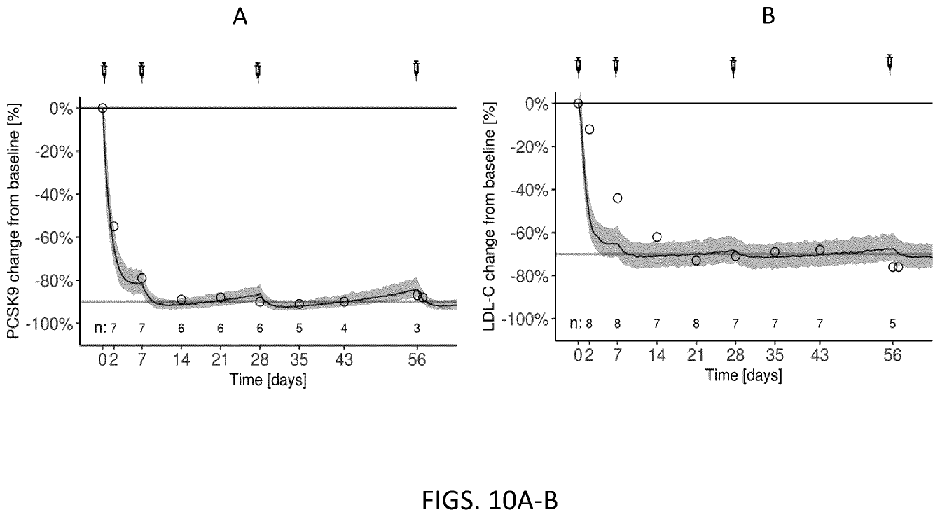

FIG. 9. Shows simulations (median and 90% confidence interval) of LDL-C steady state profiles for Inclisiran® (300 mg every 6 months) versus AZD8233 (50 mg monthly). AZD8233 showed approximately a 70% reduction in LDL-C from baseline, with a variability of less than ±5% (A LDL-C±5%). Inclisiran® showed approximately a 50% reduction in LDL-C from baseline, with a variability of approximately 18% (Δ LDL-C 18%).

### Example 6. AZD8233 ETESIAN Ph2B Dose-Ranging Study

A randomized parallel, double-blind, placebo-controlled, dose-ranging Ph2b study was performed in humans to evaluate the effect of AZD8233 on LDL-C across different dose levels in subjects with a fasting LDL-C of ≥70 and <190 mg/dL, fasting triglycerides of <400 mg/dL, who were receiving moderate- or high-intensity statin therapy and were on stable medication for ≥3 months prior to screening with no planned medication or dose changes. Participants were randomized 1:1:1:1 to receive subcutaneous injections over a 12-week period (days 1, 8, 29 and 57) of either AZD8233 90 mg, 50 mg, 15 mg; or placebo, and were followed-up for up to 16 weeks post last dose. The primary objective of ETESIAN was to assess the effect of AZD8233 doses on LDL-C levels versus placebo at week 12; circulating LDL-C was measured directly using plasma samples. Planned enrollment was for approximately 108 patients to allow for at least 80 evaluable patients to have completed treatment by week 12; 119 patients were enrolled. Absolute change from baseline in log-transformed LDL-C and PCSK9 levels were analyzed using a mixed model for repeated measures with baseline as a covariate and treatment, time and interaction between treatment and time as factors.

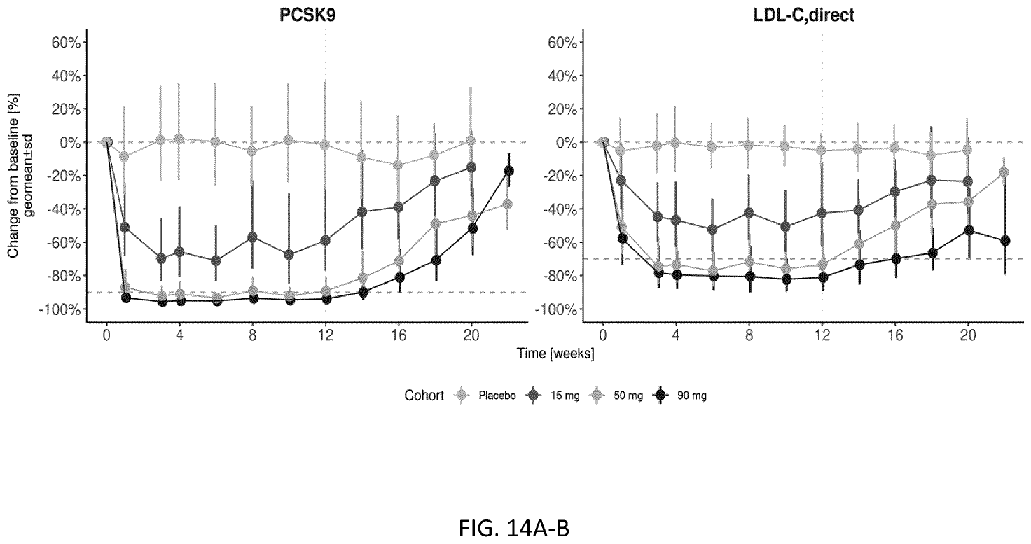

FIGS. 14A-B shows the change in circulating PCSK9 and LDL-C (geometric mean and 95% CI) in humans with elevated LDL-C levels upon varied dosing (15 mg, 50 mg, 90 mg) of AZD8233. FIG. 14A illustrates the baseline-corrected data for circulating PCSK9 levels and FIG. 14B illustrates the baseline-corrected data for LDL-C levels (N=29-30 per treated group and N=30 for placebo). At a dose of 50 mg per month, circulating PCSK9 levels were reduced by 88% at week 12 (95% CI −91, −84) and circulating LDL-C levels were reduced by 72% at week 12 (95% CI −78, −65).

What is claimed is:

1. A unit dosage comprising from about 10 mg to about 120 mg of a compound of Formula I (SEQ ID NO: 1) or a pharmaceutically acceptable salt thereof:

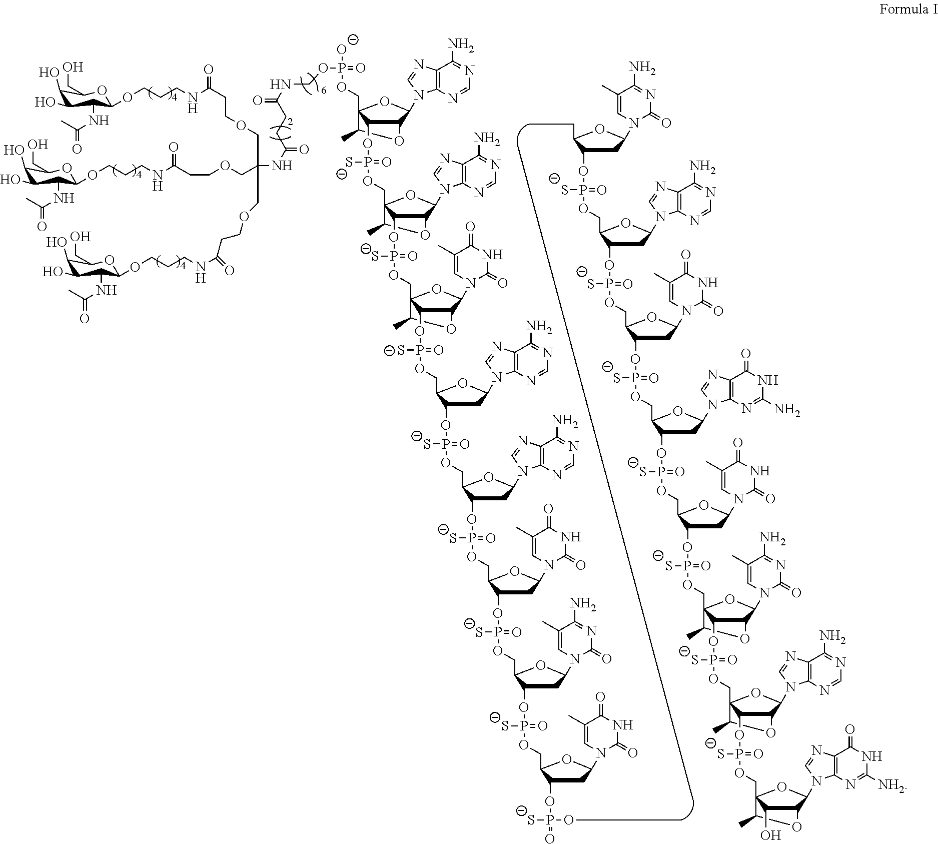

2. A unit dosage comprising from about 10 mg to about 120 mg of a compound or pharmaceutically acceptable salt thereof, wherein the compound comprises a modified oligonucleotide and a conjugate group, wherein the modified oligonucleotide consists of SEQ ID NO:1 and comprises:
10 a gap segment consisting of ten linked deoxynucleosides;
a 5′ wing segment consisting of three linked nucleosides; and
a 3′ wing segment consisting of three linked nucleosides,
wherein the gap segment is positioned between the 5′ wing segment and the 3′ wing segment; wherein each nucleoside of each wing segment comprises a 2′-O-ethyl (cEt) sugar; wherein each internucleoside linkage is a phosphorothioate linkage; and wherein each cytosine is a 5-methylcytosine.

3. The unit dosage of claim 2, wherein the conjugate group comprises 5′-Trishexylamino-(THA)-C₆GalNAC3 linked to the 5′ end of the modified oligonucleotide.

4. The unit dosage of any of the preceding claims, comprising at least about 15 mg, about 20 mg, about 30 mg, about 40 mg or about 50 mg, about 60 mg, about 70 mg, about 80 mg or about 90 mg and up to about 100 mg, about 110 mg or about 120 mg of the compound or a pharmaceutically acceptable salt thereof

5. The unit dosage of any of the preceding claims, comprising about 15 mg, 20 mg, about 30 mg, about 40 mg, about 50 mg, about 60 mg, about 70 mg, about 80 mg, about 90 mg, about 100 mg, about 110 mg or about 120 mg of the compound or a pharmaceutically acceptable salt thereof.

6. The unit dosage of any of the preceding claims, comprising from about 50 mg to about 100 mg of the compound or a pharmaceutically acceptable salt thereof.

7. The unit dosage of any of the preceding claims, comprising about 50 mg, about 70 mg or about 90 mg of the compound or a pharmaceutically acceptable salt thereof.

8. The unit dosage of any of the preceding claims, formulated for parenteral administration.

9. The unit dosage of claim 8, wherein parenteral administration comprises subcutaneous, intramuscular or intravenous administration.

10. The unit dosage of claim 8 or 9, formulated for subcutaneous administration.

11. The unit dosage of any of the preceding claims, wherein the pharmaceutically acceptable salt is a sodium salt.

12. The unit dosage of any of the preceding claims, wherein the pharmaceutically acceptable salt is a potassium salt.

13. The unit dosage of any of the preceding claims, further comprising a pharmaceutically acceptable carrier, adjuvant or excipient.

14. The unit dosage of any of the preceding claims, contained in a pre-filled syringe.

15. The unit dosage of any of the preceding claims, contained in a single-dose pre-filled syringe.

16. A method of reducing a low-density lipoprotein cholesterol (LDL-C) in a subject, the method comprising administering the unit dosage of any of claims 1 to 15 to the subject.

17. A method of reducing a level of PCSK9 in a subject, the method comprising administering the unit dosage of any of claims 1 to 15 to the subject.

18. A method of reducing a risk of cardiovascular disease in a subject, the method comprising administering the unit dosage of any of claims 1 to 15 to the subject.

19. Use of a unit dose according to any of claims 1 to 15, in the manufacture of a medicament for the reduction of a low-density lipoprotein cholesterol (LDL-C) level in a subject.

20. Use of a unit dose according to any of claims 1 to 15, in the manufacture of a medicament for the treatment of disease associated with PCSK9.

21. The use of claim 20, wherein the disease associated with PCSK9 is selected from: elevated low-density lipoprotein cholesterol (LDL-C) levels, hyperlipidemia, dyslipidemia, atherosclerosis, cardiovascular disease, hypercholesterolemia, and combinations thereof.

22. The use of claim 20 or 21, wherein the disease associated with PCSK9 is dyslipidemia or hypercholesterolemia.

23. A method of reducing a low-density lipoprotein cholesterol (LDL-C) level in a subject, comprising administering to the subject a unit dosage comprising from about 10 to about 120 mg of a compound of Formula I (SEQ ID NO:1), or pharmaceutically acceptable salt thereof:

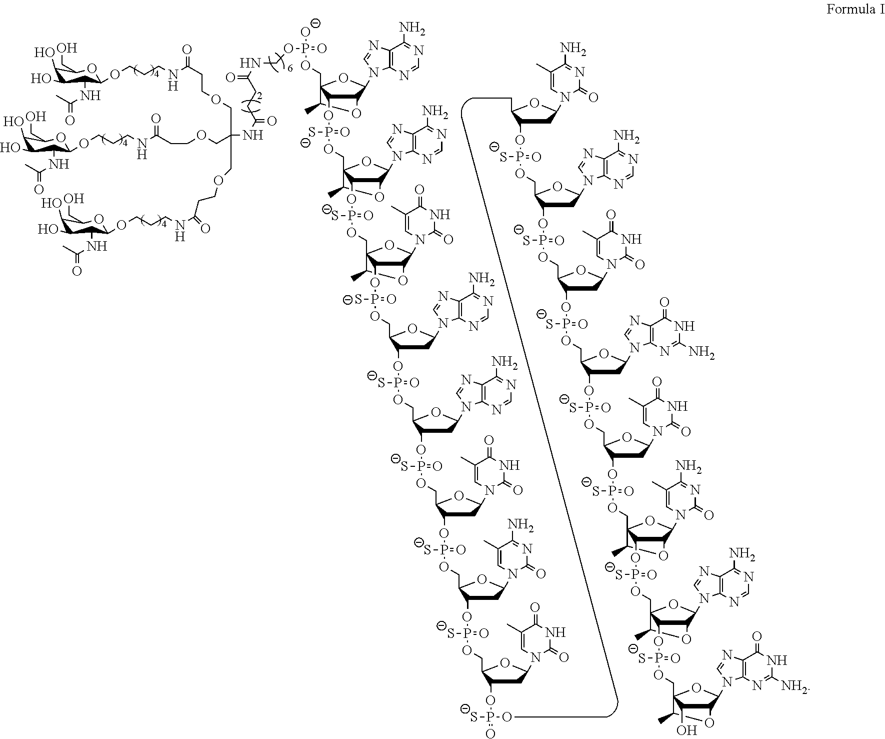

24. A method of reducing a low-density lipoprotein cholesterol (LDL-C) level in a subject, comprising administering to the subject a unit dosage comprising from about 10 mg to about 120 mg of a compound or pharmaceutically acceptable salt thereof, wherein the compound comprises a modified oligonucleotide and a conjugate group, wherein the modified oligonucleotide consists of SEQ ID NO:1 and comprises:
a gap segment consisting of ten linked deoxynucleosides;
a 5′ wing segment consisting of three linked nucleosides; and
10 a 3′ wing segment consisting of three linked nucleosides,
wherein the gap segment is positioned between the 5′ wing segment and the 3′ wing segment; wherein each nucleoside of each wing segment comprises a 2′-O-ethyl (cEt) sugar; wherein each internucleoside linkage is a phosphorothioate linkage; and wherein each cytosine is a 5-methylcytosine.

25. The method of claim 24, wherein the conjugate group comprises 5′-Trishexylamino-(THA)-C₆GalNAC3 linked to the 5′ end of the modified oligonucleotide.

26. The method of any of claims 23 to 25, wherein the level of LDL-C in the subject is reduced about 10% to about 90% after administration of the unit dosage as compared to a level of LDL-C in the subject before administration of the unit dosage.

27. The method of any of claims 23 to 26, wherein the level of LDL-C in the subject is reduced at least about 10%, about 20%, about 30%, about 40%, about 50%, about 60% or about 70% and up to about 80%, or about 90% after administration of the unit dosage as compared to a level of LDL-C in the subject before administration of the unit dosage.

28. The method of any of claims 23 to 27, wherein the level of LDL-C in the subject is reduced about 20% to about 80%, about 30% to about 70%, about 40% to about 70%, or about 50% to about 70% after administration of the unit dosage as compared to a level of LDL-C in the subject before administration of the unit dosage.

29. The method of any of claims 23 to 28, wherein the level of LDL-C in the subject is reduced for at least about 2 weeks after administering the unit dosage as compared to a level of LDL-C in the subject before administration of the unit dosage.

30. The method of claim 29, wherein the level of LDL-C in the subject is reduced for at least about 3 weeks, about 4 weeks, about 5 weeks or about 6 weeks after administering the unit dosage as compared to a level of LDL-C in the subject before administration of the unit dosage.

31. The method of any of claims 23 to 30, wherein a plasma level of LDL-C of the subject is reduced.

32. The method of any of claims 23 to 31, wherein the subject has a serum low-density lipoprotein cholesterol (LDL-C) level of at least about 100 mg/dL, at least about 110 mg/dL, at least about 120 mg/dL, at least about 130 mg/dL, at least about 140 mg/dL, at least about 150 mg/dL, at least about 160 mg/dL, at least about 170 mg/dL, at least about 180 mg/dL, at least about 190 mg/dL, at least about 200 mg/dL, at least about 300 mg/dL or at least about 400 mg/dL prior to administration of the unit dosage.

33. A method of treating a disease associated with PCSK9 in a subject, comprising administering to the subject from about 10 to about 120 mg of a compound of Formula I (SEQ ID NO:1), or pharmaceutically acceptable salt thereof:

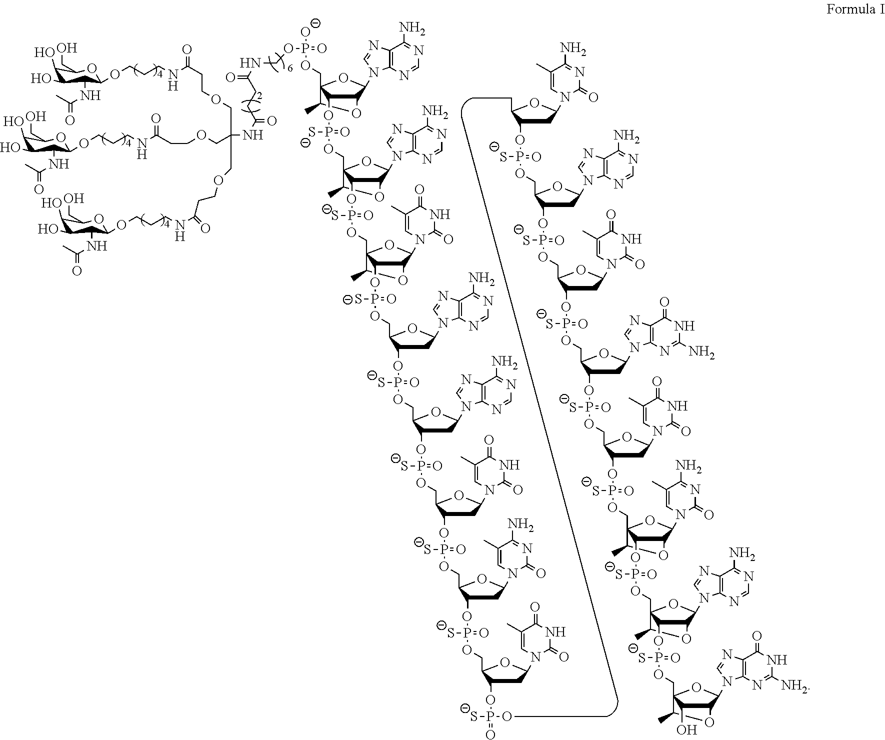

34. A method of treating a disease associated with PCSK9 in a subject, comprising administering to the subject a unit dosage comprising from about 10 mg to about 120 mg of a compound or pharmaceutically acceptable salt thereof, wherein the compound comprises a modified oligonucleotide and a conjugate group, wherein the modified oligonucleotide consists of SEQ ID NO:1 and comprises:
a gap segment consisting of ten linked deoxynucleosides;
a 5′ wing segment consisting of three linked nucleosides; and
a 3′ wing segment consisting of three linked nucleosides,
wherein the gap segment is positioned between the 5′ wing segment and the 3′ wing segment; wherein each nucleoside of each wing segment comprises a 2′-O-ethyl (cEt) sugar; wherein each internucleoside linkage is a phosphorothioate linkage; and wherein each cytosine is a 5-methylcytosine.

35. The method of claim 34, wherein the conjugate group comprises 5′-Trishexylamino-(THA)-C₆GalNAC3 linked to the 5′ end of the modified oligonucleotide.

36. The method of any of claims 33 to 35, wherein the disease associated with PCSK9 is selected from: elevated low-density lipoprotein cholesterol (LDL-C) levels, hyperlipidemia, dyslipidemia, atherosclerosis, cardiovascular disease, hypercholesterolemia, and combinations thereof.

37. The method of claim 36, wherein the disease associated with PCSK9 is dyslipidemia or hypercholesterolemia.

38. The method of any of claims 33 to 37, wherein a level of PCSK9 in the subject is reduced.

39. The method of claim 39, wherein the level of PCSK9 in the subject is reduced about 10% to about 95% after administration of the unit dosage as compared to a level of PCSK9 in the subject before administration of the unit dosage.

40. The method of any of claims 33 to 39, wherein the level of PCSK9 in the subject is reduced at least about 10%, about 20%, about 30%, about 40%, about 50%, about 60%, or about 70%, and up to about 80%, or about 90% after administration of the unit dosage as compared to a level of LDL-C in the subject before administration of the unit dosage.

41. The method of claim 38 or 40, wherein the level of PCSK9 in the subject is reduced about 10% to about 90%, about 20% to about 80%, about 30% to about 70%, about 40% to about 70%, or about 50% to about 70% after administration of the unit dosage as compared to a level of PCSK9 in the subject before administration of the unit dosage.

42. The method of any of claims 33 to 41, wherein the level of PCSK9 in the subject is reduced for at least about 2 weeks after administering the compound as compared to a level of PCSK9 in the subject before administration of the compound.

43. The method of claim 42, wherein the level of PCSK9 in the subject is reduced for at least about 3 weeks, at least about 4 weeks, at least about 5 weeks or at least about 6 weeks after administration of the unit dosage as compared to a level of PCSK9 in the subject before administration of the unit dosage.

44. The method of any of claims 33 to 43, wherein a plasma level of PCSK9 in the subject is reduced.

45. The method of any of claims 33 to 44, wherein PCSK9 expression is reduced.

46. The method of any of claims 23 to 45, wherein the unit dosage is administered to the subject about every 2 weeks to about every 6 weeks.

47. The method of claim 46, wherein the unit dosage is administered to the subject about every 2 weeks, about every 3 weeks, about every 4 weeks, about every 5 weeks or about every 6 weeks, for at least about 6 weeks, at least about 12 weeks, at least about 26 weeks, or at least about 52 weeks.

48. The method of any of claims 23 to 46, wherein the unit dosage is administered to the subject once a month for at least about 3 months.

49. The method of claim 48, wherein the unit dosage is administered to the subject once a month for at least about 6 months, at least about 9 months, at least about 12 months, at least about 15 months or at least about 24 months.

50. The method of any of claims 23 to 45, wherein the unit dosage is administered to the subject about every 21 days, about every 28 days, or about every 30 days for at least about 90 days, at least about 120 days, at least about 180 days, or at least about 365 days.

51. The method of any of claims 23 to 50, wherein the unit dosage is administered by parenteral administration.

52. The method of claim 51, wherein parenteral administration comprises subcutaneous, intravenous, or intramuscular administration.

53. The method of claim 52, wherein parenteral administration comprises subcutaneous administration.

54. The method of any of claims 23 to 53, wherein the subject is a human.

55. A dosing regimen for reducing a low-density lipoprotein cholesterol (LDL-C) level in a subject, the dosing regimen comprising:
(a) administering to the subject a loading dose comprising a unit dosage according to any of claims 1 to 15, wherein the LDL-C level in the subject is reduced about 10% to about 90% after administering the loading dose to provide a reduced LDL-C level in the subject; and
(b) administering one or more maintenance doses comprising a unit dosage according to any of claims 1 to 15.

56. The dosing regimen of claim 55, wherein the loading dose comprises a greater amount of a compound of Formula I (SEQ ID NO: 1) or a pharmaceutically acceptable salt thereof than the maintenance dose.

57. The dosing regimen of claim 55, wherein the loading dose comprises a same amount of a compound of Formula I (SEQ ID NO: 1) or a pharmaceutically acceptable salt thereof as the maintenance dose.

58. The dosing regimen of any of claims 55 to 57, wherein the maintenance dose is administered to the subject about every 2 weeks to about every 6 weeks.

59. The dosing regimen of claim 46, wherein the maintenance dose is administered to the subject about every 2 weeks, about every 3 weeks, about every 4 weeks, about every 5 weeks or about every 6 weeks, for at least about 6 weeks, at least about 12 weeks, at least about 26 weeks, or at least about 52 weeks.

60. The dosing regimen of any of claims 55 to 57, wherein the maintenance dose is administered to the subject once a month for at least about 3 months.

61. The dosing regimen of claim 60, wherein the maintenance dose is administered to the subject once a month for at least about at least about 6 months, at least about 9 months, at least about 12 months or at least about 15 months.

62. The dosing regimen of any of claims 55 to 57, wherein the maintenance dose is administered to the subject about every 21 days, about every 28 days, or about every 30 days for at least about 90 days, at least about 120 days, at least about 180 days, or at least about 365 days.

63. The dosing regimen of any of claims 55 to 62, wherein the loading dose, maintenance dose, or both are administered by parenteral administration.

64. The dosing regimen of claim 63, wherein parenteral administration comprises subcutaneous, intravenous, or intramuscular administration.

65. The dosing regimen of claim 63, wherein parenteral administration comprises subcutaneous administration.

66. The dosing regimen of any of claims 55 to 65, wherein the level of LDL-C in the subject is reduced at least about 20%, about 30%, about 40%, about 50%, about 60%, or about 70% and up to about 80%, or about 90% after administering the loading dose.

67. The dosing regimen of any of claims 55 to 66, wherein the level of LDL-C in the subject is reduced about 20% to about 80%, about 30% to about 70%, about 40% to about 70% or about 50% to about 70% after administering the loading dose.

68. The dosing regimen of any of claims 55 to 67, wherein the reduced LDL-C level in the subject increases less than about 10% for at least about 2 weeks after administering the loading dose.

69. The dosing regimen of any of claims 55 to 7, wherein the reduced LDL-C level in the subject increases less than about 10%, less than about 5% or less than about 2% for at least about 2 weeks, at least about 3 weeks, at least about 4 weeks, at least about 5 weeks, and up to about 6 weeks after administering the loading dose.

70. The dosing regimen of any of claims 55 to 69, wherein the reduced LDL-C level in the subject increases less than about 10% for at least about 2 weeks after administering the maintenance dose.

71. The dosing regimen of any of claims 55 to 70, wherein the reduced LDL-C level in the subject increases less than about 10%, less than about 5% or less than about 2% for at least about 2 weeks, at least about 3 weeks, at least about 4 weeks, at least about 5 weeks, and up to about 6 weeks after administering the maintenance dose.

72. The dosing regimen of any of claims 55 to 71, wherein a plasma level of LDL-C in the subject is reduced.

73. The dosing regimen of any of claims 55 to 72, wherein the subject has a serum low-density lipoprotein cholesterol (LDL-C) level of at least about 100 mg/dL, at least about 110 mg/dL, at least about 120 mg/dL, at least about 130 mg/dL, at least about 140 mg/dL, at least about 150 mg/dL, at least about 160 mg/dL, at least about 170 mg/dL, at least about 180 mg/dL, at least about 190 mg/dL, at least about 200 mg/dL, at least about 300 mg/dL or at least about 400 mg/dL prior to administration of the loading dose.

74. The dosing regimen of any of claims 55 to 74, wherein the subject is a human.

75. A dosing regimen for reducing a level of PCSK9 in a subject, the dosing regimen comprising:
(a) administering to the subject a loading dose comprising a unit dosage according to any of claims 1 to 15, wherein the level of PCSK9 in the subject is reduced about 10% to about 95% after administering the loading dose to provide a reduced PCSK9 level in the subject; and
(b) administering one or more maintenance doses comprising a unit dosage according to any of claims 1 to 15.

76. The dosing regimen of claim 75, wherein the loading dose comprises a greater amount of a compound of Formula I (SEQ ID NO: 1) or a pharmaceutically acceptable salt thereof than the maintenance dose.

77. The dosing regimen of claim 75, wherein the loading dose comprises a same amount of a compound of Formula I (SEQ ID NO: 1) or a pharmaceutically acceptable salt thereof as the maintenance dose.

78. The dosing regimen of any of claims 75 to 77, wherein the maintenance dose is administered to the subject about every 2 weeks to about every 6 weeks.

79. The dosing regimen of claim 78, wherein the maintenance dose is administered to the subject from about every 2 weeks, about every 3 weeks, about every 4 weeks, about every 5 weeks and up to about every 6 weeks, for at least about 6 weeks, at least about 12 weeks, at least about 26 weeks, or at least about 52 weeks.

80. The dosing regimen of any of claims 75 to 77, wherein the maintenance dose is administered to the subject about once a month for at least about 3 months.

81. The dosing regimen of claim 80, wherein the maintenance dose is administered to the subject about once a month for at least about 6 months, at least about 9 months, at least about 12 months, at least about 15 months, or at least about 24 months.

82. The dosing regimen of any of claims 75 to 81, wherein the maintenance dose is administered to the subject about every 21 days, about every 28 days, or about every 30 days for at least about 90 days, at least about 120 days, at least about 180 days, or at least about 365 days.

83. The dosing regimen of any of claims 75 to 82, wherein the loading dose, maintenance dose or both are administered by parenteral administration.

84. The dosing regimen of claim 83, wherein parenteral administration comprises subcutaneous, intravenous, or intramuscular administration.

85. The dosing regimen of claim 84, wherein parenteral administration comprises subcutaneous administration.

86. The dosing regimen of any of claims 75 to 85, wherein the level of PCSK9 in the subject is reduced at least about 10%, about 20%, about 30%, about 40%, about 50%, about 60%, or about 70%, and up to about 80%, or about 90% after administering the loading dose.

87. The dosing regimen of any of claims 75 to 86, wherein the level of PCSK9 in the subject is reduced about 10% to about 90%, about 20% to about 80%, about 30% to about 70%, about 40% to about 70% or about 50% to about 70% after administering the loading dose.

88. The dosing regimen of any of claims 75 to 87, wherein the reduced PCSK9 level in the subject increases less than about 10%, less than about 5% or less than about 2% for at least about 2 weeks, at least about 3 weeks, at least about 4 weeks, at least about 5 weeks, or at least about 6 weeks after administering the loading dose.

89. The dosing regimen of any of claims 75 to 88, wherein the reduced PCSK9 level in the subject increases less than about 10%, about 5% or about 2% for at least about 2 weeks, about 3 weeks, about 4 weeks, about 5 weeks, or 6 about weeks after administration of the maintenance dose.

90. The dosing regimen of any of claims 75 to 89, wherein a plasma level of PCSK9 in the subject is reduced.

91. The dosing regimen of any of claims 75 to 90, wherein PCSK9 expression is reduced.

92. The dosing regimen of any of claims 75 to 91, comprising administering 2 or more maintenance doses.

93. The dosing regimen of any one of claims 75 to 92, comprising administering 3 or more, 4 or more, 5 or more, 6 or more, 9 or more, 12 or more, or 15 or more maintenance doses.

94. The dosing regimen of claim 92 or 93, wherein one maintenance dose is administered about every 3 weeks to about every 6 weeks.

95. The dosing regimen of claim 94, wherein the maintenance dose is administered for at least about 3 months, about 6 months, or about 1 year.

96. The dosing regimen of claim 95, wherein the reduced LDL-C level in the subject increases less than about 10%, about 5% or about 2% after about 6 weeks, after about 9 weeks, or after about 12 weeks after administration of a loading dose.

97. The dosing regimen of claim 95, wherein the reduced PCSK9 level in the subject increases less than about 10%, about 5% or about 2% after about 6 weeks, after about 9 weeks, or after about 12 weeks after administration of a maintenance dose.

98. The dosing regimen of any of claims 75 to 97, wherein the subject is a human.

99. A single dose container comprising from about 10 mg to about 120 mg of a compound of formula I (SEQ ID NO:1) or a pharmaceutically acceptable salt thereof:

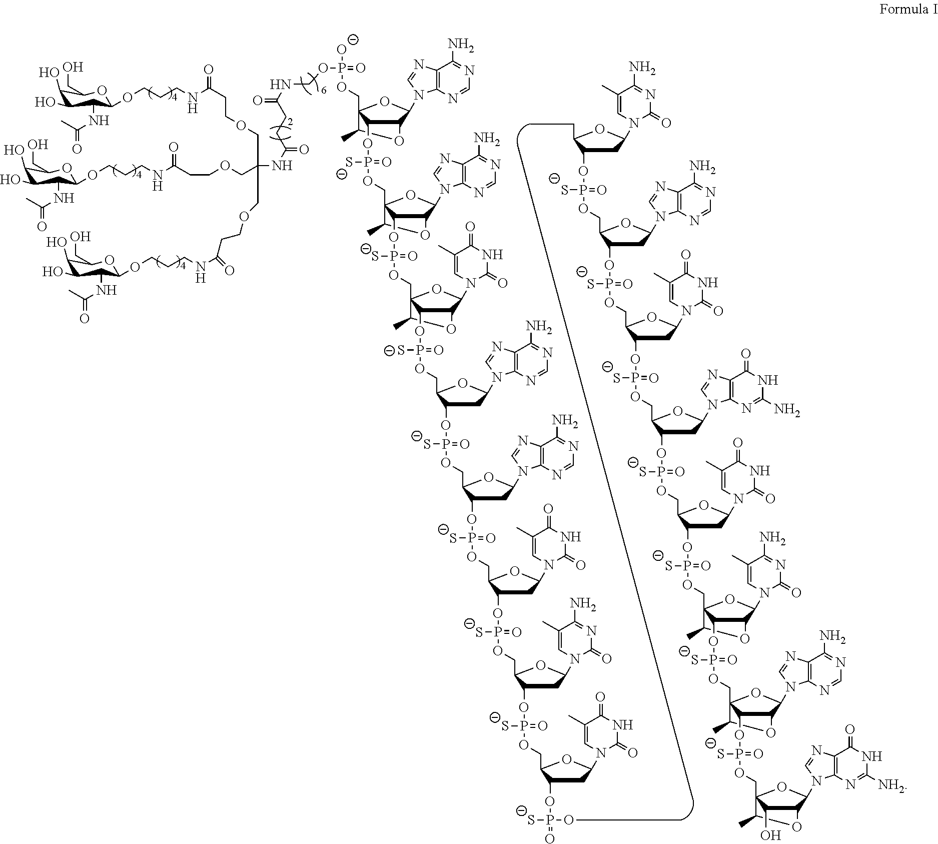

100. A single dose container comprising from about 10 mg to about 120 mg of a compound or pharmaceutically acceptable salt thereof, wherein the compound comprises a modified oligonucleotide and a conjugate group, wherein the modified oligonucleotide consists of SEQ ID NO:1 and comprises:
5 a gap segment consisting of ten linked deoxynucleosides;
a 5′ wing segment consisting of three linked nucleosides; and
a 3′ wing segment consisting of three linked nucleosides,
wherein the gap segment is positioned between the 5′ wing segment and the 3′ wing segment; wherein each nucleoside of each wing segment comprises a 2′-O-ethyl (cEt) sugar; wherein each internucleoside linkage is a phosphorothioate linkage; and wherein each cytosine is a 5-methylcytosine.

101. The single dose container of claim 100, wherein the conjugate group comprises 5′-Trishexylamino-(THA)-C₆GalNAC3 linked to the 5′ end of the modified oligonucleotide.

102. The single dose container of any of claims 99 to 101, comprising a single-dose prefilled syringe.
